(19) United States
(12) Patent Application Publication
(10) Pub. No.: US 2012/0039884 A1
(43) Pub. Date: Feb. 16, 2012

(54) COMPOSITIONS AND METHODS FOR TREATING CARDIOVASCULAR DISEASE

(75) Inventors: Richard L. Watson, McPherson, KS (US); Anthony B. Wood, Dallas, TX (US); Gregory J. Archambeau, Puyallup, WA (US)

(73) Assignee: Revalesio Corporation, Tacoma, WA (US)

(21) Appl. No.: 13/209,154

(22) Filed: Aug. 12, 2011

Related U.S. Application Data
(60) Provisional application No. 61/373,494, filed on Aug. 13, 2010.
(60) Provisional application No. 61/485,071, filed on May 11, 2011.

(51) Int. Cl. A61K 33/00; A61P 35/00; A61P 29/00; A61P 25/00; A61K 33/24; A61P 25/16; A61P 37/02; A61P 9/10; A61K 39/395; A61P 11/00; A61P 25/18

(52) U.S. Cl. 424/134.1; 424/600; 424/158.1; 424/649

(57) ABSTRACT

Provided are methods for treating cardiovascular diseases and related conditions and symptoms (e.g., cardiac arrhythmia, vascular disease, myocardial infarction, congestive heart failure, myocarditis, atherosclerosis, and restenosis), comprising administering to a subject in need thereof a therapeutically effective amount of an electrokinetically altered aqueous fluid as described herein. In particular aspects, the electrokinetically altered aqueous fluids comprise an ionic aqueous solution of charge-stabilized oxygen-containing nanostructures predominantly having an average diameter of less than about 100 nanometers and sufficient to provide modulation of at least one of cellular membrane potential and cellular membrane conductivity. Provided are routes of administration or formulations for the electrokinetically-altered fluids (e.g., electrokinetically-altered gas-enriched fluids and solutions) and therapeutic compositions, along with use of the electrokinetically altered aqueous fluids in surgical contexts, including but not limited to cardiovascular related surgeries. Additionally provided are methods for measuring biological activity of electrokinetically altered fluids.

### CROSS-REFERENCE TO RELATED APPLICATIONS

This application claims the benefit of priority to U.S. Provisional Patent Application Ser. No. 61/373,494 filed Aug. 13, 2010, and 61/485,071 filed May 11, 2011, both incorporated by reference herein in their entirety.

### FIELD OF THE INVENTION

Particular aspects relate generally to methods for treating cardiovascular diseases and related conditions and symptoms thereof (e.g., at least one of cardiac arrhythmia, vascular disease, myocardial infarction, congestive heart failure, myocarditis, atherosclerosis, and restenosis), comprising administering to a subject in need thereof a therapeutically effective amount of an electrokinetically altered aqueous fluid as described herein. Certain aspects relate to use of electrokinetically altered aqueous fluids comprising an ionic aqueous solution of charge-stabilized oxygen-containing nanostructures substantially having an average diameter of less than about 100 nanometers stably configured in the ionic aqueous fluid in an amount sufficient to provide, upon contact of a living cell by the fluid, modulation of at least one of cellular membrane potential and cellular membrane conductivity. Further aspects relate to methods for measuring biological activity of electrokinetically altered fluids.

### BACKGROUND OF THE INVENTION

Cardiovascular diseases are a large class of diseases that involve the heart or blood vessels (arteries and veins). Cardiovascular diseases include, but are not limited to cardiac arrhythmia, vascular disease, myocardial infarction, congestive heart failure, myocarditis, atherosclerosis, and restenosis. These conditions have similar causes, mechanisms, and treatments. Most cardiovascular diseases share common risk factors, including inflammation, high cholestrol, and obesity.

Additionally, there are many cardiovascular surgical scenarios in which saline solutions are used and wherein it would be desirable to reduce or eliminate deleterious effects attendant to the surgical scenarious (e.g., caridoplumonary bypass (CPB) prime (bypass pump priming solution) where generalize inflammatory response causes deleterious effects of CPB; vein preservation solution (typically papaverine and saline) cadioplegia(e.g., glutamate and/or aspartate-containing cardiplegia, that may also contain potassium) to flush down coronaries after grafts are completed that benefit from the use of saline solutions).

### SUMMARY OF THE INVENTION

Provided are methods for treating cardiovascular diseases and related conditions and symptoms thereof (e.g., at least one condition or disease selected from the group consisting of cardiac arrhythmia, vascular disease, myocardial infarction, congestive heart failure, myocarditis, atherosclerosis, and restenosis), comprising administering to a subject in need thereof a therapeutically effective amount of an electrokinetically altered aqueous fluid as described herein. In particular aspects, the electrokinetically altered aqueous fluids comprise an ionic aqueous solution of charge-stabilized oxygen-containing nanostructures substantially (e.g., predominantly) having an average diameter of less than about 100 nanometers and sufficient to provide modulation of at least one of cellular membrane potential and cellular membrane conductivity. Additionally provided are routes of administration or formulations for the electrokinetically-altered fluids (e.g., electrokinetically-altered gas-enriched fluids and solutions) and therapeutic compositions, along with use of the electrokinetically altered aqueous fluids in surgical contexts, including but not limited to cardiovascular related surgeries.

Particular aspects provide methods for treating a cardiovascular disease or condition, comprising administering to a subject, or portion thereof, in need thereof a therapeutically effective amount of an electrokinetically altered aqueous fluid comprising an ionic aqueous solution of charge-stabilized oxygen-containing nanostructures substantially (e.g., predominantly) having an average diameter of less than about 100 nanometers and stably configured in the ionic aqueous fluid in an amount sufficient to provide for treating a cardiovascular disease or condition or at least one symptom thereof In certain aspects, the charge-stabilized oxygen-containing nanostructures are stably configured in the ionic aqueous fluid in an amount sufficient to provide, upon contact of a living cell by the fluid, modulation of at least one of cellular membrane potential and cellular membrane conductivity.

In particular embodiments, the charge-stabilized oxygen-containing nanostructures are the major charge-stabilized gas-containing nanostructure species in the fluid. In certain aspects, the percentage of dissolved oxygen molecules present in the fluid as the charge-stabilized oxygen-containing nanostructures is a percentage selected from the group consisting of greater than: 0.01%, 0.1%, 1%, 5%; 10%; 15%; 20%; 25%; 30%; 35%; 40%; 45%; 50%; 55%, 60%; 65%; 70%; 75%; 80%; 85%; 90%; and 95%. In particular aspects, the total dissolved oxygen is substantially present in the charge-stabilized oxygen-containing nanostructures. In ceratin embodiments, the charge-stabilized oxygen-containing nanostructures substantially have an average diameter of less than a size selected from the group consisting of: 90 nm; 80 nm; 70 nm; 60 nm; 50 nm; 40 nm; 30 nm; 20 nm; 10 nm; and less than 5 nm.

In preferred embodiments, the ionic aqueous solution comprises a saline solution. In certain embodiments, the fluid is superoxygenated.

In particular aspects, the fluid comprises a form of solvated electrons.

In certain aspects, alteration of the electrokinetically altered aqueous fluid comprises exposure of the fluid to hydrodynamically-induced, localized electrokinetic effects. In particular embodiments, exposure to the localized electrokinetic effects comprises exposure to at least one of voltage pulses and current pulses. In particular embodiments, exposure of the fluid to hydrodynamically-induced, localized electrokinetic effects, comprises exposure of the fluid to electrokinetic effect-inducing structural features of a device used to generate the fluid.

In certain embodiments, the cardiovascular disease or condition comprises at least one condition or disease selected from the group consisting of cardiac arrhythmia, vascular disease, myocardial infarction, congestive heart failure, myocarditis, atherosclerosis, and restenosis. In particular embodiments, the cardiovascular condition or disease comprises at least one of myocardial infarction, congestive heart failure, myocarditis, and atherosclerosis. In preferred aspects, the cardiovascular condition or disease comprises at least one of myocardial infarction and atherosclerosis.

In particular aspects, the at least one symptom of cardiovascular disease is related to at least one condition selected from the group consisting of: cardiac arrhythmia, vascular disease, myocardial infarction, congestive heart failure, myocarditis, atherosclerosis, and restenosis.

In certain aspects, the electrokinetically altered aqueous fluid modulates localized or cellular levels of nitric oxide.

In particular aspects, the electrokinetically altered aqueous fluid promotes a localized decrease at the site of administration of at least one cytokine selected from the group consisting of: IL-1beta, IL-8, TNF-alpha, and TNF-beta.

Particular aspects further comprise a synergistic or non-synergistic inhibition or reduction in inflammation by simultaneously or adjunctively treating the subject with another anti-inflammatory agent. In certain embodiments, said other anti-inflammatory agent comprises a steroid or glucocorticoid steroid (e.g., comprising Budesonide or an active derivative thereof).

Particular aspects further comprise combination therapy, wherein at least one additional therapeutic agent is administered to the patient. In certain embodiments, the at least one additional therapeutic agent is selected from the group consisting of: quinidine, procainamide, disopyramide, lidocaine, phenytoin, mexiletine, flecainide, propafenone, moricizine, propranolol, esmolol, timolol, metoprolol, atenolol, bisoprolo, amiodarone, sotalol, ibutilide, dofetilide, dronedarone, E-4031, verapamil, diltiazem, adenosine, digoxin, magnesium sulfate, warfarin, heparins, anti-platelet drugs (e.g., aspirin and clopidogrel), beta blockers (e.g., metoprolol and carvedilol), angiotensin-converting enzyme (ACE) inhibitors (e.g., captopril, zofenopril, enalapril, ramipril, quinapril, perindopril, lisinopril, benazepril, fosinopril, casokinins and lactokinins), statins (e.g., atorvastatin, cerivastatin, fluvastatin, lovastatin, pitavastatin, mevastatin, pravastatin, rosuvastatin, and simvastatin), aldosterone antagonist agents (e.g., eplerenone and spironolactone), digitalis, diuretics, digoxin, inotropes (e.g., Milrinone), vasodilators and omega-3 fatty acids and combinations thereof.

In certain embodiments, the at least one additional therapeutic agent comprises a TSLP and/or TSLPR antagonist. In particular aspects, the TSLP and/or TSLPR antagonist is selected from the group consisting of neutralizing antibodies specific for TSLP and the TSLP receptor, soluble TSLP receptor molecules, and TSLP receptor fusion proteins, including TSLPR-immunoglobulin Fc molecules or polypeptides that encode components of more than one receptor chain.

In particular aspects, modulation of at least one of cellular membrane potential and cellular membrane conductivity comprises modulating at least one of cellular membrane structure or function comprising modulation of a conformation, ligand binding activity, or a catalytic activity of a membrane associated protein. In certain embodiments, the membrane associated protein comprises at least one selected from the group consisting of receptors, transmembrane receptors, ion channel proteins, intracellular attachment proteins, cellular adhesion proteins, integrins, etc. In particular aspects, the transmembrane receptor comprises a G-Protein Coupled Receptor (GPCR). In certain embodiments, the G-Protein Coupled Receptor (GPCR) interacts with a G protein α subunit. In particular aspects, the G protein α subunit comprises at least one selected from the group consisting of Gαₛ, Gαᵢ, Gα_{q}, and Gα₁₂. In certain embodiments, the at least one G protein α subunit is Gα_{q}.

In particular aspects, modulating cellular membrane conductivity, comprises modulating whole-cell conductance. In certain embodiments, modulating whole-cell conductance, comprises modulating at least one voltage-dependent contribution of the whole-cell conductance.

In certain aspects, modulation of at least one of cellular membrane potential and cellular membrane conductivity comprises modulating intracellular signal transduction comprising modulation of a calcium dependant cellular messaging pathway or system. In certain aspects, modulation of at least one of cellular membrane potential and cellular membrane conductivity comprises modulating intracellular signal transduction comprising modulation of phospholipase C activity. In certain aspects, modulation of at least one of cellular membrane potential and cellular membrane conductivity comprises modulating intracellular signal transduction comprising modulation of adenylate cyclase (AC) activity. In certain aspects, modulation of at least one of cellular membrane potential and cellular membrane conductivity comprises modulating intracellular signal transduction comprising modulation of intracellular signal transduction associated with at least one condition or symptom selected from the group consisting of: chronic inflammation in the cardiovascular system, and acute inflammation in the cardiovascular system.

In particular aspects, the methods comprise administration to a cell network or layer, and further comprising modulation of an intercellular junction therein. In certain embodiments, the intracellular junction comprises at least one selected from the group consisting of tight junctions, gap junctions, zona adherins and desmasomes. In particular embodiments, the cell network or layers comprises at least one selected from the group consisting of endothelial cell and endothelial-astrocyte tight junctions in CNS vessels, blood-cerebrospinal fluid tight junctions or barrier, pulmonary epithelium-type junctions, bronchial epithelium-type junctions, and intestinal epithelium-type junctions.

In particular aspects, the electrokinetically altered aqueous fluid is oxygenated, and wherein the oxygen in the fluid is present in an amount of at least 8 ppm, at least 15, ppm, at least 25 ppm, at least 30 ppm, at least 40 ppm, at least 50 ppm, or at least 60 ppm oxygen at atmospheric pressure. In certain aspects, the amount of oxygen present in charge-stabilized oxygen-containing nanostructures of the electrokinetically-altered fluid is at least 8 ppm, at least 15, ppm, at least 20 ppm, at least 25 ppm, at least 30 ppm, at least 40 ppm, at least 50 ppm, or at least 60 ppm oxygen at atmospheric pressure.

In certain aspects, the electrokinetically altered aqueous fluid comprises at least one of a form of solvated electrons, and electrokinetically modified or charged oxygen species. In particular embodiments, the form of solvated electrons or electrokinetically modified or charged oxygen species are present in an amount of at least 0.01 ppm, at least 0.1 ppm, at least 0.5 ppm, at least 1 ppm, at least 3 ppm, at least 5 ppm, at least 7 ppm, at least 10 ppm, at least 15 ppm, or at least 20 ppm. In particular embodiments, the electrokinetically altered oxygenated aqueous fluid comprises solvated electrons stabilized, at least in part, by molecular oxygen.

In particular aspects, the ability to alter cellular membrane structure or function sufficient to provide for modulation of intracellular signal transduction persists for at least two, at least three, at least four, at least five, at least 6, at least 12 months, or longer periods, in a closed gas-tight container.

In certain aspects, the membrane associated protein comprises CCR3.

In particular aspects, treating comprises modulation of intracellular NF-κB expression and/or activity.

In preferred aspects, the subject is a mammal or human.

Particular aspects provide methods for performing a surgery, comprising performing a surgery on a subject in need thereof, wherein a reagent fluid is used in at least one aspect of the surgery, and wherein the reagent fluid comprises a surgically effective amount of an electrokinetically altered aqueous fluid comprising an ionic aqueous solution of charge-stabilized oxygen-containing nanostructures substantially having an average diameter of less than about 100 nanometers.

In particular aspects, the surgery comprises at least one selected from the group consisting of: surgery related to cardiac arrhythmia; surgery related to vascular disease; surgery related to myocardial infarction; surgery related to congestive heart failure; surgery related to myocarditis; surgery related to atherosclerosis, and restenosis; surgery comprising use of caridoplumonary bypass (CPB); surgery comprising use of vessel (e.g., vein, artery) preservation solution; and surgery comprising use of cadioplegia.

Additional aspects provide methods for facile high-throughput measurement of biological activity of electronkinetically-altered fluids (e.g., RNS60), comprising: contacting a cell with an electronkinetically-altered fluid as defined herein; performing, using a suitable assay, an ion-channel measurement; and determining, based on the ion-channel measurement relative to that of cells contacted with control fluid, a biological acitivty level or value of the electronkinetically-altered fluid. In certain embodiments, the ion-channel measurement is at least one selected from the group consisting of potentiation, inhibition, alteration of gating kinetics, voltage sensitivity, and modulation of agonist-evoked acitivity. In particular aspects, the ion channel is at least one of 5HT3A and TRPV1. In certain embodiments, the methods comprise measurement of at least one of serotonin-evoked 5HT3A and capsaicin evoked TRPV1.

Yet further aspects provide facile high-throughput methods for measurement of biological activity of electronkinetically-altered fluids (e.g., RNS60), comprising: contacting a cell with an electronkinetically-altered fluid as defined herein; performing at least one of Raman spectroscopy and fluorescence porlarizatoin anisotropy mearsurement; and determining, based on the at least one measurement relative to that of cells contacted with control fluid, a biological acitivty level or value of the electronkinetically-altered fluid. In certain embodiments, the methods comprise measurement of at least one of Raman backscatter and fluorescence porlarizatoin anisotropy.

### BRIEF DESCRIPTION OF THE DRAWINGS

FIG. 1 illustrates the cytokine profile of a mitogenic assay in the presence of a gas-enriched fluid and deionized control fluid.

FIGS. 2-11 show the results of whole blood sample evaluations of cytokines.

FIGS. 12-21 show the corresponding cytokine results of bronchoalveolar lavage fluid (BAL) sample evaluations.

FIGS. 22-29 show data showing the ability of particular embodiments disclosed herein to affect regulatory T cells. The study involved irradiating antigen presenting cells, and introducing antigen and T cells.

FIGS. 30-34 show data obtained from human foreskin keratinocytes exposed to RDC1676-01 (sterile saline processed through the instant proprietary device with additional oxygen added; gas-enriched electrokinetically generated fluid (Rev)).

FIGS. 35-38 show results of budesonide in combination with the inventive electrokinetically generated fluids experiments performed to assess the airway anti-inflammatory properties of the inventive electrokinetically generated fluids in a Brown Norway rat ovalbumin sensitization model. The inventive electrokinetically generated fluids decreased eosinophil count, showed strong synergy with Budesonide in decreasing eosinophil count, decreased blood levels of Eotaxin, significantly enhanced the blood levels of two major key anti-inflammatory cytokines, IL10 and Interferon gamma at 6 hours after challenge as a result of treatment with the inventive electrokinetically generated fluid (e.g., RNS-60) alone or in combination with Budesonide, and decreased systemic levels of Rantes.

FIG. 39 shows the inventive electrokinetically generated fluid (e.g., RNS-60 and Solas) inhibited the DEP-induced cell surface bound MMP-9 levels in bronchial epithelial cells by approximately 80%, and 70%, respectively, whereas normal saline (NS) had only a marginal effect.

FIGS. 40A-B demonstrate the results of Fluorescence-Activated Cell Sorting (FACS) analysis wherein the levels of expression of the cell surface receptor, CD193 (CCR3), on white blood cells was compared using either normal saline or RNS-60. The X-axis represents the log fluorescence of the sample and the Y-axis represents the events of fluorescence that occur in the sample.

FIGS. 41A-C demonstrate the results of Fluorescence-Activated Cell Sorting (FACS) analysis wherein the levels of expression of cell surface receptors, CD154 (CD40L) (panel A); CD11B (panel B); and CD3 (panel C), on white blood cells was compared using either normal saline or RNS-60. The X-axis represents the log fluorescence of the sample and the Y-axis represents the events of fluorescence that occur in the sample.

FIGS. 42A-C) show the results from two gel shift experiments (panels A and B) and a luciferase activity (reporter gene) assay (panel C) that examined the effects of RNS60 on the activation of NFκB in MBP-primed T cells.

FIGS. 43A-D is a graphical representation of a time course of the blood level of troponin (panels A and B) and creatine phosphokinase (CPK) (panels C and D) upon induction of myocardial infarction.

FIGS. 44A-I show, according to particular aspects, an example of the necrosis tissue found in a control-treated male animal (#3033).

FIGS. 45A and B show, according to particular aspects, the effect of increased temperature (heart) on RNS60 (45B) relative to control PNS60 (45A), as measured by Raman backscatter, showing respective difference curves, and two oxygen peaks.

FIG. 46 shows, according to particular aspects, small but significant differences in fluorescence porlarizatoin anisotropy data between and among “RNS60” (“Lot A” and “Lot B”), “NS” (normal saline control), “RDW” (electrokinetically processed deionized water) and “DW” (deionized water).

FIG. 47 shows, according to particular aspects, that extracellularly perfused RNS60 (89%) potentiates serotonin-evoked 5HT3A (ion channel) activity (avg. inhibition of −101.8±24.2% (n=3), relative to control).

FIG. 48 shows, according to particular aspects, that RNS60 (84%) inhibits capsaicin evoked TRPV1 (ion channel) currents (avg. inhibition of −90.9±6.7% (n=3). Comparison is between Normal Saline, 100 nm Capsaicin, and 100 nm Capsaicin+87% RNS60.

FIG. 49 shows, according to particular aspects, that perfusion with RNS60 alters electrical spiking in cardiomyocytes; Vᵣₐₘₚ−100 mV→+40 mV; ΔVₛₚᵢₖₑ, 1.67±0.47 mV; Δtₛₚᵢₖₑ, 5.95 1.67 msec (n=6). Without being bound by mechanism, delay of spiking may be due to interaction with Nav1.5.

### DETAILED DESCRIPTION OF THE INVENTION

Provided are methods for treating cardiovascular diseases and related conditions and symptoms thereof (e.g., at least one condition or disease selected from the group consisting of cardiac arrhythmia, vascular disease, myocardial infarction, congestive heart failure, myocarditis, atherosclerosis, and restenosis), comprising administering to a subject in need thereof a therapeutically effective amount of an electrokinetically altered aqueous fluid as described herein. In particular aspects, the electrokinetically altered aqueous fluids comprise an ionic aqueous solution of charge-stabilized oxygen-containing nanostructures substantially having an average diameter of less than about 100 nanometers stably configured in the ionic aqueous fluid in an amount sufficient to provide, upon contact of a living cell by the fluid, modulation of at least one of cellular membrane potential and cellular membrane conductivity. Other embodiments include particular routes of administration or formulations for the electrokinetically-altered fluids (e.g., electrokinetically-altered gas-enriched fluids and solutions) and therapeutic compositions.

### Electrokinetically-Generated Fluids:

“Electrokinetically generated fluid,” as used herein, refers to Applicants' inventive electrokinetically-generated fluids generated, for purposes of the working Examples herein, by the exemplary Mixing Device described in detail herein (see also US200802190088 and WO2008/052143, both incorporated herein by reference in their entirety). The electrokinetic fluids, as demonstrated by the data disclosed and presented herein, represent novel and fundamentally distinct fluids relative to prior art non-electrokinetic fluids, including relative to prior art oxygenated non-electrokinetic fluids (e.g., pressure pot oxygenated fluids and the like). As disclosed in various aspects herein, the electrokinetically-generated fluids have unique and novel physical and biological properties including, but not limited to the following:

In particular aspects, the electrokinetically altered aqueous fluid comprise an ionic aqueous solution of charge-stabilized oxygen-containing nanostructures substantially having an average diameter of less than about 100 nanometers and stably configured in the ionic aqueous fluid in an amount sufficient to provide, upon contact of a living cell by the fluid, modulation of at least one of cellular membrane potential and cellular membrane conductivity.

In particular aspects, electrokinetically-generated fluids refers to fluids generated in the presence of hydrodynamically-induced, localized (e.g., non-uniform with respect to the overall fluid volume) electrokinetic effects (e.g., voltage/current pulses), such as device feature-localized effects as described herein. In particular aspects said hydrodynamically -induced, localized electrokinetic effects are in combination with surface-related double layer and/or streaming current effects as disclosed and discussed herein.

In particular aspects, the electrokinetically altered aqueous fluids are suitable to modulate ¹³C-NMR line-widths of reporter solutes (e.g., Trehelose) dissolved therein. NMR line-width effects are in indirect method of measuring, for example, solute ‘tumbling’ in a test fluid as described herein in particular working Examples.

In particular aspects, the electrokinetically altered aqueous fluids are characterized by at least one of: distinctive square wave voltametry peak differences at any one of −0.14V, −0.47V, −1.02V and −1.36V; polarographic peaks at −0.9 volts; and an absence of polarographic peaks at −0.19 and −0.3 volts, which are unique to the electrokinetically generated fluids as disclosed herein in particular working Examples.

In particular aspects, the electrokinetically altered aqueous fluids are suitable to alter cellular membrane conductivity (e.g., a voltage-dependent contribution of the whole-cell conductance as measure in patch clamp studies disclosed herein).

In particular aspects, the electrokinetically altered aqueous fluids are oxygenated, wherein the oxygen in the fluid is present in an amount of at least 15, ppm, at least 25 ppm, at least 30 ppm, at least 40 ppm, at least 50 ppm, or at least 60 ppm dissolved oxygen at atmospheric pressure. In particular aspects, the electrokinetically altered aqueous fluids have less than 15 ppm, less that 10 ppm of dissolved oxygen at atmospheric pressure, or approximately ambient oxygen levels.

In particular aspects, the electrokinetically altered aqueous fluids are oxygenated, wherein the oxygen in the fluid is present in an amount between approximately 8 ppm and approximately 15 ppm, and in this case is sometimes referred to herein as “Solas.”

In particular aspects, the electrokinetically altered aqueous fluid comprises at least one of solvated electrons (e.g., stabilized by molecular oxygen), and electrokinetically modified and/or charged oxygen species, and wherein in certain embodiments the solvated electrons and/or electrokinetically modified or charged oxygen species are present in an amount of at least 0.01 ppm, at least 0.1 ppm, at least 0.5 ppm, at least 1 ppm, at least 3 ppm, at least 5 ppm, at least 7 ppm, at least 10 ppm, at least 15 ppm, or at least 20 ppm.

In particular aspects, the electrokinetically altered aqueous fluids are characterized by differential (e.g., increased or decreased) permittivity relative to control, non-electrokinetically altered fluids. In preferred aspects, the electrokinetically altered aqueous fluids are characterized by differential, increased permittivity relative to control, non-electrokinetically altered fluids. Permittivity (c) (farads per meter) is a measure of the ability of a material to be polarized by an electric field and thereby reduce the total electric field inside the material. Thus, permittivity relates to a material's ability to transmit (or “permit”) an electric field. Capacitance (C) (farad; coulomb per volt), a closely related property, is a measure of the ability of a material to hold charge if a voltage is applied across it (e.g., best modeled by a dielectric layer sandwiched between two parallel conductive plates). If a voltage V is applied across a capacitor of capacitance C, then the charge Q that it can hold is directly proportional to the applied voltage V, with the capacitance C as the proportionality constant. Thus, Q=CV, or C=Q/V. The capacitance of a capacitor depends on the permittivity ε of the dielectric layer, as well as the area A of the capacitor and the separation distance d between the two conductive plates. Permittivity and capacitance are mathematically related as follows: C=ε (A/d). When the dielectric used is vacuum, then the capacitance Co=εo (A/d), where εo is the permittivity of vacuum (8.85×10⁻¹² F/m). The dielectric constant (k), or relative permittivity of a material is the ratio of its permittivity ε to the permittivity of vacuum εo, so k=ε/εo (the dielectric constant of vacuum is 1). A low-k dielectric is a dielectric that has a low permittivity, or low ability to polarize and hold charge. A high-k dielectric, on the other hand, has a high permittivity. Because high-k dielectrics are good at holding charge, they are the preferred dielectric for capacitors. High-k dielectrics are also used in memory cells that store digital data in the form of charge.

In particular aspects, the electrokinetically altered aqueous fluids are suitable to alter cellular membrane structure or function (e.g., altering of a conformation, ligand binding activity, or a catalytic activity of a membrane associated protein) sufficient to provide for modulation of intracellular signal transduction, wherein in particular aspects, the membrane associated protein comprises at least one selected from the group consisting of receptors, transmembrane receptors (e.g., G-Protein Coupled Receptor (GPCR), TSLP receptor, beta 2 adrenergic receptor, bradykinin receptor, etc.), ion channel proteins, intracellular attachment proteins, cellular adhesion proteins, and integrins. In certain aspects, the effected G-Protein Coupled Receptor (GPCR) interacts with a G protein a subunit (e.g., Gαₛ, Gαᵢ, Gα_{q}, and Gα₁₂).

In particular aspects, the electrokinetically altered aqueous fluids are suitable to modulate intracellular signal transduction, comprising modulation of a calcium dependant cellular messaging pathway or system (e.g., modulation of phospholipase C activity, or modulation of adenylate cyclase (AC) activity).

In particular aspects, the electrokinetically altered aqueous fluids are characterized by various biological activities (e.g., regulation of cytokines, receptors, enzymes and other proteins and intracellular signaling pathways) described in the working Examples and elsewhere herein.

In particular aspects, the electrokinetically altered aqueous fluids inhibit the DEP-induced cell surface-bound MMP9 levels in bronchial epithelial cells (BEC) as shown in working Examples herein.

In particular aspects, the biological effects of the electrokinetically altered aqueous fluids are inhibited by diphtheria toxin, indicating that beta blockade, GPCR blockade and Ca channel blockade affects the activity of the electrokinetically altered aqueous fluids (e.g., on regulatory T cell function) as shown in working Examples herein.

In particular aspects, the physical and biological effects (e.g., the ability to alter cellular membrane structure or function sufficient to provide for modulation of intracellular signal transduction) of the electrokinetically altered aqueous fluids persists for at least two, at least three, at least four, at least five, at least 6 months, or longer periods, in a closed container (e.g., closed gas-tight container).

Therefore, further aspects provide said electrokinetically-generated solutions and methods of producing an electrokinetically altered oxygenated aqueous fluid or solution, comprising: providing a flow of a fluid material between two spaced surfaces in relative motion and defining a mixing volume therebetween, wherein the dwell time of a single pass of the flowing fluid material within and through the mixing volume is greater than 0.06 seconds or greater than 0.1 seconds; and introducing oxygen (0₂) into the flowing fluid material within the mixing volume under conditions suitable to dissolve at least 20 ppm, at least 25 ppm, at least 30, at least 40, at least 50, or at least 60 ppm oxygen into the material, and electrokinetically alter the fluid or solution. In certain aspects, the oxygen is infused into the material in less than 100 milliseconds, less than 200 milliseconds, less than 300 milliseconds, or less than 400 milliseconds. In particular embodiments, the ratio of surface area to the volume is at least 12, at least 20, at least 30, at least 40, or at least 50.

Yet further aspects, provide a method of producing an electrokinetically altered oxygenated aqueous fluid or solution, comprising: providing a flow of a fluid material between two spaced surfaces defining a mixing volume therebetween; and introducing oxygen into the flowing material within the mixing volume under conditions suitable to infuse at least 20 ppm, at least 25 ppm, at least 30, at least 40, at least 50, or at least 60 ppm oxygen into the material in less than 100 milliseconds, less than 200 milliseconds, less than 300 milliseconds, or less than 400 milliseconds. In certain aspects, the dwell time of the flowing material within the mixing volume is greater than 0.06 seconds or greater than 0.1 seconds. In particular embodiments, the ratio of surface area to the volume is at least 12, at least 20, at least 30, at least 40, or at least 50.

Additional embodiments provide a method of producing an electrokinetically altered oxygenated aqueous fluid or solution, comprising use of a mixing device for creating an output mixture by mixing a first material and a second material, the device comprising: a first chamber configured to receive the first material from a source of the first material; a stator; a rotor having an axis of rotation, the rotor being disposed inside the stator and configured to rotate about the axis of rotation therein, at least one of the rotor and stator having a plurality of through-holes; a mixing chamber defined between the rotor and the stator, the mixing chamber being in fluid communication with the first chamber and configured to receive the first material therefrom, and the second material being provided to the mixing chamber via the plurality of through-holes formed in the one of the rotor and stator; a second chamber in fluid communication with the mixing chamber and configured to receive the output material therefrom; and a first internal pump housed inside the first chamber, the first internal pump being configured to pump the first material from the first chamber into the mixing chamber. In certain aspects, the first internal pump is configured to impart a circumferential velocity into the first material before it enters the mixing chamber.

Further embodiments provide a method of producing an electrokinetically altered oxygenated aqueous fluid or solution, comprising use of a mixing device for creating an output mixture by mixing a first material and a second material, the device comprising: a stator; a rotor having an axis of rotation, the rotor being disposed inside the stator and configured to rotate about the axis of rotation therein; a mixing chamber defined between the rotor and the stator, the mixing chamber having an open first end through which the first material enters the mixing chamber and an open second end through which the output material exits the mixing chamber, the second material entering the mixing chamber through at least one of the rotor and the stator; a first chamber in communication with at least a majority portion of the open first end of the mixing chamber; and a second chamber in communication with the open second end of the mixing chamber.

Additional aspects provide an electrokinetically altered oxygenated aqueous fluid or solution made according to any of the above methods. In particular aspects the administered inventive electrokinetically-altered fluids comprise charge-stabilized oxygen-containing nanostructures in an amount sufficient to provide modulation of at least one of cellular membrane potential and cellular membrane conductivity. In certain embodiments, the electrokinetically-altered fluids are superoxygenated (e.g., RNS-20, RNS-40 and RNS-60, comprising 20 ppm, 40 ppm and 60 ppm dissolved oxygen, respectively, in standard saline). In particular embodiments, the electrokinetically-altered fluids are not-superoxygenated (e.g., RNS-10 or Solas, comprising 10 ppm (e.g., approx. ambient levels of dissolved oxygen in standard saline). In certain aspects, the salinity, sterility, pH, etc., of the inventive electrokinetically-altered fluids is established at the time of electrokinetic production of the fluid, and the sterile fluids are administered by an appropriate route. Alternatively, at least one of the salinity, sterility, pH, etc., of the fluids is appropriately adjusted (e.g., using sterile saline or appropriate diluents) to be physiologically compatible with the route of administration prior to administration of the fluid. Preferably, diluents and/or saline solutions and/or buffer compositions used to adjust at least one of the salinity, sterility, pH, etc., of the fluids are also electrokinetic fluids, or are otherwise compatible therewith.

In particular aspects, the inventive electrokinetically-altered fluids comprise saline (e.g., one or more dissolved salt(s); e.g., alkali metal based salts (Li, Na, K, Rb, Cs, etc.) or alkaline earth based salts (e.g., Mg, Ca), etc., with any suitable anion components). Particular aspects comprise mixed salt based electrokinetic fluids (e.g., Na, K, Ca, Mg, etc., in various combinations and concentrations). In particular aspects, the inventive electrokinetically-altered fluids comprise standard saline (e.g., approx. 0.9% NaCl, or about 0.15 M NaCl). In particular aspects, the inventive electrokinetically-altered fluids comprise saline at a concentration of at least 0.0002 M, at least 0.0003 M, at least 0.001 M, at least 0.005 M, at least 0.01 M, at least 0.015 M, at least 0.1 molar, at least 0.15 M, or at least 0.2 M. In particular aspects, the conductivity of the inventive electrokinetically-altered fluids is at least 10 μS/cm, at least 40 μS/cm, at least 80 μS/cm, at least 100 μS/cm, at least 150 μS/cm, at least 200 μS/cm, at least 300 μS/cm, or at least 500 μS/cm, at least 1 mS/cm, at least 5, mS/cm, 10 mS/cm, at least 40 mS/cm, at least 80 mS/cm, at least 100 mS/cm, at least 150 mS/cm, at least 200 mS/cm, at least 300 mS/cm, or at least 500 mS/cm. In particular aspects, any salt may be used in preparing the inventive electrokinetically-altered fluids, provided that they allow for formation of biologically active salt-stabilized nanostructures (e.g., salt-stabilized oxygen-containing nanostructures) as disclosed herein.

According to particular aspects, the biological effects of the inventive fluid compositions comprising charge-stabilized gas-containing nanostructures can be modulated (e.g., increased, decreased, tuned, etc.) by altering the ionic components of the fluids as, for example, described above, and/or by altering the gas component of the fluid. In preferred aspects, oxygen is used in preparing the inventive electrokinetic fluids. In additional aspects mixtures of oxygen along with at least one other gas selected from Nitrogen, Oxygen, Argon, Carbon dioxide, Neon, Helium, krypton, hydrogen and Xenon.

Given the teachings and assay systems disclosed herein (e.g., cell-based cytokine assays, patch-clamp assays, etc.) one of skill in the art will readily be able to select appropriate salts and concentrations thereof to achieve the biological activities disclosed herein.

TABLE 1
Exemplary cations and anions.

| | | |
|---|---|---|
| Name | Formula | Other name(s) |

Common Cations:

| | | |
|---|---|---|
| Aluminum | Al⁺³ | |
| Ammonium | NH₄⁺ | |
| Barium | Ba⁺² | |
| Calcium | Ca⁺² | |
| Chromium(II) | Cr⁺² | Chromous |
| Chromium(III) | Cr⁺³ | Chromic |
| Copper(I) | Cu⁺ | Cuprous |
| Copper(II) | Cu⁺² | Cupric |
| Iron(II) | Fe⁺² | Ferrous |
| Iron(III) | Fe⁺³ | Ferric |
| Hydrogen | H⁺ | |
| Hydronium | H₃O⁺ | |
| Lead(II) | Pb⁺² | |
| Lithium | Li⁺ | |
| Magnesium | Mg⁺² | |
| Manganese(II) | Mn⁺² | Manganous |
| Manganese(III) | Mn⁺³ | Manganic |
| Mercury(I) | Hg₂⁺² | Mercurous |
| Mercury(II) | Hg⁺² | Mercuric |
| Nitronium | NO₂⁺ | |
| Potassium | K⁺ | |
| Silver | Ag⁺ | |
| Sodium | Na⁺ | |
| Strontium | Sr⁺² | |
| Tin(II) | Sn⁺² | Stannous |
| Tin(IV) | Sn⁺⁴ | Stannic |
| Zinc | Zn⁺² | |

Common Anions:

| | | | |
|---|---|---|---|
| Simple ions: | | | |
| Hydride | H⁻ | Oxide | O²⁻ |
| Fluoride | F⁻ | Sulfide | S²⁻ |
| Chloride | Cl⁻ | Nitride | N³⁻ |
| Bromide | Br⁻ | | |
| Iodide | I⁻ | | |
| Oxoanions: | | | |
| Arsenate | AsO₄³⁻ | Phosphate | PO₄³⁻ |
| Arsenite | AsO₃³⁻ | Hydrogen phosphate | HPO₄²⁻ |
| | | Dihydrogen phosphate | H₂PO₄⁻ |
| Sulfate | SO₄²⁻ | Nitrate | NO₃⁻ |
| Hydrogen sulfate | HSO₄⁻ | Nitrite | NO₂⁻ |
| Thiosulfate | S₂O₃²⁻ | | |
| Sulfite | SO₃²⁻ | | |
| Perchlorate | ClO₄⁻ | Iodate | IO₃⁻ |
| Chlorate | ClO₃⁻ | Bromate | BrO₃⁻ |
| Chlorite | ClO₂⁻ | | |
| Hypochlorite | OCl⁻ | Hypobromite | OBr⁻ |
| Carbonate | CO₃²⁻ | Chromate | CrO₄²⁻ |
| Hydrogen carbonate | HCO₃⁻ | Dichromate | Cr₂O₇²⁻ |
| or Bicarbonate | | | |

Anions from Organic Acids:

| | | | |
|---|---|---|---|
| Acetate | CH₃COO⁻ | formate | HCOO⁻ |

Others:

| | | | |
|---|---|---|---|
| Cyanide | CN⁻ | Amide | NH₂⁻ |
| Cyanate | OCN⁻ | Peroxide | O₂²⁻ |
| Thiocyanate | SCN⁻ | Oxalate | C₂O₄²⁻ |
| Hydroxide | OH⁻ | Permanganate | MnO₄⁻ |

TABLE 2
Exemplary cations and anions.

| | | |
|---|---|---|
| Formula | Charge | Name |

Monoatomic Cations

| | | |
|---|---|---|
| H⁺ | 1+ | hydrogen ion |
| Li⁺ | 1+ | lithium ion |
| Na⁺ | 1+ | sodium ion |
| K⁺ | 1+ | potassium ion |
| Cs⁺ | 1+ | cesium ion |
| Ag⁺ | 1+ | silver ion |
| Mg²⁺ | 2+ | magnesium ion |
| Ca²⁺ | 2+ | calcium ion |
| Sr²⁺ | 2+ | strontium ion |
| Ba²⁺ | 2+ | barium ion |
| Zn²⁺ | 2+ | zinc ion |
| Cd²⁺ | 2+ | cadmium ion |
| Al³⁺ | 3+ | aluminum ion |

Polyatomic Cations

| | | |
|---|---|---|
| NH₄⁺ | 1+ | ammonium ion |
| H₃O⁺ | 1+ | hydronium ion |

Multivalent Cations

| | | |
|---|---|---|
| Cr²⁺ | 2 | chromium(II) or chromous ion |
| Cr³⁺ | 3 | chromium(III)or chromic ion |
| Mn²⁺ | 2 | manganese(II) or manganous ion |
| Mn⁴⁺ | 4 | manganese(IV) ion |
| Fe²⁺ | 2 | iron(II) or ferrous ion |
| Fe³⁺ | 3 | iron(III) or ferric ion |
| Co²⁺ | 2 | cobalt(II) or cobaltous ion |
| Co³⁺ | 3 | cobalt(II) or cobaltic ion |
| Ni²⁺ | 2 | nickel(II) or nickelous ion |
| Ni³⁺ | 3 | nickel(III) or nickelic ion |
| Cu⁺ | 1 | copper(I) or cuprous ion |
| Cu²⁺ | 2 | copper(II) or cupric ion |
| Sn²⁺ | 2 | tin(II) or atannous ion |
| Sn⁴⁺ | 4 | tin(IV) or atannic ion |
| Pb²⁺ | 2 | lead(II) or plumbous ion |
| Pb⁴⁺ | 4 | lead(IV) or plumbic ion |

Monoatomic Anions

| | | |
|---|---|---|
| H⁻ | 1− | hydride ion |
| F⁻ | 1− | fluoride ion |
| Cl⁻ | 1− | chloride ion |
| Br⁻ | 1− | bromide ion |
| I⁻ | 1− | iodide ion |
| O²⁻ | 2− | oxide ion |
| S²⁻ | 2− | sulfide ion |
| N³⁻ | 3− | nitride ion |

Polyatomic Anions

| | | |
|---|---|---|
| OH⁻ | 1− | hydroxide ion |
| CN⁻ | 1− | cyanide ion |
| SCN⁻ | 1− | thiocyanate ion |
| C₂H₃O₂⁻ | 1− | acetate ion |
| ClO⁻ | 1− | hypochlorite ion |
| ClO₂⁻ | 1− | chlorite ion |
| ClO₃⁻ | 1− | chlorate ion |
| ClO₄⁻ | 1− | perchlorate ion |
| NO₂⁻ | 1− | nitrite ion |
| NO₃⁻ | 1− | nitrate ion |
| MnO₄²⁻ | 2− | permanganate ion |
| CO₃²⁻ | 2− | carbonate ion |
| C₂O₄²⁻ | 2− | oxalate ion |
| CrO₄²⁻ | 2− | chromate ion |
| Cr₂O₇²⁻ | 2− | dichromate ion |
| SO₃²⁻ | 2− | sulfite ion |
| SO₄²⁻ | 2− | sulfate ion |
| PO₃³⁻ | 3− | phosphite ion |
| PO₄³⁻ | 3− | phosphate ion |

The present disclosure sets forth novel gas-enriched fluids, including, but not limited to gas-enriched ionic aqueous solutions, aqueous saline solutions (e.g., standard aqueous saline solutions, and other saline solutions as discussed herein and as would be recognized in the art, including any physiological compatible saline solutions), cell culture media (e.g., minimal medium, and other culture media).

### Cardiovascular Diseases and Related Conditions

Cardiovascular diseases are a large class of diseases that involve the heart or blood vessels (arteries and veins). Cardiovascular diseases include, but are not limited to cardiac arrhythmia, vascular disease, myocardial infarction, congestive heart failure, myocarditis, atherosclerosis, and restenosis. These conditions have similar causes, mechanisms, and treatments. Most cardiovascular diseases share common risk factors, including inflammation, high cholestrol, and obesity. Inflammatory biomarkers, including C-reactive protein, interleukin 6 (IL-6), and interleukin 8 (IL-8), have been associated with cardiovascular disease. In addition, matrix metalloproteinases recently have been found to have a role in cardiovascular disease.

Cardiac arrhythmia is a term for any of a large and heterogeneous group of conditions in which there is abnormal electrical activity in the heart. The heart beat may be too fast or too slow, and may be regular or irregular. Some arrhythmias are life-threatening medical emergencies that can result in cardiac arrest and sudden death. Others cause symptoms such as an abnormal awareness of heart beat (palpitations), and may be merely annoying. These palpitations have also been known to be caused by atrial/ventricular fibrillation, wire faults, and other technical or mechanical issues in cardiac pacemakers/defibrillators. Still others may not be associated with any symptoms at all, but may predispose the patient to potentially life threatening stroke or embolism. Treatments for cardiac arrhythmia include a group of drugs called antiarrhythmic agents which are used to suppress fast rhythms of the heart (cardiac arrhythmias), such as atrial fibrillation, atrial flutter, ventricular tachycardia, and ventricular fibrillation. There are five main classes of antiarrhythmic agents. Class I agents, including but not limited to, quinidine, procainamide, disopyramide, lidocaine, phenytoin, mexiletine, flecainide, propafenone, and moricizine, interfere with the sodium (Na⁺) channel. Class II agents (e.g., propranolol, esmolol, timolol, metoprolol, atenolol, and bisoprolo) are anti-sympathetic nervous system agents, most of which are beta blockers. Class III agents (e.g., amiodarone, sotalol, ibutilide, dofetilide, dronedarone and E-4031) affect potassium (K⁺) efflux. Class IV agents (e.g., verapamil and diltiazem) affect calcium channels and the AV node. Class V agents (e.g., adenosine, digoxin and magnesium sulfate) work by other or unknown mechanisms. In addition, since some arrhythmias promote blood clotting within the heart, and increase risk of embolus and stroke, anticoagulant medications, (e.g., warfarin and heparins) and anti-platelet drugs such as aspirin frequently are used to reduce the risk of clotting.

Vascular disease is a pathological state of large and medium sized muscular arteries and is triggered by endothelial cell dysfunction. Because of factors like pathogens, oxidized LDL particles and other inflammatory stimuli endothelial cells become activated. This leads to change in their characteristics: endothelial cells start to excrete cytokines and chemokines and express adhesion molecules on their surface. This in turn results in recruitment of white blood cells (monocytes and lymphocytes), which can infiltrate the blood vessel wall. Stimulation of smooth muscle cell layer with cytokines produced by endothelial cells and recruited white blood cells causes smooth muscle cells to proliferate and migrate towards the blood vessel lumen. The process causes thickening of the vessel wall, forming a plaque consisting of proliferating smooth muscle cells, macrophages and various types of lymphocytes. This plaque result in obstructed blood flow leading to diminished amounts of oxygen and nutrients reaching the target organ. In the final stages, the plaque may also rupture causing the formation of clots, and as a result strokes.

Myocardial infarction (MI) or acute myocardial infarction (AMI), commonly known as a heart attack, is the interruption of blood supply to part of the heart, causing myocardial cellular death. This is most commonly due to occlusion of a coronary artery following the rupture of a vulnerable atherosclerotic plaque, which is an unstable collection of lipids and white blood in the wall of an artery. The resulting ischemia and oxygen shortage, if left untreated for a sufficient period of time, can cause damage or death (infarction) of heart muscle tissue.

Classical symptoms of acute myocardial infarction include sudden chest pain (typically radiating to the left arm or left side of the neck), shortness of breath, nausea, vomiting, palpitations, sweating, and anxiety (often described as a sense of impending doom). Women may experience fewer typical symptoms than men, most commonly shortness of breath, weakness, a feeling of indigestion, and fatigue. Approximately one quarter of all myocardial infarctions are silent, without chest pain or other symptoms.

Among the diagnostic tests available to detect heart muscle damage are an electrocardiogram (ECG), chest X-ray, and various blood tests. The most often used markers are the creatine kinase-MB (CK-MB) fraction and the troponin levels. Immediate treatment for suspected acute myocardial infarction includes oxygen, aspirin, and sublingual nitroglycerin.

Patients are usually commenced on several long-term medications post-MI, with the aim of preventing secondary cardiovascular events such as further myocardial infarctions, congestive heart failure or cerebrovascular accident (CVA). Medications useful in preventing secondary cardiovascular events include, but are not limited to: antiplatelet drug therapy (e.g., aspirin and clopidogrel), beta blockers (e.g., metoprolol and carvedilol), angiotensin-converting enzyme (ACE) inhibitors (e.g., captopril, zofenopril, enalapril, ramipril, quinapril, perindopril, lisinopril, benazepril, fosinopril, casokinins and lactokinins), statins (e.g., atorvastatin, cerivastatin, fluvastatin, lovastatin, pitavastatin, mevastatin, pravastatin, rosuvastatin, and simvastatin), aldosterone antagonist agents (e.g., eplerenone and spironolactone) and omega-3 fatty acids.

Congestive heart failure (CHF), or heart failure, is a condition in which the heart is restricted from pumping enough blood to the body's other organs. This can result from narrowed arteries that supply blood to the heart muscle (e.g., coronary artery disease), past myocardial infarction having scar tissue that interferes with the heart muscle's normal work, high blood pressure, heart valve disease due to past rheumatic fever or other causes, cardiomyopathy, congenital heart defects, endocarditis and/or myocarditis. As blood flow out of the heart slows, blood returning to the heart through the veins backs up, causing congestion in the tissues, and often causing edema. Sometimes fluid collects in the lungs and interferes with breathing, causing shortness of breath, especially when a person is lying down. Treatment for CHF includes rest, proper diet, modified daily activities and pharmaceuticals, which includes, but is not limited to beta blockers (e.g., metoprolol and carvedilol), ACE inhibitors (e.g., captopril, zofenopril, enalapril, ramipril, quinapril, perindopril, lisinopril, benazepril, fosinopril, casokinins and lactokinins), digitalis, diuretics, and vasodilators. ACE inhibitors and vasodilators expand blood vessels and decrease resistance. Beta blockers can improve how well the heart's left lower chamber (left ventricle) pumps. Digitalis increases the pumping action of the heart muscle; while diuretics help the body eliminate excess salt and water.

Myocarditis is inflammation of heart muscle (myocardium). It resembles a heart attack but coronary arteries are not blocked. Myocarditis is most often due to infection by common viruses, such as parvovirus B19, less commonly non-viral pathogens such as Borrelia burgdorferi (Lyme disease) or Trypanosoma cruzi, or as a hypersensitivity response to drugs.http://en.wikipedia.orq/wiki/Myocarditis—cite note-Cooper-0#cite note-Cooper-0 The central feature of myocarditis is an infection of the heart, with an inflammatory infiltrate, and damage to the myocardium, without the blockage of coronary arteries or other common non-infectious causes. Myocarditis may or may not include necrosis of heart tissue. Myocarditis can be an autoimmune reaction, due to infection of certain agents, because, for example, Streptococcal M protein and coxsackievirus B have epitopes that are immunologically similar to cardiac myosin. After the agent is cleared from the body, the immune system can attack cardiac myosin.

Symptoms of myocarditis vary widely. Myocarditis can cause a mild disease without any symptoms that resolves itself, or it may cause chest pain, heart failure, or sudden death. Treatment of myocarditis can include digoxin, diuretics, inotropes (e.g., Milrinone) and ACE inhibitors (e.g., Captopril, Lisinopril).

Atherosclerosis is a condition in which an artery wall thickens as the result of a build-up (called plaques) of fatty materials such as cholesterol. In particular, this syndrome affecting arterial blood vessels is a chronic inflammatory response in the walls of arteries, in large part due to the accumulation of macrophage white blood cells and promoted by low-density lipoproteins without adequate removal of fats and cholesterol from the macrophages by functional high density lipoproteins (HDL). It is commonly referred to as a hardening or furring of the arteries.

The following terms are similar, yet distinct, in both spelling and meaning, and can be easily confused: arteriosclerosis, arteriolosclerosis, and atherosclerosis. Arteriosclerosis is a general term describing any hardening (and loss of elasticity) of medium or large arteries; arteriolosclerosis is any hardening (and loss of elasticity) of arterioles (small arteries); atherosclerosis is a hardening of an artery specifically due to an atheromatous plaque. The term atherogenic is used for substances or processes that cause atherosclerosis.

Atherosclerosis, though typically asymptomatic for decades, eventually produces two main problems: First, the atheromatous plaques, though long compensated for by artery enlargement eventually lead to plaque ruptures and clots inside the artery lumen over the ruptures. The clots heal and usually shrink but leave behind stenosis of the artery (both locally and in smaller downstream branches), or worse, complete closure, and, therefore, an insufficient blood supply to the tissues and organ it feeds. Second, if the compensating artery enlargement process is excessive, then a net aneurysm can occur. Since atherosclerosis is a body-wide process, these events can occur in the arteries to the brain, heart, intestines, kidneys, legs, etc.

Treatment for atherosclerosis includes, but is not limited to, beta blockers (e.g., metoprolol and carvedilol), ACE inhibitors (e.g., captopril, zofenopril, enalapril, ramipril, quinapril, perindopril, lisinopril, benazepril, fosinopril, casokinins and lactokinins), statins (e.g., atorvastatin, cerivastatin, fluvastatin, lovastatin, pitavastatin, mevastatin, pravastatin, rosuvastatin, and simvastatin), diuretics, and dietary supplements (e.g., folic acid, niacin, omega 3 fatty acids, and vitamin C).

Restenosis is the reoccurrence of stenosis, a narrowing of a blood vessel, leading to restricted blood flow. Restenosis usually pertains to an artery or other large blood vessel that has become narrowed, received treatment to clear the blockage and subsequently become renarrowed. It can be defined as a reduction in the circumference of the lumen of 50% or more, and had a high incidence rate (25-50%) in patients who had undergone balloon angioplasty, with the majority of patients needing further angioplasty within 6 months. Restenosis treatments include, but are not limited to angioplasty, brachytherapy, and intracoronary radiation.

### Inflammation

Inflammation is known to play a role in cardiovascular diseases (see, e.g., Jialal and Devaraj S, “Inflammation and atherosclerosis: the value of the high-sensitivity C-reactive protein assay as a risk marker.” Am J Clin Pathol (2001) 116 Suppl:S108-15; Zairis M, et al, “C-reactive protein and multiple complex coronary artery plaques in patients with primary unstable angina.” Atherosclerosis (2002) 164(2):355; Lowe GD, “The relationship between infection, inflammation, and cardiovascular disease: an overview.” Ann Peridontol (2001) 6(1):1-8; Rifai and Ridker, “Inflammatory markers and coronary heart disease.” Curr Opin Lipidol (2002) 13(4):383-9; Bermudez and Ridker, “C-reactive protein, statins, and the primary prevention of atherosclerotic cardiovascular disease.” Prey Cardiol (2002) 5(1):42-6; Blake and Ridker, “Inflammatory mechanisms in atherosclerosis: from laboratory evidence to clinical application.” Ital Heart J (2001) 2(11):796-800; Pradhan AD, et al, “Inflammatory biomarkers, hormone replacement therapy, and incident coronary heart disease: prospective analysis from the Women's Health Initiative observational study”. JAMA (2002) 288(8):980-7; Ridker P M, et al. “Inflammation, aspirin, and the risk of cardiovascular disease in apparently healthy men.” NEJM (1997) 336(14):973-9; “Interleukin 8 and cardiovascular disease” Cardiovasc Res (2009) doi: 10.1093/cvr/cvp241).

Inflammation may occur as a defensive response to invasion of the subject by foreign material, particularly of microbial origin. Additionally, mechanical trauma, toxins, and neoplasia may induce inflammatory responses. The accumulation and subsequent activation of leukocytes are central events in the pathogenesis of most forms of inflammation. Inflammation deficiencies can compromise the host, leaving it susceptible to worsening infection or trauma. Excessive inflammation, such as prolonged inflammatory responses, may lead to inflammatory diseases including but not limited to diabetes, cardiovascular disease (e.g, arteriosclerosis, cardiac arrhythmia, vascular disease, myocardial infarction, congestive heart failure, myocarditis, atherosclerosis, and restenosis) macular degeneration, cataracts, chronic skin disorders, reperfusion injury, and cancer, to post-infectious syndromes such as in infectious meningitis, rheumatic fever, and to rheumatic diseases such as systemic lupus erythematosus and rheumatoid arthritis. These diseases affect millions of people worldwide every year, and lead to increased mortality and morbidity. The commonality of the inflammatory response in these varied disease processes makes its regulation a major element in the prevention, or treatment of human disease.

Overproduction of pro-inflammatory cytokines has been implicated in the pathogenesis of numerous inflammatory and autoimmune diseases. Secretion of TNFα is a primary event in the initiation of the inflammatory cascade (Brennan F. M., et. al. Lancet, 1989, 2:244-7; Haworth C, et. al. Eur. J. Immunol. 1991, 21:2575-2579) and directly contributes to the initiation and maintenance of these diseases. Other cytokines also play a role, including interleukin 1β (IL-1β), IL-6, IL-8, IL-12 nitric oxide (NO), IFN-γ, granulocyte colony stimulating factor (G-CSF), granulocyte macrophage-colony stimulating factor (GM-CSF), and IL-10. Certain of these cytokines (e.g. IL-8) may increase or exacerbate an inflammatory response, while others (e.g. IL-10) may decrease or alleviate the inflammatory response.

Cells of the immune system, macrophages in particular, secrete many of these cytokines in response to activating stimuli. Target cells of the cytokines may be localized in any body compartment and may act via long-distance mechanisms, or may act on neighboring cells. Thus, cytokines may regulate inflammation in a localized or systemic manner.

### Link Between Cardiovascular Diseases and Inflammation

A chronic inflammatory state, as evidenced by elevated C-reactive protein, results in significant damage to the arterial system, including thrombus formation, plaque rupture, and embolization. C-reactive protein (CRP) is a sensitive but non-specific marker for inflammation. Elevated CRP blood levels, especially measured with high sensitivity assays, can predict the risk of MI, as well as stroke and development of diabetes. Moreover, some drugs for MI have been shown to reduce CRP levels. However, whether CRP plays a direct role in atherosclerosis or cardiovasculardisease remains uncertain.

Studies on C-reactive protein indicate that cholesterol-filled plaques in blood vessels may not pose any real danger unless they are affected by inflammation. Inflammation weakens plaques, making them more vulnerable to bursting or pinching off a clot that can then block coronary vessels (Jialal and Devaraj S, “Inflammation and atherosclerosis: the value of the high-sensitivity C-reactive protein assay as a risk marker.” Am J Clin Pathol 2001 December; 116 Suppl:S108-15; Zairis M, et al, “C-reactive protein and multiple complex coronary artery plaques in patients with primary unstable angina.” Atherosclerosis 2002 October; 164(2):355; Lowe GD, “The relationship between infection, inflammation, and cardiovascular disease: an overview.” Ann Peridontol 2001 December; 6(1):1-8; all of which are herein incorporated by reference in their entirety, including their teachings concerning the role of inflammation in cardiovascular disease). In particular, a series of studies indicates that 25 to 35 million Americans have total cholesterol within normal range but above-average levels of inflammation within their cardiovascular systems. This inflammation has significant impact on heart disease risk (Rifai and Ridker, “Inflammatory markers and coronary heart disease.” Curr Opin Lipidol 2002 August; 13(4):383-9; Bermudez and Ridker, “C-reactive protein, statins, and the primary prevention of atherosclerotic cardiovascular disease.” Prev Cardiol 2002 Winter;5(1):42-6; Blake and

Ridker, “Inflammatory mechanisms in atherosclerosis: from laboratory evidence to clinical application.” Ital Heart J 2001 November; 2(11):796-800; all of which are herein incorporated by reference in their entirety, including their teachings concerning the role of inflammation in cardiovascular disease). In addition, the Women's Health Study, which involved 39,876 healthy postmenopausal women, supported the C-reactive protein (and thus chronic inflammation) link to cardiovascular disease (Pradhan A D, et al, “Inflammatory biomarkers, hormone replacement therapy, and incident coronary heart disease: prospective analysis from the Women's Health Initiative observational study”. JAMA 2002 Aug. 28; 288(8):980-7; which is herein incorporated by reference in its entirety, including its teachings concerning the role of inflammation in cardiovascular disease). Those with the highest levels of C-reactive protein had five times the risk of developing cardiovascular disease and seven times the risk of having a heart attack or stroke compared to subjects with the lowest levels. Interestingly, C-reactive protein levels predicted risk of these events even in women who appeared to have no other pertinent risk factors. In addition, the Physicians' Health Study, which evaluated C-reactive protein levels and heart disease risk in 22,000 initially healthy men, supports the relationship between inflammation and heart attack (Ridker P M, et al. “Inflammation, aspirin, and the risk of cardiovascular disease in apparently healthy men.” NEJM 1997 Apr. 3; 336(14):973-9; which is herein incorporated by reference in its entirety, including its teachings concerning the role of inflammation in cardiovascular disease).

In addition, certain pro-inflammatory cytokines and chemokines known to have a role in cardiovascular diseases, including but not limited to atherosclerosis. In particular, interleukin 8 has been shown to be involved in the establishment and preservation of the inflammatory micro-environment of the insulted vascular wall (for a review: “Interleukin 8 and cardiovascular disease” Cardiovasc Res (2009), doi: 10.1093/cvr/cvp241; which is herein incorporated by reference in its entirety, including its teachings concerning the role of IL-8 in cardiovascular disease).

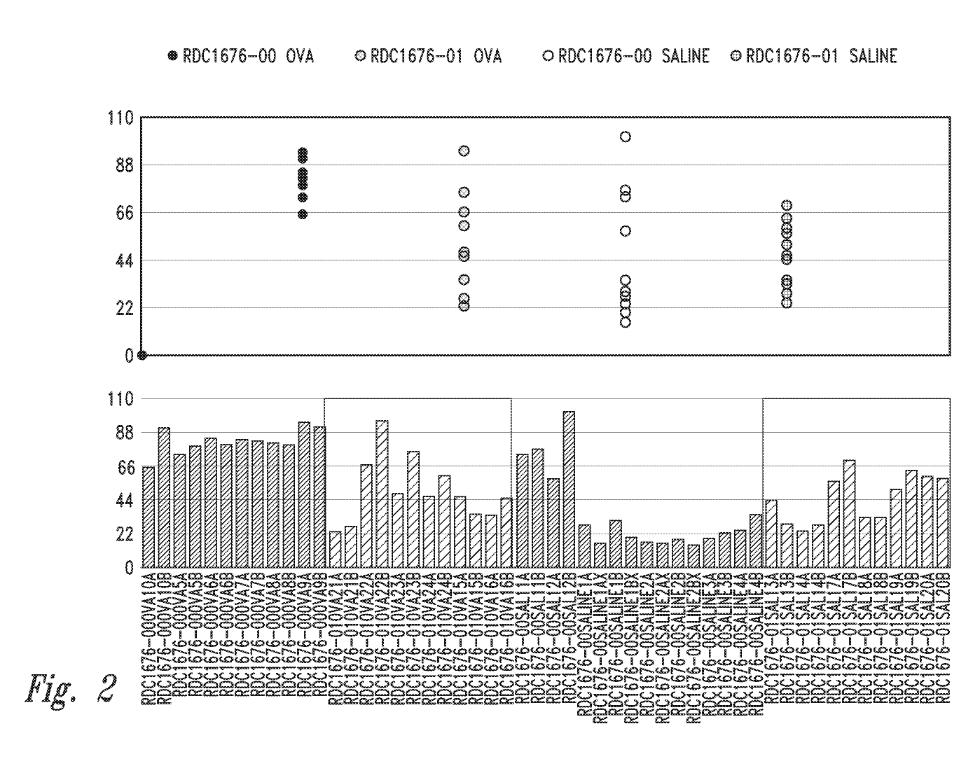

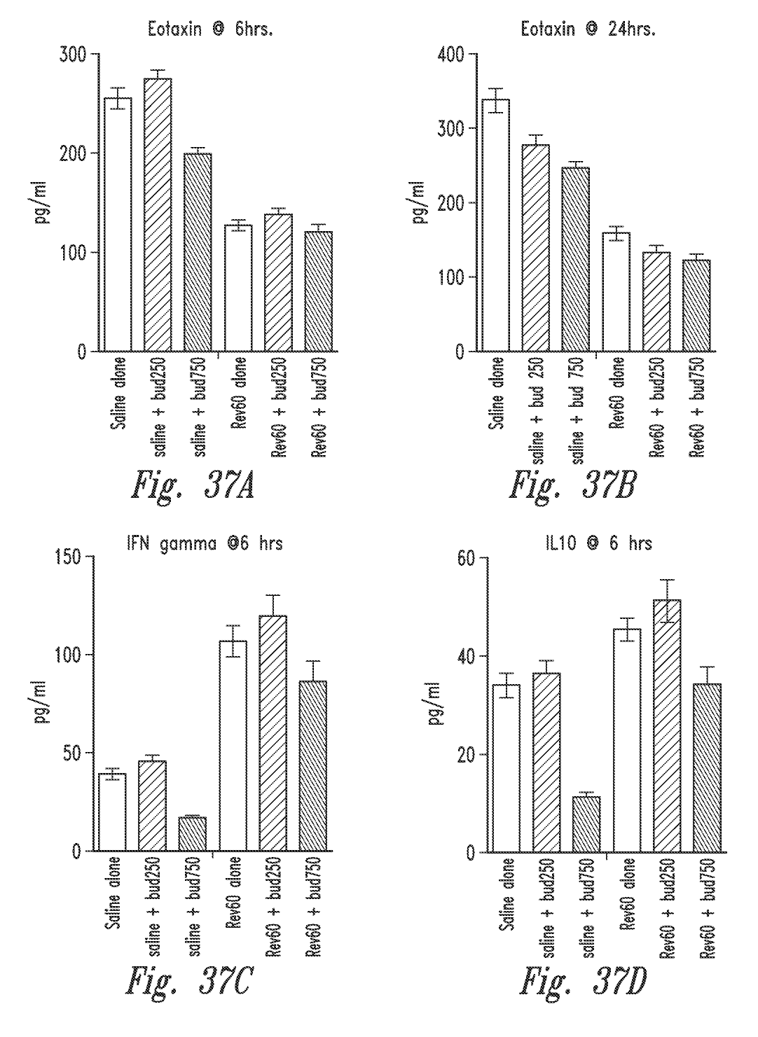

As can be seen from FIG. 2 and FIGS. 37A and B, the electrokinetically altered aqueous fluids reduced the levels of the pro-inflammatory cytokine IL-6 and the pro-inflammatory chemokines IL-8 and Eotaxin when compared to the control fluid. Thus, according to certain embodiments, the inventive fluid alleviates many of the symptoms and/or conditions of several cardiovascular diseases by reducing the levels of pro-inflammatory cytokines and chemokines which thereby limits inflammation.

### Metalloproteinases

Metalloproteinases are a superfamily of proteinases (enzymes) classified into families and subfamilies as described, for example, in N. M. Hooper FEBS Letters 354:1-6, 1994. Examples of metalloproteinases include the matrix metalloproteinases (MMPs) such as the collagenases (MMP1, MMP8, MMP13), the gelatinases (MMP2, MMP9), the stromelysins (MMP3, MMP10, MMP II), matrilysin (MMPI), metalloelastase (MMP12), enamelysin (MMP19), the MT-MMPs (MMP14, MMP15, MMP16, MMP17); the reprolysin or adamalysin or MDC family which includes the secretases and sheddases such as TNF converting enzymes (ADAM10 and TACE); the astacin family which include enzymes such as procollagen processing proteinase (PCP); and other metalloproteinases such as aggrecanase, the endothelin converting enzyme family and the angiotensin converting enzyme family. Collectively, the metalloproteinases are known to cleave a broad range of matrix substrates such as collagen, proteoglycan and fibronectin. Metalloproteinases are implicated in the processing, or secretion, of biological important cell mediators, such as tumour necrosis factor (TNF); and the post translational proteolysis processing, or shedding, of biologically important membrane proteins, such as the low affinity IgE receptor CD23 (see, e.g., N. M. Hooper et al., Biochem. J. 321:265-279, 1997).

Not surprisingly, therefore, metalloproteinases are believed to be important in many physiological disease processes that involve tissue remodeling (e.g., embryonic development, bone formation, uterine remodelling during menstruation, etc.). Moreover, inhibition of the activity of one or more metalloproteinases may well be of benefit in these diseases or conditions, for example: various inflammatory and allergic diseases such as, inflammation of the joint (especially rheumatoid arthritis, osteoarthritis and gout), inflammation of the gastro-intestinal tract (especially inflammatory bowel disease, ulcerative colitis and gastritis), inflammation of the skin (especially psoriasis, eczema, dermatitis); in tumour metastasis or invasion; in disease associated with uncontrolled degradation of the extracellular matrix such as osteoarthritis; in bone resorptive disease (such as osteoporosis and Paget's disease); in diseases associated with aberrant angiogenesis; the enhanced collagen remodelling associated with diabetes, periodontal disease (such as gingivitis), corneal ulceration, ulceration of the skin, post-operative conditions (such as colonic anastomosis) and dermal wound healing; demyelinating diseases of the central and peripheral nervous systems (such as multiple sclerosis); Alzheimer's disease; extracellular matrix remodelling observed in cardiovascular diseases such as restenosis and atherosclerosis; asthma; rhinitis; and chronic obstructive pulmonary diseases (COPED).

MMP 12, also known as macrophage elastase or metalloelastase, was initially cloned in the mouse (Shapiro et al., Journal of Biological Chemistry 267: 4664, 1992) and has also been cloned in man by the same group in 1995. MMP 12 is preferentially expressed in activated macrophages, and has been shown to be secreted from alveolar macrophages from smokers (Shapiro et al, 1993, Journal of Biological Chemistry, 268: 23824) as well as in foam cells in atherosclerotic lesions (Matsumoto et al, Am. J. Pathol. 153: 109, 1998). A mouse model of COPD is based on challenge of mice with cigarette smoke for six months, two cigarettes a day six days a week. Wild-type mice developed pulmonary emphysema after this treatment. When MMP12 knock-out mice were tested in this model they developed no significant emphysema, strongly indicating that MMP12 is a key enzyme in the COPD pathogenesis. The role of MMPs such as MMP 12 in COPD (emphysema and bronchitis) is discussed in Anderson and Shinagawa, 1999, Current Opinion in Anti-inflammatory and Immunomodulatory Investigational Drugs 1(1): 29-38. It was recently discovered that smoking increases macrophage infiltration and macrophage-derived MMP-12 expression in human carotid artery plaques (Matetzky S, Fishbein M C et al., Circulation 102:(18), 36-39 Suppl. S, Oct. 31, 2000).

MMP9-(Gelatinase B; 92 kDa-TypeIV Collagenase; 92 kDa Gelatinase) is a secreted protein which was first purified, then cloned and sequenced, in 1989 (S. M. Wilhelm et al., J. Biol. Chem. 264 (29): 17213-17221, 1989; published erratum in J. Biol. Chem. 265 (36): 22570, 1990) (for review of detailed information and references on this protease see T. H. Vu & Z. Werb (1998) (In: Matrix Metalloproteinases, 1998, edited by W. C. Parks & R. P. Mecham, pp. 115-148, Academic Press. ISBN 0-12-545090-7). The expression of MMP9 is restricted normally to a few cell types, including trophoblasts, osteoclasts, neutrophils and macrophages (Vu & Werb, supra). However, the expression can be induced in these same cells and in other cell types by several mediators, including exposure of the cells to growth factors or cytokines. These are the same mediators often implicated in initiating an inflammatory response. As with other secreted MMPs, MMP9 is released as an inactive Pro-enzyme, which is subsequently cleaved to form the enzymatically active enzyme. The proteases required for this activation in vivo are not known. The balance of active MMP9 versus inactive enzyme is further regulated in vivo by interaction with TIMP-1 (Tissue Inhibitor of Metalloproteinases-1), a naturally-occurring protein. TIMP-1 binds to the C-terminal region of MMP9, leading to inhibition of the catalytic domain of MMP9. The balance of induced expression of ProMMP9, cleavage of Pro-to active MMP9 and the presence of TIMP-1 combine to determine the amount of catalytically active MMP9 which is present at a local site. Proteolytically active MMP9 attacks substrates which include gelatin, elastin, and native Type IV and Type V collagens; it has no activity against native Type I collagen, proteoglycans or laminins. There has been a growing body of data implicating roles for MMP9 in various physiological and pathological processes. Physiological roles include the invasion of embryonic trophoblasts through the uterine epithelium in the early stages of embryonic implantation; some role in the growth and development of bones; and migration of inflammatory cells from the vasculature into tissues.

MMP9 release, measured using enzyme immunoassay, was significantly enhanced in fluids and in AM supernatants from untreated asthmatics compared with those from other populations (Am. J. Resp. Cell & Mol. Biol., 5:583-591, 1997). Also, increased MMP9 expression has been observed in certain other pathological conditions, thereby implicating MMP9 in disease processes such as COPD, arthritis, tumour metastasis, Alzheimer's disease, multiple sclerosis, and plaque rupture in atherosclerosis leading to acute coronary conditions such as myocardial infarction (see also WO07087637A3, incorporated herein by reference).

### MMP Inhibitors:

A number of metalloproteinase inhibitors are known (see, for example, the reviews of MMP inhibitors by Beckett R. P. and Whittaker M., 1998, Exp. Opin. Ther. Patents, 8(3):259-282; and by Whittaker M. et al, 1999, Chemical Reviews 99(9):2735-2776). WO 02/074767 discloses hydantoin derivatives of formula that are useful as MMP inhibitors, particularly as potent MMP12 inhibitors. U.S. patent application Ser. No. 11/721,590 (published as 20080032997) discloses a further group of hydantoin derivatives that are inhibitors of metalloproteinases and are of particular interest in inhibiting MMPs such as MMP12 and MMP9. Novel triazolone derivatives for inhibiting MMPs such as MMP12 and MMP9 are disclosed in U.S. patent application Ser. No. 10/593543 (published as 20070219217). Additional MMP12 and MMP9 inhibitors are disclosed in Ser. No. 11/509,490 (published as 20060287338) (see also Ser. No. 10/831265 (published as 20040259896)).

Additionally, two compounds, 4-(4-phenoxyphenylsulfonyl)butane-1,2-dithiol (1) and 5-(4-phenoxyphenylsulfonyl)pentane-1,2-dithiol (2), have been shown to bind selectively and inhibit potently MMP-2 and MMP-9 (Bernardo, et. al (2002) J. Biol. Chem. 277:11201-11207). These two compounds may have significant use in the clinic to inhibit MMP-2 and -9 and therefore lessen inflammation. In addition, the use of certain tetracycline antibiotics (e.g., Minocycline and Doxycycline) at sub-antibiotic levels has been shown to effectively inhibit MMP activity. Certain aspects of this invention include using the inventive fluids in combination with sub-antibiotic levels useful to inhibit MMP.

### Link Between Cardiovascular Disease and Matrix Metalloproteinases

Matrix metalloproteinases (MMPs) have been shown recently to be involved in the pathogenesis of many cardiovascular diseases, including atherosclerosis, restenosis, dilated cardiomyopathy, and myocardial infarction (Creemers, et al., “Matrix Metalloproteinase Inhibition after Myocardial Infarction: a new approach to prevent hear failure?” (2001) Circ Res. 89(3):201-10; Sierevogel et al., (2003) “Matrix metalloproteinases: a therapeutic target in cardiovascular disease.” Curr Pharm Des. 9(13):1033-40; both of which are herein incorporated by reference in their entirety, including for their teachings concerning the role of MMPs in cardiovascular disease). Interestingly, administration of synthetic MMP inhibitors in experimental models of these cardiovascular diseases significantly inhibited the progression of atherosclerotic lesion formation, neointima formation, left ventricular remodeling, pump dysfunction, and infarct healing (Creemers, et al. (2001)). In addition, MMPs have been shown to play an important role in atherosclerosis by degrading the extracellular matrix that results in cardiovascular remodeling (Ikeda and Shimada, (2003) “Matrix metalloproteinases and coronary artery diseases.” Clin Cardiol 26(2):55-9; which is herein incorporated by reference in its entirety, including for its teachings concerning the role of MMPs in cardiovascular disease and coronary artery diseases). Recent studies have shown enhanced expression of MMPs in the atherosclerotic lesion and their contribution to weakening of the vascular wall by degrading the extracellular matrix. In addition, studies have shown that polymorphism in the MMP gene promoters contribute to inter-individual differences in susceptibility to coronary heart disease (Watanabe and Ikeda, (2004) “Matrix metalloproteinases and atherosclerosis.” Curr Atheroscler Rep. 6(20:112-20; which is herein incorporated by reference in its entirety, including for its teachings concerning the role of MMPs in cardiovascular disease and atherosclerosis). Thus, according to certain embodiments the inventive fluid alleviates many of the symptoms and/or conditions of several cardiovascular diseases by modulating the levels of MMPs.

Certain embodiments herein relate to therapeutic compositions and methods of treatment for a subject by preventing or alleviating at least one symptom of cardiovascular diseases and/or an associated condition or disease.

Further embodiments herein relate to the therapeutic compositions and methods of treatment for preventing or alleviating complications related to cardiovascular diseases and/or an associated condition.

### Methods of Treatment

The term “treating” refers to, and includes, reversing, alleviating, inhibiting the progress of, or preventing a disease, disorder or condition, or one or more symptoms thereof; and “treatment” and “therapeutically” refer to the act of treating, as defined herein.

A “therapeutically effective amount” is any amount of any of the compounds utilized in the course of practicing the invention provided herein that is sufficient to reverse, alleviate, inhibit the progress of, or prevent a disease, disorder or condition, or one or more symptoms thereof.

Certain embodiments herein relate to therapeutic compositions and methods of treatment for a subject by preventing or alleviating at least one symptom of inflammation associated with certain conditions or diseases, like macular degeneration.

Many conditions or diseases associated with inflammation have been treated with steroids, methotrexate, immunosuppressive drugs including cyclophosphamide, cyclosporine, azathioprine and leflunomide, nonsteroidal anti-inflammatory agents such as aspirin, acetaminophen and COX-2 inhibitors, gold agents and anti-malarial treatments. These drugs have a variety of disadvantages, and adverse reactions including injection site reactions, rash, upper respiratory infections, autoimmune disorders and increased susceptibility to infections. In addition, many anti-inflammatory pharmaceutical drugs require intravenous (IV) or subcutaneous (SC) administration, as opposed to more convenient and compliant oral or topical dermal routes. Accordingly, a need still exists for the development of novel medicaments and treatment methods for conditions and diseases relating to inflammation.

Combination treatments. Particular embodiments comprise combination therapy using the inventive electrokinetic fluids in combination with at least one other agent, including but not limited to quinidine, procainamide, disopyramide, lidocaine, phenytoin, mexiletine, flecainide, propafenone, moricizine, propranolol, esmolol, timolol, metoprolol, atenolol, bisoprolo, amiodarone, sotalol, ibutilide, dofetilide, dronedarone, E-4031, verapamil, diltiazem, adenosine, digoxin, magnesium sulfate, warfarin, heparins, anti-platelet drugs (e.g., aspirin and clopidogrel), beta blockers (e.g., metoprolol and carvedilol), angiotensin-converting enzyme (ACE) inhibitors (e.g., captopril, zofenopril, enalapril, ramipril, quinapril, perindopril, lisinopril, benazepril, fosinopril, casokinins and lactokinins), statins (e.g., atorvastatin, cerivastatin, fluvastatin, lovastatin, pitavastatin, mevastatin, pravastatin, rosuvastatin, and simvastatin), aldosterone antagonist agents (e.g., eplerenone and spironolactone), digitalis, diuretics, digoxin, inotropes (e.g., Milrinone), vasodilators and omega-3 fatty acids.

### Anti-Inflammatory Activity of the Electrokinetically-Generated Gas-Enriched Fluids and Solutions:

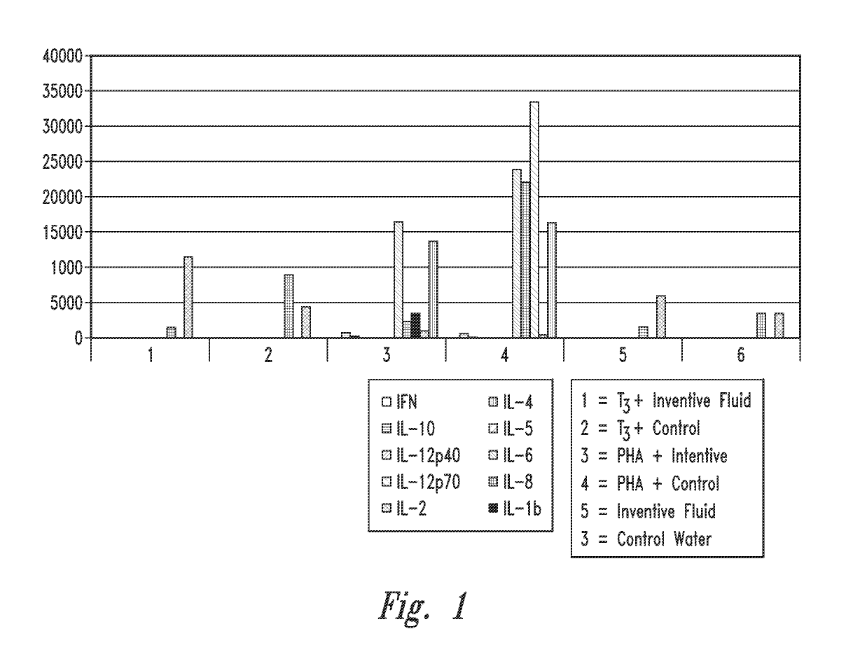

According to certain aspects of the present invention, the gas-enriched fluids and/or solutions disclosed herein have anti-inflammatory properties and effects, and can be used as anti-inflammatory agents for the treatment of subjects afflicted by diseases or disorders relating to inflammation. FIG. 1 shows the experimental results of cytokine profiles in stimulated lymphocytes from a healthy blood donor. As can be seen in FIG. 1, the inventive oxygen-enriched fluid (water) affected a down regulation of particular cytokines, especially IL-6, IL-8, and IL-1 β.

Increased production of pro-inflammatory cytokines has been implicated in the pathogenesis of numerous inflammatory and autoimmune diseases. Secretion of TNFα is a primary event in the initiation of the inflammatory cascade (Brennan F. M., et. al. Lancet, 1989, 2:244-7; Haworth C, et. al. Eur. J. Immunol. 1991, 21:2575-2579) and directly contributes to the initiation and maintenance of inflammatory and autoimmune diseases. Other pro-inflammatory cytokines also play a role, including interleukin 1β (IL-1β), IL-6, IL-8, IL-12 nitric oxide, IFN-γ and GM-CSF, while anti-inflammatory cytokines such as IL-10 may reduce disease. Cells of the immune system, macrophages in particular, secrete many of these cytokines in response to activating stimuli.

A variety of cell types are involved in the inflammatory process. Overproduction of TNFα by monocytes, macrophages and other immune cells is a key element in the pathogenesis of a multitude of diseases. Macrophages and T-cells in particular play a central role in the initiation and maintenance of the immune response. Once activated by pathological or immunogenic stimuli, macrophages respond by releasing a host of cytokines, including TNF-α, IL-1β, IL-8, IL-12, nitric oxide (NO), IL-6, GM-CSF, G-CSF, M-CSF and others. T-cells release IL-2, IL-4, INF-γ, and other inflammatory cytokines. These cytokines activate other immune cells and some can also act as independent cytotoxic agents. Excessive release of macrophage and T-cell derived inflammatory mediators can particularly lead to damage of normal cells and surrounding tissues.

Pro-inflammatory cytokines have been implicated in HIV-AIDS, and other viral infections including the cytomegalovirus, influenza virus and the herpes family of viruses. TNFα enhances the basal activity of the major immediate early enhancer/promoter of human cytomegalovirus and may play a role in reactivation of latent HCMV infection in premonocytic cells (Prosch S., et. al. Virology 1995, 208:197-206).

Additionally, a number of inflammatory cytokines contribute to mortality in patients suffering from sepsis or endotoxic shock. For example, TNα and IL-1β have a well-established central role in sepsis, septic shock and endotoxic shock. Increased levels of these cytokines are associated with fever, hypotension and shock (Smith J. W. et. al. J. Clin. Oncol. 1992, 10:1141-1152; Chapman P. B., et. al. J. Clin. Oncol. 1987, 5:1942-1951) together with the induction of gene expression for phospholipase A2 (Gronich J., et. al. J. Clin. Invest. 1994, 93:1224-1233) and NO synthase.

The induction of NO from smooth muscle cells mediates decreased mean arterial pressure and systemic vascular resistance during septic shock, suggesting a fundamental role for NO. Thus, therapies that target downregulatory effects on IL-6, IL-8, IL-1β, and NO could be beneficial in the treatment of inflammatory diseases or disorders, including macular degenerative.

IL-1 and TNFα play a central role in various acute as well as chronic responses in animal models. Additionally, IL-11, IFNα and IFNβ may also up-regulate inflammatory reactions. Conversely, several cytokines may be involved in down-regulation of inflammatory responses (i.e. IL-4, IL-10, IL-13, among others). As set forth in Examples 2 and 3, cells contacted with the inventive gas-enriched fluid showed an increase in IFN-γ levels with T3 antigen than in the control culture media with T3 antigen, while IL-8 was lower in the inventive gas-enriched culture media with T3 antigen than in the control culture media with T3 antigen. Additionally, IL-6, IL-8, and TNF-α levels were lower in the inventive gas-enriched media with PHA, than in the control media with PHA, while IL-1β levels were lower in the inventive gas-enriched fluid with PHA when compared with control media with PHA. In the inventive gas-enriched media alone, IFN-γ levels were higher than in control media. These results are consistent with an anti-inflammatory microenvironment.

NO is recognized as a mediator and regulator of inflammatory responses. It possesses cytotoxic properties toward pathogens, but can also have deleterious effects on the subject's own tissues. (Korhonen et al., Curr Drug Targets Inflamm Allergy 4(4): 471-9, 2005). NO reacts with soluble guanylate cyclase to form cyclic guanosine monophosphate (cGMP), which mediates many of the effects of NO. NO can also interact with molecular oxygen and superoxide anion to produce reactive oxygen species that can modify various cellular functions. These indirect effects of NO have a significant role in inflammation, where NO is produce in high amounts by inducible NO synthase (iNOS) and reactive oxygen species are synthesized by activated inflammatory cells.

NO can be produced by keratinocytes, fibroblasts, endothelial cells, and possibly others. Some of the vascular actions of NO include vasodilation, inhibiting platelet adhesion to the vascular endothelium, inhibiting leukocyte adhesion to the vascular endothelium, and scavenging superoxides. (Shah et al., Env. Health Persp. v. 106 (5): 1139-1143.)

Furthermore, inhibition of NO synthesis has been shown to delay wound contraction, alter. collagen organization, and alter neoepidermis thickness. (Amadeu and Costa, J. Cutan. Pathol. 33: 465-473, 2006.) Mast cell migration and angiogenesis in wounds is also affected by inhibition of NO. (Id.) Without being bound to any particular theory of mechanism, in certain embodiments the inventive gas-enriched fluids may be modulating localized and/or cellular NO production, or degradation, consistent with the spectrum of wound healing effects illustrated in the Examples section disclosed herein. Due to variable pathways of regulation, in certain embodiments, the inventive gas-enriched fluid may increase NO production and/or retard NO degradation, whereas in other certain embodiments, the inventive gas-enriched fluid may decrease NO production and/or hasten NO degradation.

In the case of mast cell migration, differences also occurred in early and late migration for the oxygen-enriched solution. This is consistent with what is known in the art regarding inhibition of NO synthesis (Amadeu and Costa, J. Cutan Pathol 33: 465-473, 2006).

In the first two phases of the inflammatory process, the foreign body is either destroyed, for example, if the foreign body is an organism, or the tissue around it is loosened, for example, if it is a splinter. In the healing phase, the inflammation begins to subside; individual blood vessels and vascular patterns become normal once again; and repair of the wound commences. The three main events in the repair process are (1) formation of new connective tissue by proliferating fibroblasts; (2) regeneration of epithelium; and (3) outgrowth of new capillaries.

Even before the inflammation subsides, fibroblasts begin moving into the injured area from the surrounding normal tissue, where they usually exist in a dormant state. They migrate by an amoeboid movement along strands of fibrin and distribute themselves throughout the healing area. Once fixed into position in the injured tissue, they begin to synthesize collagen and secrete this protein, which arranges itself into fibers. The fibers orient themselves with their longitudinal axes in the direction of the greatest stress. As the collagen bundles grow in firmness, the fibroblasts gradually degenerate and attach closely to the bundles, and the injured area transforms into scar tissue.

Simultaneously with scar tissue formation, the intact epidermal cells on the edge of the wound begin to proliferate and move, as one sheet, toward the center of the injured area. As the inflammation subsides, a need for a direct supply of blood arises, and angiogenesis occurs at the wound site.

Inflammation is a complex process that involves multiple cell types. For example, mast cells release mediators that trigger an early phase of vasodilation, accompanied by the separation of endothelial cells and exposure of collagen fibers in the subendothelial layer. Fibers in the intercellular gaps that form in blood vessels trap platelets and trigger the release of mediators from these cells.

In addition to platelets, the exposed collagen fibers also interact with proteins of the plasma that filter through the pores of the dilated vessel wall, including the triggering factor of the blood-clotting cascade, increased vasodilation, increased blood vessel permeability, and chemotaxis.

Additionally, the complement cascade can be activated by several stimuli: the injured blood vessels, the proteolytic enzymes released by the damaged cells, the membrane components of any participating bacteria, and antigen-antibody complexes. Some of the activated complement components act as chemotactic factors, responsible for the influx of leukocytes into the inflamed area, while others facilitate phagocytosis and participate in cell lysis.

In particular aspects, the inventive gas-enriched fluids or solutions also regulate at least one cytokine involved in at least one aspect of inflammation, the cytokine(s) including, but not limited to MAF (macrophage activating factor), MMIF (macrophage migration inhibition factor), MCF (macrophage chemotactic factor), LMIF (leukocyte migration inhibition factor), HRFs (histamine releasing factors), TF (transfer factors), interleukins (IL-1, IL-2, IL-3, IL-4, IL-5, IL-6, IL-7, IL-8, IL-9, IL-10, IL-11, IL-12, IL-13, IL-14, IL-15, etc.), TNF-α, TNF-β, interferons (IFN-α, IFN-β, IFN-γ, IFN-ζ, IFN-δ, etc.), G-CSF (granulocyte colony stimulating factor), GM-CSF (granulocyte-macrophage CSF), M-CSF (macrophage CSF), multi-CSF (IL-3), fibroblast growth factor (aFGF, bFGF), EGF (epidermal growth factor), NGF (nerve growth factor), PDGF (platelet-derived growth factor), VEGF (vascular endothelial growth factor), transforming growth factors (TGF-α, TGF-β, etc.), NAP-2 (neutrophil-activating protein 2), PF-4 (platelet factor 4), thromboglobulin, MCP-1 (monocyte chemoattractant protein 1), MCP-3, MIP-1α, MIP-1β-+ (macrophage inflammatory proteins), RANTES (regulated upon activation normal T expressed and presumably secreted chemokine), HSPs (heat shock proteins), GRPs (glucose-regulated proteins), ubiquitin, and others.

Thus, in certain embodiments, the gas-enriched fluids and/or therapeutic compositions increase production and/or secretion of anti-inflammatory molecules or cytokines and/or decrease the degradation of anti-inflammatory molecules or cytokines, thereby alleviating or preventing at least one symptom of inflammation (eg. macular degeneration). In other embodiments, the gas-enriched fluids and/or therapeutic compositions of the present invention decrease production and/or secretion of pro-inflammatory molecules or cytokines and/or increase the degradation of pro-inflammatory molecules or cytokines, thereby alleviating or preventing at least one symptom of inflammation and/or inflammatory neurodegeneration.

### Exemplary Relevant Molecular Interactions:

Conventionally, quantum properties are thought to belong to elementary particles of less than 10⁻¹⁰ meters, while the macroscopic world of our everyday life is referred to as classical, in that it behaves according to Newton's laws of motion.

Recently, molecules have been described as forming clusters that increase in size with dilution. These clusters measure several micrometers in diameter, and have been reported to increase in size non-linearly with dilution. Quantum coherent domains measuring 100 nanometers in diameter have been postulated to arise in pure water, and collective vibrations of water molecules in the coherent domain may eventually become phase locked to electromagnetic field fluctuations, providing for stable oscillations in water, providing a form of ‘memory’ in the form of excitation of long lasting coherent oscillations specific to dissolved substances in the water that change the collective structure of the water, which may in turn determine the specific coherent oscillations that develop. Where these oscillations become stabilized by magnetic field phase coupling, the water, upon dilution may still carry ‘seed’ coherent oscillations. As a cluster of molecules increases in size, its electromagnetic signature is correspondingly amplified, reinforcing the coherent oscillations carried by the water.

Despite variations in the cluster size of dissolved molecules and detailed microscopic structure of the water, a specificity of coherent oscillations may nonetheless exist. One model for considering changes in properties of water is based on considerations involved in crystallization.

A simplified protonated water cluster forming a nanoscale cage is shown in Applicants' previous patent application: WO 2009/055729. A protonated water cluster typically takes the form of H⁺(H₂0)ₙ. Some protonated water clusters occur naturally, such as in the ionosphere. Without being bound by any particular theory, and according to particular aspects, other types of water clusters or structures (clusters, nanocages, etc) are possible, including structures comprising oxygen and stabilized electrons imparted to the inventive output materials. Oxygen atoms may be caught in the resulting structures. The chemistry of the semi-bound nanocage allows the oxygen and/or stabilized electrons to remain dissolved for extended periods of time. Other atoms or molecules, such as medicinal compounds, can be caged for sustained delivery purposes. The specific chemistry of the solution material and dissolved compounds depend on the interactions of those materials.

Fluids processed by the mixing device have been shown previously via experiments to exhibit different structural characteristics that are consistent with an analysis of the fluid in the context of a cluster structure. See, for example, WO 2009/055729.

### Charge-Stabilized Nanostructures (e.g., Charge Stabilized Oxygen-Containing Nanostructures):

As described previously in Applicants' WO 2009/055729, “Double Layer Effect,” “Dwell Time,” “Rate of Infusion,” and “Bubble size Measurements,” the electrokinetic mixing device creates, in a matter of milliseconds, a unique non-linear fluid dynamic interaction of the first material and the second material with complex, dynamic turbulence providing complex mixing in contact with an effectively enormous surface area (including those of the device and of the exceptionally small gas bubbles of less that 100 nm) that provides for the novel electrokinetic effects described herein. Additionally, feature-localized electrokinetic effects (voltage/current) were demonstrated using a specially designed mixing device comprising insulated rotor and stator features.

As well-recognized in the art, charge redistributions and/or solvated electrons are known to be highly unstable in aqueous solution. According to particular aspects, Applicants' electrokinetic effects (e.g., charge redistributions, including, in particular aspects, solvated electrons) are surprisingly stabilized within the output material (e.g., saline solutions, ionic solutions). In fact, as described herein, the stability of the properties and biological activity of the inventive electrokinetic fluids (e.g., RNS-60 or Solas) can be maintained for months in a gas-tight container, indicating involvement of dissolved gas (e.g., oxygen) in helping to generate and/or maintain, and/or mediate the properties and activities of the inventive solutions. Significantly, the charge redistributions and/or solvated electrons are stably configured in the inventive electrokinetic ionic aqueous fluids in an amount sufficient to provide, upon contact with a living cell (e.g., mammalian cell) by the fluid, modulation of at least one of cellular membrane potential and cellular membrane conductivity (see, e.g., cellular patch clamp working Example 23 from WO 2009/055729 and as disclosed herein).

As described herein under “Molecular Interactions,” to account for the stability and biological compatibility of the inventive electrokinetic fluids (e.g., electrokinetic saline solutions), Applicants have proposed that interactions between the water molecules and the molecules of the substances (e.g., oxygen) dissolved in the water change the collective structure of the water and provide for nanoscale cage clusters, including nanostructures comprising oxygen and/or stabilized electrons imparted to the inventive output materials. Without being bound by mechanism, the configuration of the nanostructures in particular aspects is such that they: comprise (at least for formation and/or stability and/or biological activity) dissolved gas (e.g., oxygen); enable the electrokinetic fluids (e.g., RNS-60 or Solas saline fluids) to modulate (e.g., impart or receive) charges and/or charge effects upon contact with a cell membrane or related constituent thereof; and in particular aspects provide for stabilization (e.g., carrying, harboring, trapping) solvated electrons in a biologically-relevant form.

According to particular aspects, and as supported by the present disclosure, in ionic or saline (e.g., standard saline, NaCl) solutions, the inventive nanostructures comprise charge stabilized nanostrutures (e.g., average diameter less that 100 nm) that may comprise at least one dissolved gas molecule (e.g., oxygen) within a charge-stabilized hydration shell. According to additional aspects, the charge-stabilized hydration shell may comprise a cage or void harboring the at least one dissolved gas molecule (e.g., oxygen). According to further aspects, by virtue of the provision of suitable charge-stabilized hydration shells, the charge-stabilized nanostructure and/or charge-stabilized oxygen containing nano-structures may additionally comprise a solvated electron (e.g., stabilized solvated electron).

Without being bound by mechanism or particular theory, after the present priority date, charge-stabilized microbubbles stabilized by ions in aqueous liquid in equilibrium with ambient (atmospheric) gas have been proposed (Bunkin et al., Journal of Experimental and Theoretical Physics, 104:486-498, 2007; incorporated herein by reference in its entirety). According to particular aspects of the present invention, Applicants' novel electrokinetic fluids comprise a novel, biologically active form of charge-stabilized oxygen-containing nanostructures, and may further comprise novel arrays, clusters or associations of such structures.

According to the charge-stabilized microbubble model, the short-range molecular order of the water structure is destroyed by the presence of a gas molecule (e.g., a dissolved gas molecule initially complexed with a nonadsorptive ion provides a short-range order defect), providing for condensation of ionic droplets, wherein the defect is surrounded by first and second coordination spheres of water molecules, which are alternately filled by adsorptive ions (e.g., acquisition of a ‘screening shell of Na⁺ ions to form an electrical double layer) and nonadsorptive ions (e.g., Cl⁻ ions occupying the second coordination sphere) occupying six and 12 vacancies, respectively, in the coordination spheres. In under-saturated ionic solutions (e.g., undersaturated saline solutions), this hydrated ‘nucleus’ remains stable until the first and second spheres are filled by six adsorptive and five nonadsorptive ions, respectively, and then undergoes Coulomb explosion creating an internal void containing the gas molecule, wherein the adsorptive ions (e.g., Na⁺ ions) are adsorbed to the surface of the resulting void, while the nonadsorptive ions (or some portion thereof) diffuse into the solution (Bunkin et al., supra). In this model, the void in the nanostructure is prevented from collapsing by Coulombic repulsion between the ions (e.g., Na⁺ ions) adsorbed to its surface. The stability of the void-containing nanostrutures is postulated to be due to the selective adsorption of dissolved ions with like charges onto the void/bubble surface and diffusive equilibrium between the dissolved gas and the gas inside the bubble, where the negative (outward electrostatic pressure exerted by the resulting electrical double layer provides stable compensation for surface tension, and the gas pressure inside the bubble is balanced by the ambient pressure. According to the model, formation of such microbubbles requires an ionic component, and in certain aspects collision-mediated associations between particles may provide for formation of larger order clusters (arrays) (Id).

The charge-stabilized microbubble model suggests that the particles can be gas microbubbles, but contemplates only spontaneous formation of such structures in ionic solution in equilibrium with ambient air, is uncharacterized and silent as to whether oxygen is capable of forming such structures, and is likewise silent as to whether solvated electrons might be associated and/or stabilized by such structures.

According to particular aspects, the inventive electrokinetic fluids comprising charge-stabilized nanostructures and/or charge-stabilized oxygen-containing nanostructures are novel and fundamentally distinct from the postulated non-electrokinetic, atmospheric charge-stabilized microbubble structures according to the microbubble model. Significantly, this conclusion is unavoidable, deriving, at least in part, from the fact that control saline solutions do not have the biological properties disclosed herein, whereas Applicants' charge-stabilized nanostructures provide a novel, biologically active form of charge-stabilized oxygen-containing nanostructures.

According to particular aspects of the present invention, Applicants' novel electrokinetic device and methods provide for novel electrokinetically-altered fluids comprising significant quantities of charge-stabilized nanostructures in excess of any amount that may or may not spontaneously occur in ionic fluids in equilibrium with air, or in any non-electrokinetically generated fluids. In particular aspects, the charge-stabilized nanostructures comprise charge-stabilized oxygen-containing nanostructures. In additional aspects, the charge-stabilized nanostrutures are all, or substantially all charge-stabilized oxygen-containing nanostructures, or the charge-stabilized oxygen-containing nanostructures the major charge-stabilized gas-containing nanostructure species in the electrokinetic fluid.

According to yet further aspects, the charge-stabilized nanostructures and/or the charge-stabilized oxygen-containing nanostructures may comprise or harbor a solvated electron, and thereby provide a novel stabilized solvated electron carrier. In particular aspects, the charge-stabilized nanostructures and/or the charge-stabilized oxygen-containing nanostructures provide a novel type of electride (or inverted electride), which in contrast to conventional solute electrides having a single organically coordinated cation, rather have a plurality of cations stably arrayed about a void or a void containing an oxygen atom, wherein the arrayed sodium ions are coordinated by water hydration shells, rather than by organic molecules. According to particular aspects, a solvated electron may be accommodated by the hydration shell of water molecules, or preferably accommodated within the nanostructure void distributed over all the cations. In certain aspects, the inventive nanostructures provide a novel ‘super electride’ structure in solution by not only providing for distribution/stabilization of the solvated electron over multiple arrayed sodium cations, but also providing for association or partial association of the solvated electron with the caged oxygen molecule(s) in the void—the solvated electron distributing over an array of sodium atoms and at least one oxygen atom. According to particular aspects, therefore, ‘solvated electrons’ as presently disclosed in association with the inventive electrokinetic fluids, may not be solvated in the traditional model comprising direct hydration by water molecules. Alternatively, in limited analogy with dried electride salts, solvated electrons in the inventive electrokinetic fluids may be distributed over multiple charge-stabilized nanostructures to provide a ‘lattice glue’ to stabilize higher order arrays in aqueous solution.

In particular aspects, the inventive charge-stabilized nanostructures and/or the charge-stabilized oxygen-containing nanostructures are capable of interacting with cellular membranes or constituents thereof, or proteins, etc., to mediate biological activities. In particular aspects, the inventive charge-stabilized nanostructures and/or the charge-stabilized oxygen-containing nanostructures harboring a solvated electron are capable of interacting with cellular membranes or constituents thereof, or proteins, etc., to mediate biological activities.

In particular aspects, the inventive charge-stabilized nanostructures and/or the charge-stabilized oxygen-containing nanostructures interact with cellular membranes or constituents thereof, or proteins, etc., as a charge and/or charge effect donor (delivery) and/or as a charge and/or charge effect recipient to mediate biological activities. In particular aspects, the inventive charge-stabilized nanostructures and/or the charge-stabilized oxygen-containing nanostructures harboring a solvated electron interact with cellular membranes as a charge and/or charge effect donor and/or as a charge and/or charge effect recipient to mediate biological activities.

In particular aspects, the inventive charge-stabilized nanostructures and/or the charge-stabilized oxygen-containing nanostructures are consistent with, and account for the observed stability and biological properties of the inventive electrokinetic fluids, and further provide a novel electride (or inverted electride) that provides for stabilized solvated electrons in aqueous ionic solutions (e.g., saline solutions, NaCl, etc.).

In particular aspects, the charge-stabilized oxygen-containing nanostructures substantially comprise, take the form of, or can give rise to, charge-stabilized oxygen-containing nanobubbles. hi particular aspects, charge-stabilized oxygen-containing clusters provide for formation of relatively larger arrays of charge-stabilized oxygen-containing nanostructures, and/or charge-stabilized oxygen-containing nanobubbles or arrays thereof In particular aspects, the charge-stabilized oxygen-containing nanostructures can provide for formation of hydrophobic nanobubbles upon contact with a hydrophobic surface.

In particular aspects, the charge-stabilized oxygen-containing nanostructures substantially comprise at least one oxygen molecule. In certain aspects, the charge-stabilized oxygen-containing nanostructures substantially comprise at least 1, at least 2, at least 3, at least 4, at least 5, at least 10 at least 15, at least 20, at least 50, at least 100, or greater oxygen molecules. In particular aspects, charge-stabilized oxygen-containing nanostructures comprise or give rise to nanobubles (e.g., hydrophobid nanobubbles) of about 20 nm×1.5 nm, comprise about 12 oxygen molecules (e.g., based on the size of an oxygen molecule (approx 0.3 nm by 0.4 nm), assumption of an ideal gas and application of n=PV/RT, where P=1 atm, R=0.082□057□1.atm/mol.K; T=295K; V=pr²h=4.7×10⁻²² L, where r=10×10⁻⁹ m, h=1.5×10⁻⁹ m, and n=1.95×10⁻²² moles).

In certain aspects, the percentage of oxygen molecules present in the fluid that are in such nanostructures, or arrays thereof, having a charge-stabilized configuration in the ionic aqueous fluid is a percentage amount selected from the group consisting of greater than: 0.1%, 1%; 2%; 5%; 10%; 15%; 20%; 25%; 30%; 35%; 40%; 45%; 50%; 55%; 60%; 65%, 70%, 75%, 80%, 85%; 90%; and greater than 95%. Preferably, this percentage is greater than about 5%, greater than about 10%, greater than about 15% f, or greater than about 20%. In additional aspects, the substantial size of the charge-stabilized oxygen-containing nanostructures, or arrays thereof, having a charge-stabilized configuration in the ionic aqueous fluid is a size selected from the group consisting of less than: 100 nm; 90 nm; 80 nm; 70 nm; 60 nm; 50 rim; 40 nm; 30 nm; 20 nm; 10 nm; 5 nm; 4 rim; 3 rim; 2 nm; and 1 nm. Preferably, this size is less than about 50 nm, less than about 40 nm, less than about 30 nm, less than about 20 nm, or less than about 10 nm.

In certain aspects, the inventive electrokinetic fluids comprise solvated electrons. In further aspects, the inventive electrokinetic fluids comprises charge-stabilized nanostructures and/or charge-stabilized oxygen-containing nanostructures, and/or arrays thereof, which comprise at least one of: solvated electron(s); and unique charge distributions (polar, symmetric, asymmetric charge distribution). In certain aspects, the charge-stabilized nanostructures and/or charge-stabilized oxygen-containing nanostructures, and/or arrays thereof, have paramagnetic properties.

By contrast, relative to the inventive electrokinetic fluids, control pressure pot oxygenated fluids (non-electrokinetic fluids) and the like do not comprise such electrokinetically generated charge-stabilized biologically-active nanostructures and/or biologically-active charge-stabilized oxygen-containing nanostructures and/or arrays thereof, capable of modulation of at least one of cellular membrane potential and cellular membrane conductivity.

### Systems for Making Gas-Enriched Fluids

The system and methods as previously disclosed in Applicants' WO 2009/055729 patent application allow gas (e.g. oxygen) to be enriched stably at a high concentration with minimal passive loss. This system and methods can be effectively used to enrich a wide variety of gases at heightened percentages into a wide variety of fluids. By way of example only, deionized water at room temperature that typically has levels of about 2-3 ppm (parts per million) of dissolved oxygen can achieve levels of dissolved oxygen ranging from at least about 5 ppm, at least about 10 ppm, at least about 15 ppm, at least about 20 ppm, at least about 25 ppm, at least about 30 ppm, at least about 35 ppm, at least about 40 ppm, at least about 45 ppm, at least about 50 ppm, at least about 55 ppm, at least about 60 ppm, at least about 65 ppm, at least about 70 ppm, at least about 75 ppm, at least about 80 ppm, at least about 85 ppm, at least about 90 ppm, at least about 95 ppm, at least about 100 ppm, or any value greater or therebetween using the disclosed systems and/or methods. In accordance with a particular exemplary embodiment, oxygen-enriched water may be generated with levels of about 30-60 ppm of dissolved oxygen.

Table 3 illustrates various partial pressure measurements taken in a healing wound treated with an oxygen-enriched saline solution (Table 3) and in samples of the gas-enriched oxygen-enriched saline solution of the present invention.

TABLE 3
TISSUE OXYGEN MEASUREMENTS

| | Probe Z082BO |
|---|---|
| | In air: 171 mmHg 23° C. |
| Column | Partial Pressure (mmHg) |
| B1 | 32-36 |
| B2 | 169-200 |
| B3 | 20-180* |
| B4 | 40-60 |

*wound depth minimal, majority >150, occasional 20 s

### Routes and Forms of Administration

In particular exemplary embodiments, the gas-enriched fluid of the present invention may function as a therapeutic composition alone or in combination with another therapeutic agent such that the therapeutic composition prevents or alleviates at least one symptom of inflammation. The therapeutic compositions of the present invention include compositions that are able to be administered to a subject in need thereof In certain embodiments, the therapeutic composition formulation may also comprise at least one additional agent selected from the group consisting of: carriers, adjuvants, emulsifying agents, suspending agents, sweeteners, flavorings, perfumes, and binding agents.

As used herein, “pharmaceutically acceptable carrier” and “carrier” generally refer to a non-toxic, inert solid, semi-solid or liquid filler, diluent, encapsulating material or formulation auxiliary of any type. Some non-limiting examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. In particular aspects, such carriers and excipients may be gas-enriched fluids or solutions of the present invention.

The pharmaceutically acceptable carriers described herein, for example, vehicles, adjuvants, excipients, or diluents, are well known to those who are skilled in the art. Typically, the pharmaceutically acceptable carrier is chemically inert to the therapeutic agents and has no detrimental side effects or toxicity under the conditions of use. The pharmaceutically acceptable carriers can include polymers and polymer matrices, nanoparticles, microbubbles, and the like.

In addition to the therapeutic gas-enriched fluid of the present invention, the therapeutic composition may further comprise inert diluents such as additional non-gas-enriched water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. As is appreciated by those of ordinary skill, a novel and improved formulation of a particular therapeutic composition, a novel gas-enriched therapeutic fluid, and a novel method of delivering the novel gas-enriched therapeutic fluid may be obtained by replacing one or more inert diluents with a gas-enriched fluid of identical, similar, or different composition. For example, conventional water may be replaced or supplemented by a gas-enriched fluid produced by mixing oxygen into water or deionized water to provide gas-enriched fluid.

In certain embodiments, the inventive gas-enriched fluid may be combined with one or more therapeutic agents and/or used alone. In particular embodiments, incorporating the gas-enriched fluid may include replacing one or more solutions known in the art, such as deionized water, saline solution, and the like with one or more gas-enriched fluid, thereby providing an improved therapeutic composition for delivery to the subject.

Certain embodiments provide for therapeutic compositions comprising a gas-enriched fluid of the present invention, a pharmaceutical composition or other therapeutic agent or a pharmaceutically acceptable salt or solvate thereof, and at least one pharmaceutical carrier or diluent. These pharmaceutical compositions may be used in the prophylaxis and treatment of the foregoing diseases or conditions and in therapies as mentioned above. Preferably, the carrier must be pharmaceutically acceptable and must be compatible with, i.e. not have a deleterious effect upon, the other ingredients in the composition. The carrier may be a solid or liquid and is preferably formulated as a unit dose formulation, for example, a tablet that may contain from 0.05 to 95% by weight of the active ingredient.

Possible administration routes include oral, sublingual, buccal, parenteral (for example subcutaneous, intramuscular, intra-arterial, intraperitoneally, intracisternally, intravesically, intrathecally, or intravenous), rectal, topical including transdermal, intravaginal, intraoccular, intraotical, intranasal, inhalation, and injection or insertion of implantable devices or materials.

### Administration Routes

Most suitable means of administration for a particular subject will depend on the nature and severity of the disease or condition being treated or the nature of the therapy being used, as well as the nature of the therapeutic composition or additional therapeutic agent. In certain embodiments, oral or topical administration is preferred.

Formulations suitable for oral administration may be provided as discrete units, such as tablets, capsules, cachets, syrups, elixirs, chewing gum, “lollipop” formulations, microemulsions, solutions, suspensions, lozenges, or gel-coated ampules, each containing a predetermined amount of the active compound; as powders or granules; as solutions or suspensions in aqueous or non-aqueous liquids; or as oil-in-water or water-in-oil emulsions.

Additional formulations suitable for oral administration may be provided to include fine particle dusts or mists which may be generated by means of various types of metered dose pressurized aerosols, atomizers, nebulisers, or insufflators. In particular, powders or other compounds of therapeutic agents may be dissolved or suspended in a gas-enriched fluid of the present invention.

Formulations suitable for transmucosal methods, such as by sublingual or buccal administration include lozenges patches, tablets, and the like comprising the active compound and, typically a flavored base, such as sugar and acacia or tragacanth and pastilles comprising the active compound in an inert base, such as gelatin and glycerine or sucrose acacia.

Formulations suitable for parenteral administration typically comprise sterile aqueous solutions containing a predetermined concentration of the active gas-enriched fluid and possibly another therapeutic agent; the solution is preferably isotonic with the blood of the intended recipient. Additional formulations suitable for parenteral administration include formulations containing physiologically suitable co-solvents and/or complexing agents such as surfactants and cyclodextrins. Oil-in-water emulsions may also be suitable for formulations for parenteral administration of the gas-enriched fluid. Although such solutions are preferably administered intravenously, they may also be administered by subcutaneous or intramuscular injection.

Formulations suitable for urethral, rectal or vaginal administration include gels, creams, lotions, aqueous or oily suspensions, dispersible powders or granules, emulsions, dissolvable solid materials, douches, and the like. The formulations are preferably provided as unit-dose suppositories comprising the active ingredient in one or more solid carriers forming the suppository base, for example, cocoa butter. Alternatively, colonic washes with the gas-enriched fluids of the present invention may be formulated for colonic or rectal administration.

Formulations suitable for topical, intraoccular, intraotic, or intranasal application include ointments, creams, pastes, lotions, pastes, gels (such as hydrogels), sprays, dispersible powders and granules, emulsions, sprays or aerosols using flowing propellants (such as liposomal sprays, nasal drops, nasal sprays, and the like) and oils. Suitable carriers for such formulations include petroleum jelly, lanolin, polyethyleneglycols, alcohols, and combinations thereof. Nasal or intranasal delivery may include metered doses of any of these formulations or others. Likewise, intraotic or intraocular may include drops, ointments, irritation fluids and the like.

Formulations of the invention may be prepared by any suitable method, typically by uniformly and intimately admixing the gas-enriched fluid optionally with an active compound with liquids or finely divided solid carriers or both, in the required proportions and then, if necessary, shaping the resulting mixture into the desired shape.

For example a tablet may be prepared by compressing an intimate mixture comprising a powder or granules of the active ingredient and one or more optional ingredients, such as a binder, lubricant, inert diluent, or surface active dispersing agent, or by molding an intimate mixture of powdered active ingredient and a gas-enriched fluid of the present invention.

Suitable formulations for administration by inhalation include fine particle dusts or mists which may be generated by means of various types of metered dose pressurized aerosols, atomizers, nebulisers, or insufflators. In particular, powders or other compounds of therapeutic agents may be dissolved or suspended in a gas-enriched fluid of the present invention. For pulmonary administration via the mouth, the particle size of the powder or droplets is typically in the range 0.5-10 μM, preferably 1-5 μM, to ensure delivery into the bronchial tree. For nasal administration, a particle size in the range 10-500 μM is preferred to ensure retention in the nasal cavity.

Metered dose inhalers are pressurized aerosol dispensers, typically containing a suspension or solution formulation of a therapeutic agent in a liquefied propellant. In certain embodiments, as disclosed herein, the gas-enriched fluids of the present invention may be used in addition to or instead of the standard liquefied propellant. During use, these devices discharge the formulation through a valve adapted to deliver a metered volume, typically from 10 to 150 μL, to produce a fine particle spray containing the therapeutic agent and the gas-enriched fluid. Suitable propellants include certain chlorofluorocarbon compounds, for example, dichlorodifluoromethane, trichlorofluoromethane, dichlorotetrafluoroethane and mixtures thereof.

The formulation may additionally contain one or more co-solvents, for example, ethanol surfactants, such as oleic acid or sorbitan trioleate, anti-oxidants and suitable flavoring agents. Nebulisers are commercially available devices that transform solutions or suspensions of the active ingredient into a therapeutic aerosol mist either by means of acceleration of a compressed gas (typically air or oxygen) through a narrow venturi orifice, or by means of ultrasonic agitation. Suitable formulations for use in nebulisers consist of another therapeutic agent in a gas-enriched fluid and comprising up to 40% w/w of the formulation, preferably less than 20% w/w. In addition, other carriers may be utilized, such as distilled water, sterile water, or a dilute aqueous alcohol solution, preferably made isotonic with body fluids by the addition of salts, such as sodium chloride. Optional additives include preservatives, especially if the formulation is not prepared sterile, and may include methyl hydroxy-benzoate, anti-oxidants, flavoring agents, volatile oils, buffering agents and surfactants.

Suitable formulations for administration by insufflation include finely comminuted powders that may be delivered by means of an insufflator or taken into the nasal cavity in the manner of a snuff In the insufflator, the powder is contained in capsules or cartridges, typically made of gelatin or plastic, which are either pierced or opened in situ and the powder delivered by air drawn through the device upon inhalation or by means of a manually-operated pump. The powder employed in the insufflator consists either solely of the active ingredient or of a powder blend comprising the active ingredient, a suitable powder diluent, such as lactose, and an optional surfactant. The active ingredient typically comprises from 0.1 to 100 w/w of the formulation.

In addition to the ingredients specifically mentioned above, the formulations of the present invention may include other agents known to those skilled in the art, having regard for the type of formulation in issue. For example, formulations suitable for oral administration may include flavoring agents and formulations suitable for intranasal administration may include perfumes.

The therapeutic compositions of the invention can be administered by any conventional method available for use in conjunction with pharmaceutical drugs, either as individual therapeutic agents or in a combination of therapeutic agents.

The dosage administered will, of course, vary depending upon known factors, such as the pharmacodynamic characteristics of the particular agent and its mode and route of administration; the age, health and weight of the recipient; the nature and extent of the symptoms; the kind of concurrent treatment; the frequency of treatment; and the effect desired. A daily dosage of active ingredient can be expected to be about 0.001 to 1000 milligrams (mg) per kilogram (kg) of body weight, with the preferred dose being 0.1 to about 30 mg/kg. According to certain aspects daily dosage of active ingredient may be 0.001 liters to 10 liters, with the preferred dose being from about 0.01 liters to 1 liter.

Dosage forms (compositions suitable for administration) contain from about 1 mg to about 500 mg of active ingredient per unit. In these pharmaceutical compositions, the active ingredient will ordinarily be present in an amount of about 0.5-95% weight based on the total weight of the composition.

Ointments, pastes, foams, occlusions, creams and gels also can contain excipients, such as starch, tragacanth, cellulose derivatives, silicones, bentonites, silica acid, and talc, or mixtures thereof Powders and sprays also can contain excipients such as lactose, talc, silica acid, aluminum hydroxide, and calcium silicates, or mixtures of these substances. Solutions of nanocrystalline antimicrobial metals can be converted into aerosols or sprays by any of the known means routinely used for making aerosol pharmaceuticals. In general, such methods comprise pressurizing or providing a means for pressurizing a container of the solution, usually with an inert carrier gas, and passing the pressurized gas through a small orifice. Sprays can additionally contain customary propellants, such as nitrogen, carbon dioxide, and other inert gases. In addition, microspheres or nanoparticles may be employed with the gas-enriched therapeutic compositions or fluids of the present invention in any of the routes required to administer the therapeutic compounds to a subject.

The injection-use formulations can be presented in unit-dose or multi-dose sealed containers, such as ampules and vials, and can be stored in a freeze-dried (lyophilized) condition requiring only the addition of the sterile liquid excipient, or gas-enriched fluid, immediately prior to use. Extemporaneous injection solutions and suspensions can be prepared from sterile powders, granules, and tablets. The requirements for effective pharmaceutical carriers for injectable compositions are well known to those of ordinary skill in the art. See, for example, Pharmaceutics and Pharmacy Practice, J. B. Lippincott Co., Philadelphia, Pa., Banker and Chalmers, Eds., 238-250 (1982) and ASHP Handbook on Injectable Drugs, Toissel, 4th ed., 622-630 (1986).

Formulations suitable for topical administration include lozenges comprising a gas-enriched fluid of the invention and optionally, an additional therapeutic and a flavor, usually sucrose and acacia or tragacanth; pastilles comprising a gas-enriched fluid and optional additional therapeutic agent in an inert base, such as gelatin and glycerin, or sucrose and acacia; and mouth washes or oral rinses comprising a gas-enriched fluid and optional additional therapeutic agent in a suitable liquid carrier; as well as creams, emulsions, gels and the like.

Additionally, formulations suitable for rectal administration may be presented as suppositories by mixing with a variety of bases such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

Suitable pharmaceutical carriers are described in Remington's Pharmaceutical Sciences, Mack Publishing Company, a standard reference text in this field.

The dose administered to a subject, especially an animal, particularly a human, in the context of the present invention should be sufficient to affect a therapeutic response in the animal over a reasonable time frame. One skilled in the art will recognize that dosage will depend upon a variety of factors including the condition of the animal, the body weight of the animal, as well as the condition being treated. A suitable dose is that which will result in a concentration of the therapeutic composition in a subject that is known to affect the desired response.

The size of the dose also will be determined by the route, timing and frequency of administration as well as the existence, nature, and extent of any adverse side effects that might accompany the administration of the therapeutic composition and the desired physiological effect.

It will be appreciated that the compounds of the combination may be administered: (1) simultaneously by combination of the compounds in a co-formulation or (2) by alternation, i.e. delivering the compounds serially, sequentially, in parallel or simultaneously in separate pharmaceutical formulations. In alternation therapy, the delay in administering the second, and optionally a third active ingredient, should not be such as to lose the benefit of a synergistic therapeutic effect of the combination of the active ingredients. According to certain embodiments by either method of administration (1) or (2), ideally the combination should be administered to achieve the most efficacious results. In certain embodiments by either method of administration (1) or (2), ideally the combination should be administered to achieve peak plasma concentrations of each of the active ingredients. A one pill once-per-day regimen by administration of a combination co-formulation may be feasible for some patients suffering from macular degeneration. According to certain embodiments effective peak plasma concentrations of the active ingredients of the combination will be in the range of approximately 0.001 to 100 μM. Optimal peak plasma concentrations may be achieved by a formulation and dosing regimen prescribed for a particular patient. It will also be understood that the inventive fluids and at least one additional therapeutic agent is selected from the group consisting of anti-angiogenesis (anti-VEGF) therapy, simple dietary supplements, and statins or the physiologically functional derivatives of any thereof, whether presented simultaneously or sequentially, may be administered individually, in multiples, or in any combination thereof. In general, during alternation therapy (2), an effective dosage of each compound is administered serially, where in co-formulation therapy (1), effective dosages of two or more compounds are administered together.

The combinations of the invention may conveniently be presented as a pharmaceutical formulation in a unitary dosage form. A convenient unitary dosage formulation contains the active ingredients in any amount from 1 mg to 1 g each, for example but not limited to, 10 mg to 300 mg. The synergistic effects of the inventive fluid in combination with quinidine, procainamide, disopyramide, lidocaine, phenytoin, mexiletine, flecainide, propafenone, moricizine, propranolol, esmolol, timolol, metoprolol, atenolol, bisoprolo, amiodarone, sotalol, ibutilide, dofetilide, dronedarone, E-4031, verapamil, diltiazem, adenosine, digoxin, magnesium sulfate, warfarin, heparins, anti-platelet drugs (e.g., aspirin and clopidogrel), beta blockers (e.g., metoprolol and carvedilol), angiotensin-converting enzyme (ACE) inhibitors (e.g., captopril, zofenopril, enalapril, ramipril, quinapril, perindopril, lisinopril, benazepril, fosinopril, casokinins and lactokinins), statins (e.g., atorvastatin, cerivastatin, fluvastatin, lovastatin, pitavastatin, mevastatin, pravastatin, rosuvastatin, and simvastatin), aldosterone antagonist agents (e.g., eplerenone and spironolactone), digitalis, diuretics, digoxin, inotropes (e.g., Milrinone), vasodilators and omega-3 fatty acids (combination therapy)) may be realized over a wide ratio, for example 1:50 to 50:1 (inventive fluid:additional treatment). In one embodiment the ratio may range from about 1:10 to 10:1. In another embodiment, the weight/weight ratio of inventive fluid to quinidine, procainamide, disopyramide, lidocaine, phenytoin, mexiletine, flecainide, propafenone, moricizine, propranolol, esmolol, timolol, metoprolol, atenolol, bisoprolo, amiodarone, sotalol, ibutilide, dofetilide, dronedarone, E-4031, verapamil, diltiazem, adenosine, digoxin, magnesium sulfate, warfarin, heparins, anti-platelet drugs (e.g., aspirin and clopidogrel), beta blockers (e.g., metoprolol and carvedilol), angiotensin-converting enzyme (ACE) inhibitors (e.g., captopril, zofenopril, enalapril, ramipril, quinapril, perindopril, lisinopril, benazepril, fosinopril, casokinins and lactokinins), statins (e.g., atorvastatin, cerivastatin, fluvastatin, lovastatin, pitavastatin, mevastatin, pravastatin, rosuvastatin, and simvastatin), aldosterone antagonist agents (e.g., eplerenone and spironolactone), digitalis, diuretics, digoxin, inotropes (e.g., Milrinone), vasodilators and omega-3 fatty acids (combination therapy)) in a co-formulated combination dosage form, such as a pill, tablet, caplet or capsule will be about 1, i.e. an approximately equal amount of inventive fluid and quinidine, procainamide, disopyramide, lidocaine, phenytoin, mexiletine, flecainide, propafenone, moricizine, propranolol, esmolol, timolol, metoprolol, atenolol, bisoprolo, amiodarone, sotalol, ibutilide, dofetilide, dronedarone, E-4031, verapamil, diltiazem, adenosine, digoxin, magnesium sulfate, warfarin, heparins, anti-platelet drugs (e.g., aspirin and clopidogrel), beta blockers (e.g., metoprolol and carvedilol), angiotensin-converting enzyme (ACE) inhibitors (e.g., captopril, zofenopril, enalapril, ramipril, quinapril, perindopril, lisinopril, benazepril, fosinopril, casokinins and lactokinins), statins (e.g., atorvastatin, cerivastatin, fluvastatin, lovastatin, pitavastatin, mevastatin, pravastatin, rosuvastatin, and simvastatin), aldosterone antagonist agents (e.g., eplerenone and spironolactone), digitalis, diuretics, digoxin, inotropes (e.g., Milrinone), vasodilators and omega-3 fatty acids. In other exemplary co-formulations, there may be more or less inventive fluid and quinidine, procainamide, disopyramide, lidocaine, phenytoin, mexiletine, flecainide, propafenone, moricizine, propranolol, esmolol, timolol, metoprolol, atenolol, bisoprolo, amiodarone, sotalol, ibutilide, dofetilide, dronedarone, E-4031, verapamil, diltiazem, adenosine, digoxin, magnesium sulfate, warfarin, heparins, anti-platelet drugs (e.g., aspirin and clopidogrel), beta blockers (e.g., metoprolol and carvedilol), angiotensin-converting enzyme (ACE) inhibitors (e.g., captopril, zofenopril, enalapril, ramipril, quinapril, perindopril, lisinopril, benazepril, fosinopril, casokinins and lactokinins), statins (e.g., atorvastatin, cerivastatin, fluvastatin, lovastatin, pitavastatin, mevastatin, pravastatin, rosuvastatin, and simvastatin), aldosterone antagonist agents (e.g., eplerenone and spironolactone), digitalis, diuretics, digoxin, inotropes (e.g., Milrinone), vasodilators and omega-3 fatty acids. In one embodiment, each compound will be employed in the combination in an amount at which it exhibits anti-inflammatory activity when used alone. Other ratios and amounts of the compounds of said combinations are contemplated within the scope of the invention.

A unitary dosage form may further comprise inventive fluid and quinidine, procainamide, disopyramide, lidocaine, phenytoin, mexiletine, flecainide, propafenone, moricizine, propranolol, esmolol, timolol, metoprolol, atenolol, bisoprolo, amiodarone, sotalol, ibutilide, dofetilide, dronedarone, E-4031, verapamil, diltiazem, adenosine, digoxin, magnesium sulfate, warfarin, heparins, anti-platelet drugs (e.g., aspirin and clopidogrel), beta blockers (e.g., metoprolol and carvedilol), angiotensin-converting enzyme (ACE) inhibitors (e.g., captopril, zofenopril, enalapril, ramipril, quinapril, perindopril, lisinopril, benazepril, fosinopril, casokinins and lactokinins), statins (e.g., atorvastatin, cerivastatin, fluvastatin, lovastatin, pitavastatin, mevastatin, pravastatin, rosuvastatin, and simvastatin), aldosterone antagonist agents (e.g., eplerenone and spironolactone), digitalis, diuretics, digoxin, inotropes (e.g., Milrinone), vasodilators and omega-3 fatty acids, or physiologically functional derivatives of either thereof, and a pharmaceutically acceptable carrier.

It will be appreciated by those skilled in the art that the amount of active ingredients in the combinations of the invention required for use in treatment will vary according to a variety of factors, including the nature of the condition being treated and the age and condition of the patient, and will ultimately be at the discretion of the attending physician or health care practitioner. The factors to be considered include the route of administration and nature of the formulation, the animal's body weight, age and general condition and the nature and severity of the disease to be treated.

It is also possible to combine any two of the active ingredients in a unitary dosage form for simultaneous or sequential administration with a third active ingredient. The three-part combination may be administered simultaneously or sequentially. When administered sequentially, the combination may be administered in two or three administrations. According to certain embodiments the three-part combination of inventive fluid and quinidine, procainamide, disopyramide, lidocaine, phenytoin, mexiletine, flecainide, propafenone, moricizine, propranolol, esmolol, timolol, metoprolol, atenolol, bisoprolo, amiodarone, sotalol, ibutilide, dofetilide, dronedarone, E-4031, verapamil, diltiazem, adenosine, digoxin, magnesium sulfate, warfarin, heparins, anti-platelet drugs (e.g., aspirin and clopidogrel), beta blockers (e.g., metoprolol and carvedilol), angiotensin-converting enzyme (ACE) inhibitors (e.g., captopril, zofenopril, enalapril, ramipril, quinapril, perindopril, lisinopril, benazepril, fosinopril, casokinins and lactokinins), statins (e.g., atorvastatin, cerivastatin, fluvastatin, lovastatin, pitavastatin, mevastatin, pravastatin, rosuvastatin, and simvastatin), aldosterone antagonist agents (e.g., eplerenone and spironolactone), digitalis, diuretics, digoxin, inotropes (e.g., Milrinone), vasodilators and omega-3 fatty acids.

The following examples are meant to be illustrative only and not limiting in any way.

### EXAMPLE 1

### Microbubble Size

This Example is based on Example 2 of Applicants' published U.S. patent application Ser. No. 11/978,137, incorporated herein by reference for its teachings regarding bubble size. Experiments were performed with a gas-enriched fluid by using the diffuser of the present invention in order to determine a gas microbubble size limit. The microbubble size limit was established by passing the gas enriched fluid through 0.22 and 0.1 micron filters. In performing these tests, a volume of fluid passed through the diffuser of the present invention and generated a gas-enriched fluid. Sixty milliliters of this fluid was drained into a 60 ml syringe. The dissolved oxygen level of the fluid within the syringe was then measured by Winkler titration. The fluid within the syringe was injected through a 0.22 micron Millipore Millex GP50 filter and into a 50 ml beaker. The dissolved oxygen rate of the material in the 50 ml beaker was then measured. The experiment was performed three times to achieve the results illustrated in Table 4 below.

TABLE 4
Dissolve oxygen measurements after filtration.

| | DO AFTER 0.22 MICRON |
|---|---|
| DO IN SYRINGE | FILTER |
| 42.1 ppm | 39.7 ppm |
| 43.4 ppm | 42.0 ppm |
| 43.5 ppm | 39.5 ppm |

As can be seen, the dissolved oxygen levels that were measured within the syringe and the dissolved oxygen levels within the 50 ml beaker were not significantly changed by passing the diffused material through a 0.22 micron filter, which implies that the microbubbles of dissolved gas within the fluid are not larger than 0.22 microns.

A second test was performed in which a batch of saline solution was enriched with the diffuser of the present invention and a sample of the output solution was collected in an unfiltered state. The dissolved oxygen level of the unfiltered sample was 44.7 ppm. A 0.1 micron filter was used to filter the oxygen-enriched solution from the diffuser of the present invention and two additional samples were taken. For the first sample, the dissolved oxygen level was 43.4 ppm. For the second sample, the dissolved oxygen level was 41.4 ppm. Finally, the filter was removed and a final sample was taken from the unfiltered solution. In this case, the final sample had a dissolved oxygen level of 45.4 ppm. These results were consistent with those in which the Millipore 0.22 micron filter was used.

Thus, the majority of the gas bubbles or microbubbles within the saline solution are approximately less than 0.1 microns in size. This result has been cooroborated by both AFM measurements, and by laser spectroscopy studies.

### EXAMPLE 2

### A Cytokine Profile was Determined

This Example is based on Example 5 of Applicants' published U.S. patent application Ser. No. 12/435,356, incorporated herein by reference for its teachings regarding cytokine profile responses to electrokinetic fluids. Mixed lymphocytes were obtained from a single healthy volunteer donor. Buffy coat samples were washed according to standard procedures to remove platelets. Lymphocytes were plated at a concentration of 2×10⁶ per plate in RPMI media (+50 mm HEPES) diluted with either inventive gas-enriched fluid or distilled water (control). Cells were stimulated with 1 microgram/mL T3 antigen, or 1 microgram/mL phytohemagglutinin (PHA) lectin (pan-T cell activator), or unstimulated (negative control). Following 24-hour incubation, cells were checked for viability and the supernatants were extracted and frozen.

The supernatants were thawed, centrifuged, and tested for cytokine expression using a XMAP® (Luminex) bead lite protocol and platform.

Two million cells were plated into 6 wells of a 24-well plate in full RPMI+50 mm Hepes with either inventive oxygen-enriched fluid (water) (wells 1, 3,and 5) or distilled water (2, 4 and 6) (10× RPMI diluted into water to make 1×). Cells were stimulated with 1 ug/ml T3 antigen (wells 1 and 2) or PHA (wells 3 and 4). Control wells 5 and 6 were not stimulated. After 24 hours, cells were checked for viability and supernatants were collected and frozen. Next, the supernatants were thawed and spun at 8,000 g to pellet. The clarified supernatants were assayed for the cytokines listed using a LUMINEX BEAD LITE™ protocol and platform. The numerical data is tabulated in Table 5, and the corresponding bar graphs are depicted in FIG. 1. Notably, IFN-γ level was higher in the inventive gas-enriched culture media with T3 antigen than in the control culture media with T3 antigen, while IL-8 was lower in the inventive gas-enriched culture media with T3 antigen than in the control culture media with T3 antigen. Additionally, IL-6, IL-8, and TNF-α levels were lower in the inventive gas-enriched media with PHA, than in the control media with PHA, while IL-1β levels were lower in the inventive gas-enriched fluid with PHA when compared with control media with PHA. In the inventive gas-enriched media alone, IFN-y levels were higher than in control media.

TABLE 5

| Sample | IFN | Il-10 | Il-12p40 | Il-12p70 | Il-2 | Il-4 | Il-5 | Il-6 | Il-8 | Il-ib | IP-10 | TNFa |
|---|---|---|---|---|---|---|---|---|---|---|---|---|
| 1 | 0 | 0 | 0 | 2.85 | 0 | 0 | 7.98 | 20.3 | 1350 | 7.56 | 11500 | 15.5 |
| 2 | 0 | 0 | 0 | 3.08 | 0 | 0 | 8 | 15.2 | 8940 | 3.68 | 4280 | 7.94 |
| 3 | 0 | 581 | 168 | 3.15 | 0 | 0 | 8 | 16400 | 2200 | 3280 | 862 | 13700 |
| 4 | 0 | 377 | 56.3 | 4.22 | 0 | 0 | 8.08 | 23800 | 22100 | 33600 | 558 | 16200 |
| 5 | 0 | 0 | 0 | 2.51 | 0 | 0 | 7.99 | 24 | 1330 | 7.33 | 5900 | 8.55 |
| 6 | 0 | 0 | 0 | 2.77 | 0 | 0 | 8 | 5.98 | 3210 | 4.68 | 3330 | 0 |

### EXAMPLE 3

### Cytokine Expression

This Example is based on Example 6 of Applicants' published U.S. patent application Ser. No. 12/435,356, incorporated herein by reference for its teachings regarding cytokine profile responses to electrokinetic fluids. In particular aspects, human mixed lymphocytes were stimulated with T3 antigen or PHA in inventive electrokinetic fluid, or control fluid, and changes in IL-1β, IL-2, IL-4, IL-5, IL-6, IL-7, IL-8, IL-10, IL-12(p40), IL-12(p70), IL-13, IL-17, Eotaxin, IFN-γ, GM-CSF, MIP-1β, MCP-1, G-CSF, FGFb, VEGF, TNF-α, RANTES, Leptin, TNF-β, TFG-β, and NGF were evaluated. As can be seen from FIG. 1, pro-inflammatory cytokines (IL-1β, TNF-α, IL-6, and GM-CSF), chemokines (IL-8, MIP-1α, RANTES, and Eotaxin), inflammatory enzymes (iNOS, COX-2, and MMP-9), allergen responses (MHC class II, CD23, B7-1, and B7-2), and Th2 cytokines (IL-4, IL-13, and IL-5) tested were reduced in test fluid versus control fluid. By contrast, anti-inflammatory cytokines (e.g., IL1R-α, TIMPs) tested were increased in test fluid versus control fluid.

To expand on these data, Applicants used an art recognized model system involving ovalbumin sensitization, for assessing allergic hypersensitivity reactions. The end points studied were particular cytologic and cellular components of the reaction as well as serologic measurements of protein and LDH. Cytokine analysis was performed, including analysis of Eotaxin, IL-1α, IL-1β, KC, MCP-1, MCP-3, MIP-1α, RANTES, TNF-α, and VCAM.

Briefly, male Brown Norway rats were injected intraperitoneally with 0.5 mL Ovalbumin (OVA) Grade V (A5503-1G, Sigma) in solution (2.0 mg/mL) containing aluminum hydroxide (Al(OH)₃) (200 mg/mL) once each on days 1, 2, and 3. The study was a randomized 2×2 factorial arrangement of treatments (4 groups). After a two week waiting period to allow for an immune reaction to occur, the rats were either exposed or were treated for a week with either RDC1676-00 (sterile saline processed through the Revalesio proprietary device), and RDC1676-01 (sterile saline processed through the Revalesio proprietary device with additional oxygen added). At the end of the 1 week of treatment for once a day, the 2 groups were broken in half and 50% of the rats in each group received either Saline or OVA challenge by inhalation.

Specifically, fourteen days following the initial serialization, 12 rats were exposed to RDC 1676-00 by inhalation for 30 minutes each day for 7 consecutive days. The air flow rate through the system was set at 10 liters/minute. A total of 12 rats were aligned in the pie chamber, with a single port for nebulized material to enter and evenly distribute to the 12 sub-chambers of the Aeroneb.

Fifteen days following initial sensitization, 12 rats were exposed to RDC 1676-01 by ultrasonic nebulization for 30 minutes each day for 7 consecutive days. The air flow was also set for 10 liters/minute, using the same nebulizer and chamber. The RDC 1676-00 was nebulized first and the Aeroneb chamber thoroughly dried before RDC 1676-01 was nebulized.

Approximately 2 hours after the last nebulization treatment, 6 rats from the RDC 1676-00 group were re-challenged with OVA (1% in saline) delivered by intratreacheal instillation using a Penn Century Microsprayer (Model 1A-1B). The other 6 rats from the RDC 1676-00 group were challenged with saline as the control group delivered by way of intratreacheal instillation. The following day, the procedure was repeated with the RDC 1676-01 group.

Twenty four hours after re-challenge, all rats in each group were euthanized by overdose with sodium pentobarbital. Whole blood samples were collected from the inferior vena-cava and placed into two disparate blood collection tubes: Qiagen PAXgene™ Blood RNA Tube and Qiagen PAXgene™ Blood DNA Tube. Lung organs were processed to obtain bronchoalveolar lavage (BAL) fluid and lung tissue for RT-PCR to assess changes in markers of cytokine expression known to be associated with lung inflammation in this model. A unilateral lavage technique was be employed in order to preserve the integrity of the 4 lobes on the right side of the lung. The left “large”lobe was lavaged, while the 4 right lobes were tied off and immediately placedinot TRI-zol™, homogenized, and sent to the lab for further processing.

BAL analysis. Lung lavage was collected and centrifuged for 10 minutes at 4° C. at 600-800 g to pellet the cells. The supernatants were transferred to fresh tubes and frozen at −80° C. Bronchial lavage fluid (“BAL”) was separated into two aliquots. The first aliquot was spun down, and the supernatant was snap frozen on crushed dry ice, placed in −80° C., and shipped to the laboratory for further processing. The amount of protein and LDH present indicates the level of blood serum protein (the protein is a serum component that leaks through the membranes when it's challenged as in this experiment) and cell death, respectively. The proprietary test side showed slight less protein than the control.

The second aliquot of bronchial lavage fluid was evaluated for total protein and LDH content, as well as subjected to cytological examination. The treated group showed total cells to be greater than the saline control group. Further, there was an increase in eosinophils in the treated group versus the control group. There were also slightly different polymorphonuclear cells for the treated versus the control side.

Blood analysis. Whole blood was analyzed by transfer of 1.2-2.0 mL blood into a tube, and allowing it to clot for at least 30 minutes. The remaining blood sample (approximately 3.5-5.0 mL) was saved for RNA extraction using TRI-zol™ or PAXgene™. Next, the clotted blood sample was centrifuged for 10 minutes at 1200 g at room temperature. The serum (supernatant) was removed and placed into two fresh tubes, and the serum was stored at −80° C.

For RNA extraction utilizing Tri-Reagent (TB-126, Molecular Research Center, Inc.), 0.2 mL of whole blood or plasma was added to 0.75 mL of TRI Reagent BD supplemented with 20 μL of 5N acetic acid per 0.2 mL of whole blood or plasma. Tubes were shaken and stored at −80° C. Utilizing PAXgene™, tubes were incubated for approximately two hours at room temperature. Tubes were then placed on their side and stored in the −20° C. freezer for 24 hours, and then transferred to −80° C. for long term storage.

Luminex analysis. By Luminex platform, a microbead analysis was utilized as a substrate for an antibody-related binding reaction which is read out in luminosity units and can be compared with quantified standards. Each blood sample was run as 2 samples concurrently. The units of measurement are luminosity units and the groups are divided up into OVA challenged controls, OVA challenged treatment, and saline challenged treatment with proprietary fluid.

For Agilant gene array data generation, lung tissue was isolated and submerged in TRI Reagent (TR118, Molecular Research Center, Inc.). Briefly, approximately 1 mL of TRI Reagent was added to 50-100 mg of tissue in each tube. The samples were homogenized in TRI Reagent, using glass-Teflon™ or Polytron™ homogenizer. Samples were stored at −80° C.

### Blood Samples:

FIGS. 2-11 show the results of whole blood sample evaluations.

Exemplary FIG. 2 shows the basic luminosity data presentation format for the blood sample data. Letters designating the identity of the measured cytokine (in this case KC) are at the top right of each data figure. The data is presented both as data points (upper graph) and bar graphs (lower graph) of the individual samples. In either case, the graphs are divided, from left to right, in four groups. The first 2 groups (RDC1676-00 OVA and RDC1676-01 OVA, respectively) were those that were re-challenged with OVA by inhalation, whereas the last two groups (RDC1676-00 OVA and RDC1676-01 OVA, respectively) where those that were re-challenged with saline control only. Again, the suffix 00 represents saline treatment and suffix 01 represents inventive electrokinetic fluid treated groups.

Each blood sample was split into 2 samples and the samples were run concurrently. The units of measure are units of luminosity and the groups, going from left to right are: OVA challenged controls; OVA challenged inventive electrokinetic fluid treatment; followed by saline challenged saline treatment; and saline challenged inventive electrokinetic fluid treatment. To facilitate review, both the RDC1676-01 groups are highlighted with gray shaded backdrops, whereas the control saline treatment groups have unshaded backdrops.

Generally, in comparing the two left groups, while the spread of the RDC1676-01 group data is somewhat greater, particular cytokine levels in the RDC1676-01 group as a whole are less than the samples in the control treated group; typically about a 30% numerical difference between the 2 groups. Generally, in comparing the right-most two groups, the RDC1676-01 group has a slightly higher numerical number compared to the RDC1676-00 group.

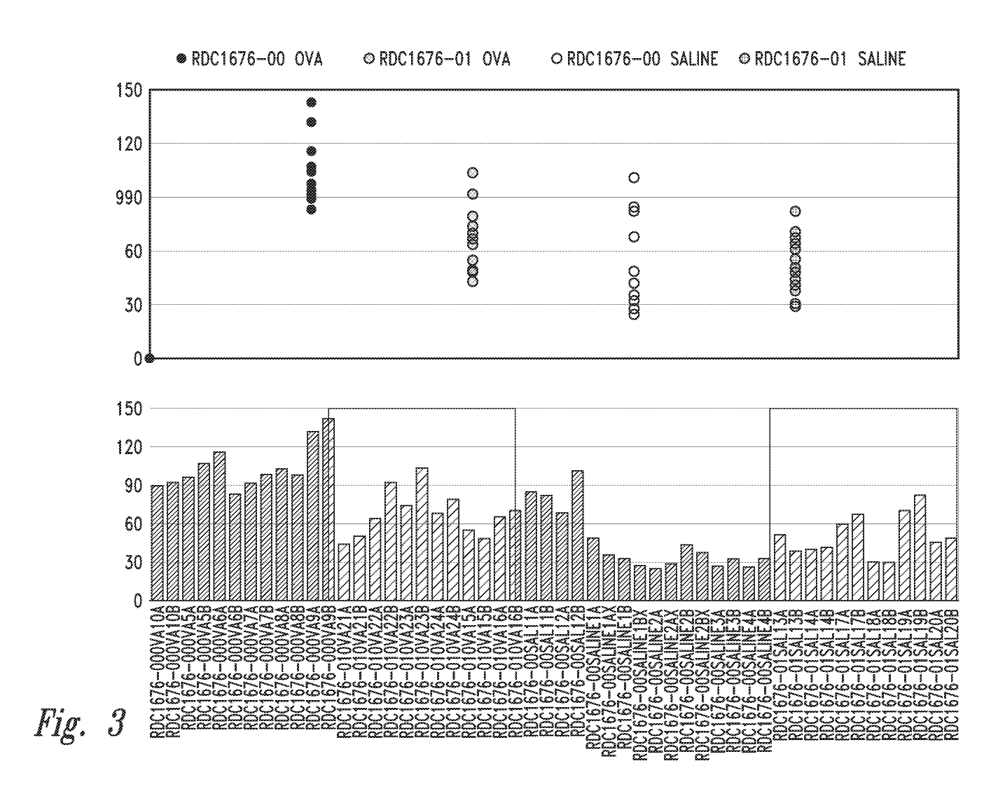

FIG. 3 shows analysis of RANTES (IL-8 super family) in blood sample data according to particular exemplary aspects. Luminosity units for the leftmost two groups (the OVA challenged groups) indicate that generally values in the RDC1676-01 treated group were less than the RDC 1676-00 control group as shown by the dot plot in the upper graph portion which again shows a 30-35% differential between the two groups, whereas in the saline only exposed groups the cytokine level values where roughly the same, or perhaps slightly increased in the RDC1676-01 treated group.

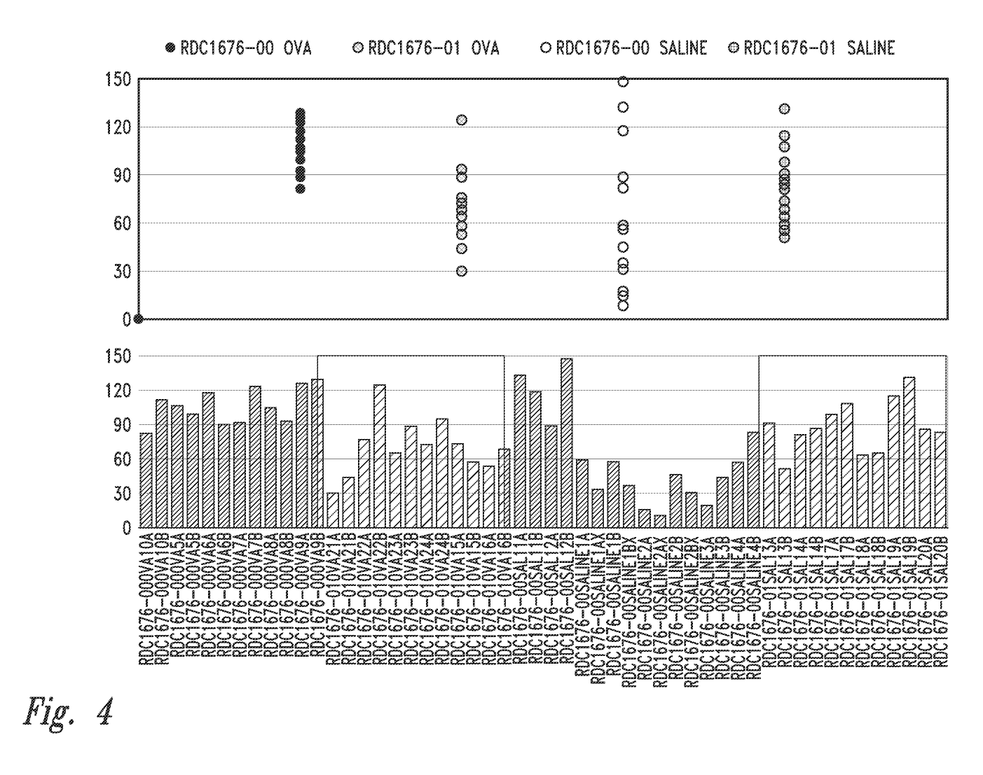

FIG. 4 shows analysis of MCP-1 in blood sample data according to particular exemplary aspects. Luminosity units for the leftmost two groups (the OVA challenged groups) indicate that generally values in the RDC1676-01 treated group were less than the RDC1676-00 control group as shown by the dot plot in the upper graph portion, whereas in the saline only exposed groups the cytokine level values where roughly the same, or perhaps slightly increased in the RDC1676-01 treated group.

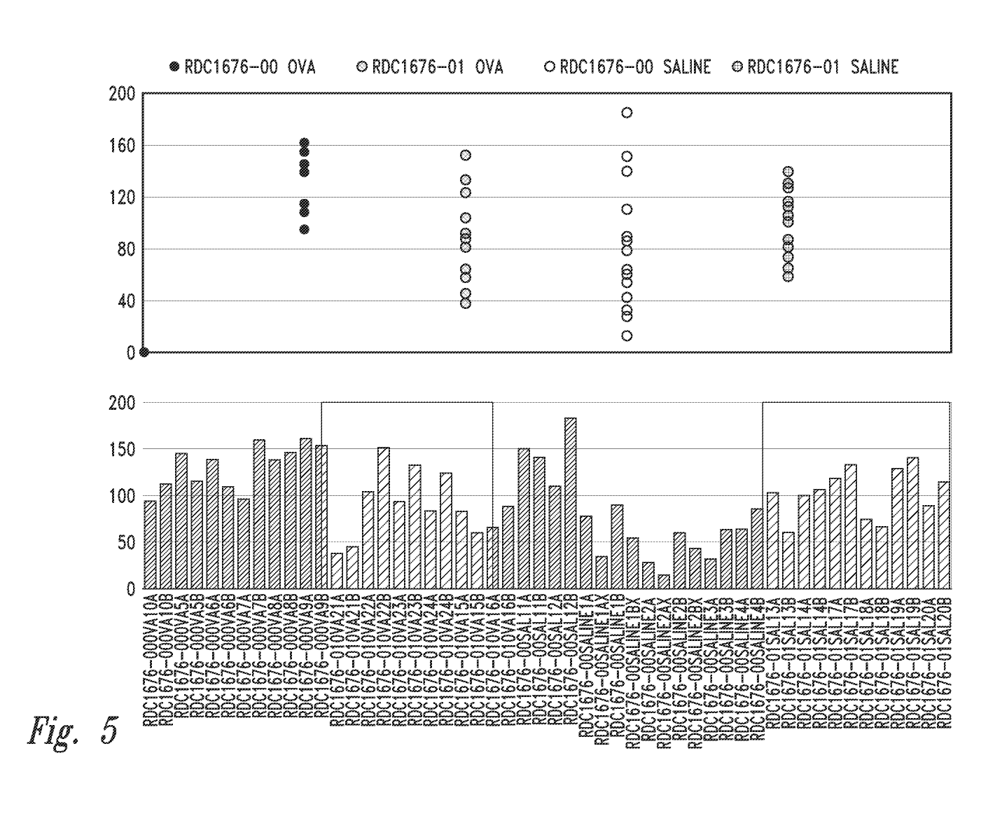

FIG. 5 shows analysis of TNF alpha in blood sample data according to particular exemplary aspects. Luminosity units for the leftmost two groups (the OVA challenged groups) indicate that generally values in the RDC1676-01 treated group were less than the RDC1676-00 control group as shown by the dot plot in the upper graph portion, whereas in the saline only exposed groups the cytokine level values where roughly the same, or perhaps slightly increased in the RDC1676-01 treated group.

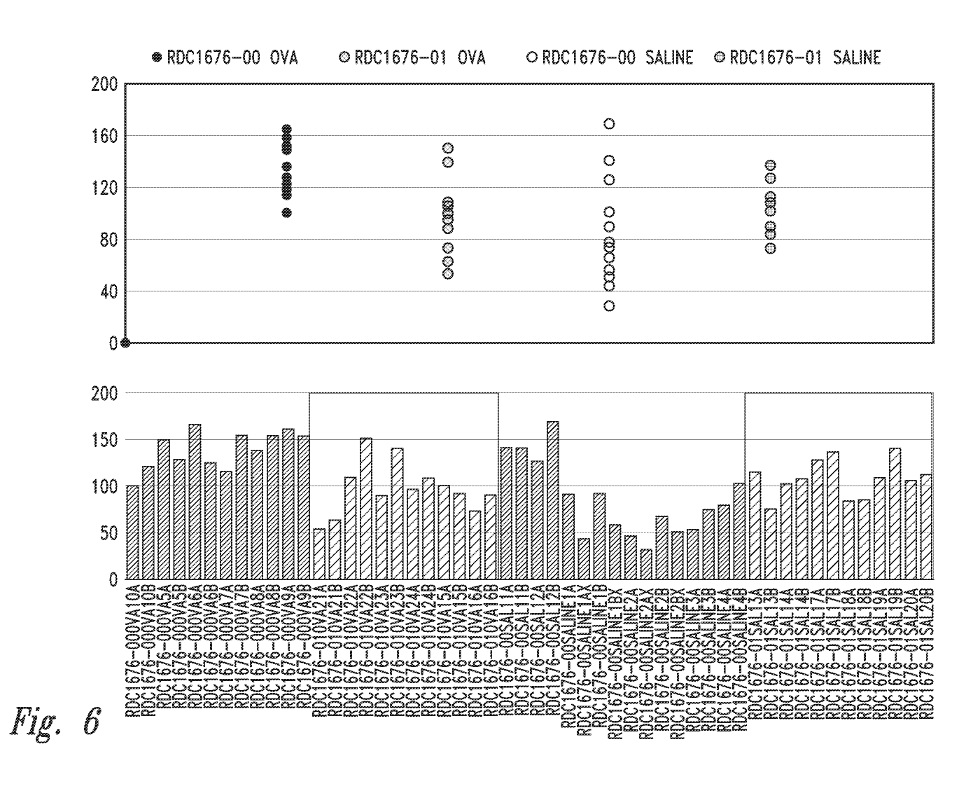

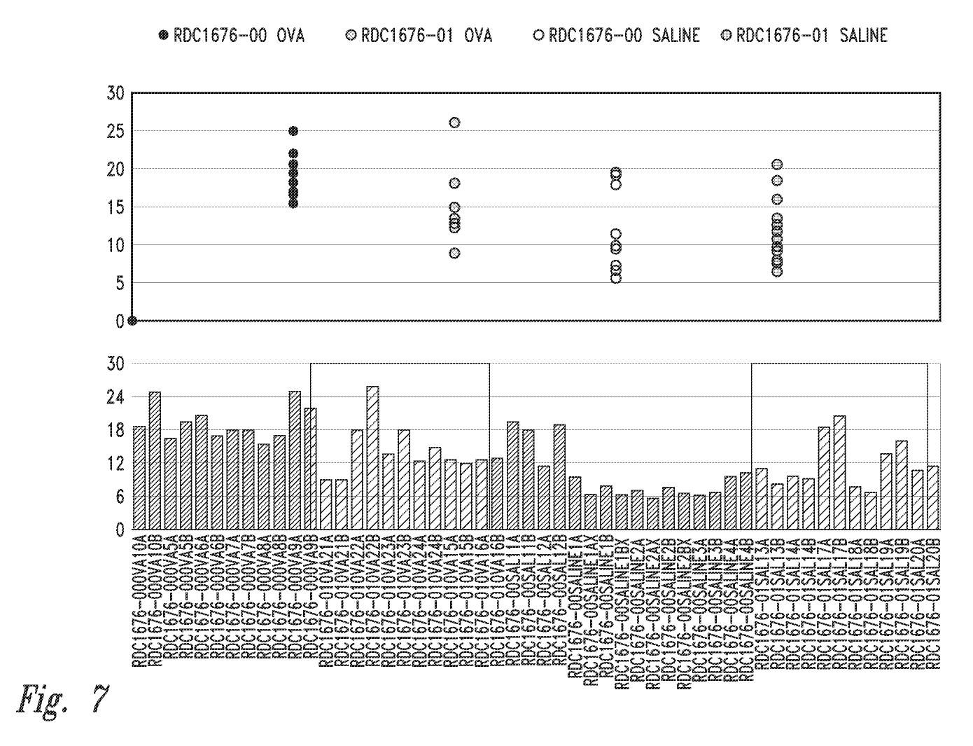

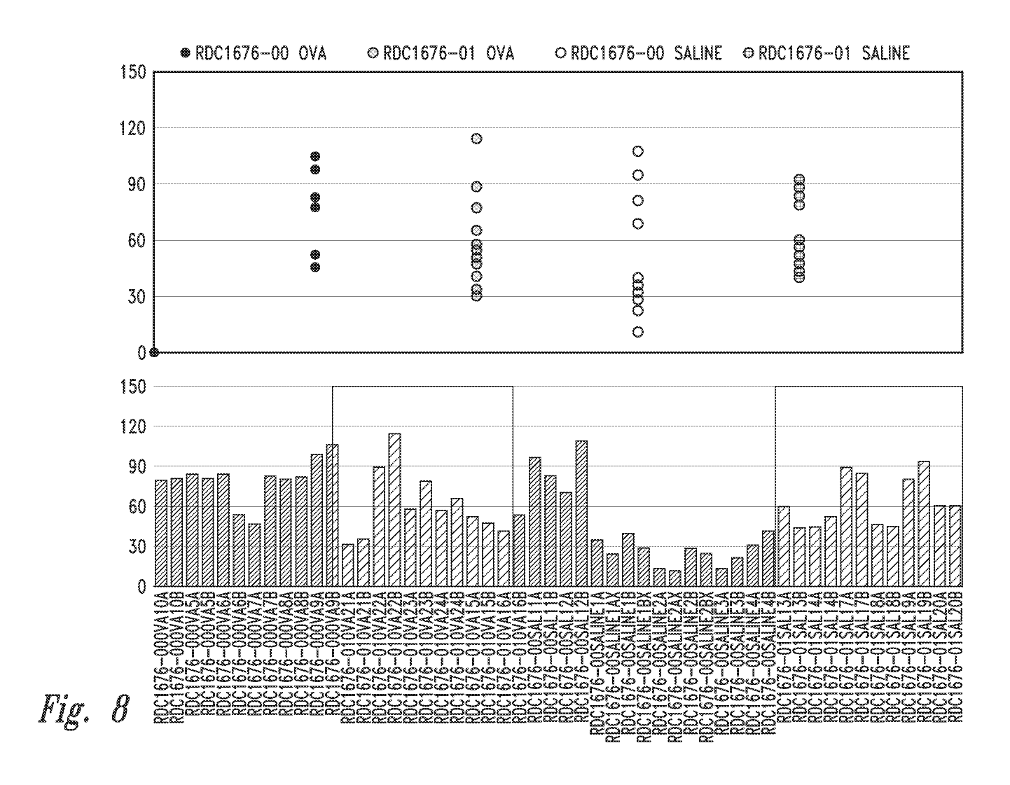

FIG. 6 shows analysis of MIP-1 alpha in blood sample data according to particular exemplary aspects. Luminosity units for the leftmost two groups (the OVA challenged groups) indicate that generally values in the RDC1676-01 treated group were less than the RDC1676-00 control group as shown by the dot plot in the upper graph portion, whereas in the saline only exposed groups the cytokine level values where roughly the same, or perhaps slightly increased in the RDC1676-01 treated group.

FIG. 7 shows analysis of IL-1 alpha in blood sample data according to particular exemplary aspects. Luminosity units for the leftmost two groups (the OVA challenged groups) indicate that generally values in the RDC1676-01 treated group were less than the RDC1676-00 control group as shown by the dot plot in the upper graph portion, whereas in the saline only exposed groups the cytokine level values where roughly the same, or perhaps slightly increased in the RDC1676-01 treated group.

FIG. 8 shows analysis of Vcam in blood sample data according to particular exemplary aspects. Luminosity units for the leftmost two groups (the OVA challenged groups) indicate that generally values in the RDC1676-01 treated group were less than the RDC1676-00 control group as shown by the dot plot in the upper graph portion, whereas in the saline only exposed groups the cytokine level values where roughly the same, or perhaps slightly increased in the RDC1676-01 treated group.

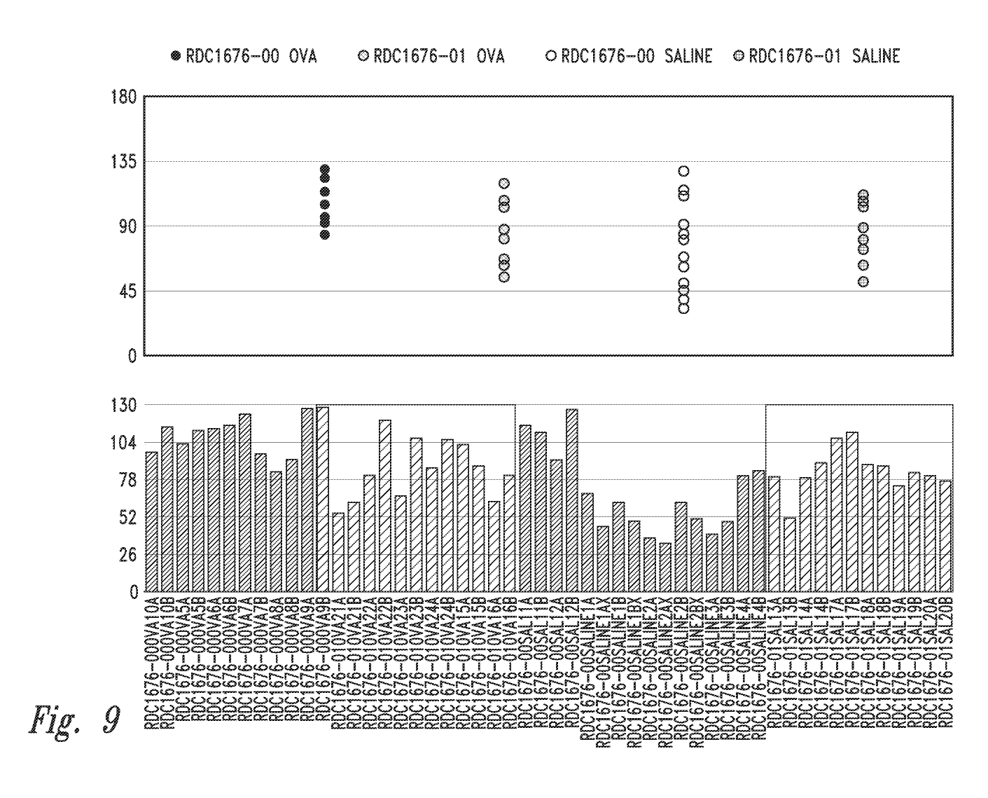

FIG. 9 shows analysis of IL-1 beta in blood sample data according to particular exemplary aspects. Luminosity units for the leftmost two groups (the OVA challenged groups) indicate that generally values in the RDC1676-01 treated group were less than the RDC1676-00 control group as shown by the dot plot in the upper graph portion, whereas in the saline only exposed groups the cytokine level values where roughly the same, or perhaps slightly increased in the RDC1676-01 treated group.

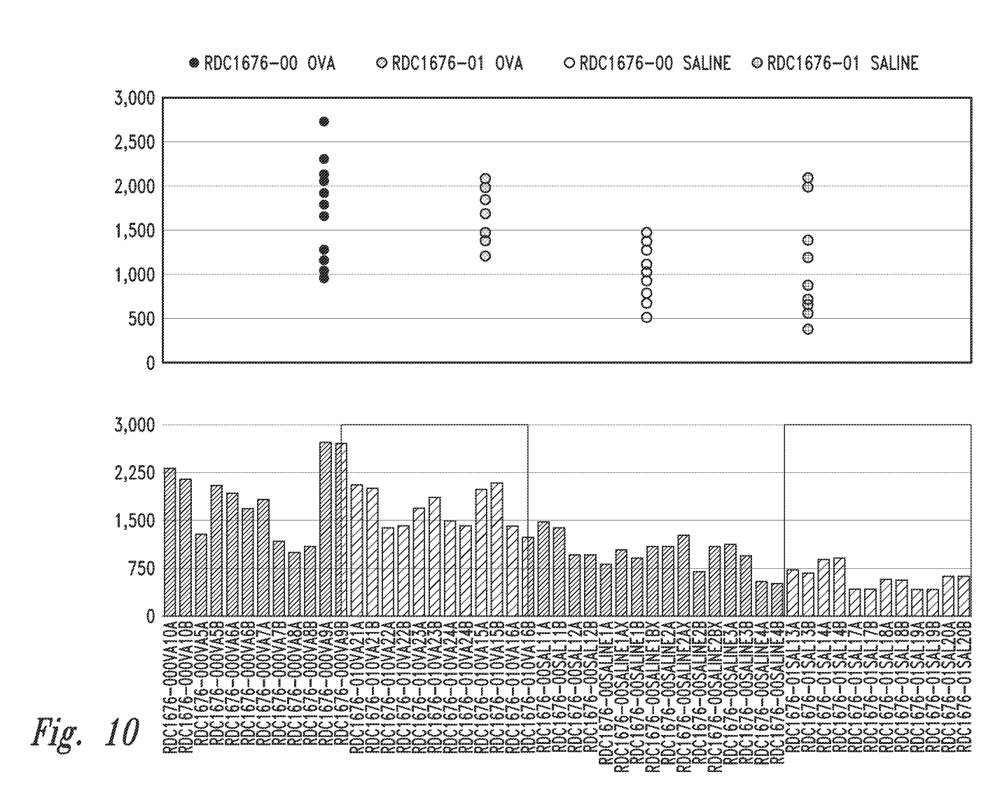

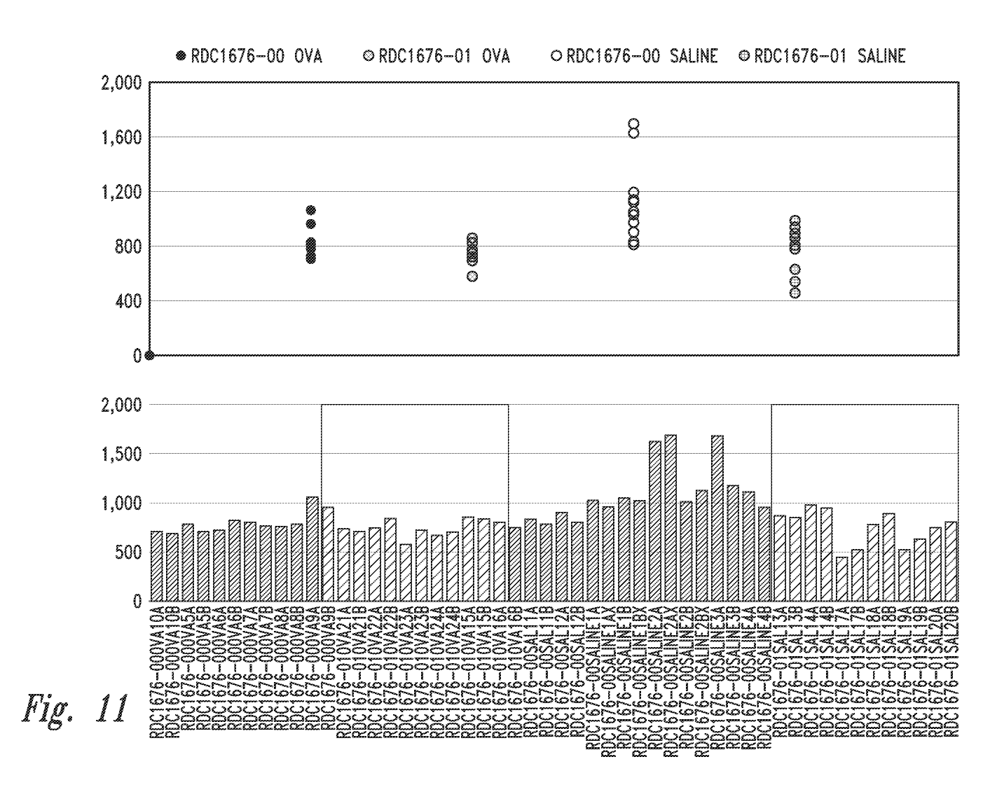

FIGS. 10 and 11 show analysis of Eotaxin and MCP-3, respectively, in blood sample data according to particular exemplary aspects. In each case, luminosity units for the leftmost two groups (the OVA challenged groups) indicate that generally values in the RDC1676-01 treated group were less than the RDC1676-00 control group as shown by the dot plot in the upper graph portion, whereas in the saline only exposed groups the cytokine level values where roughly the same, or perhaps slightly increased in the RDC1676-01 treated group.

### Bronchial Lavage Samples:

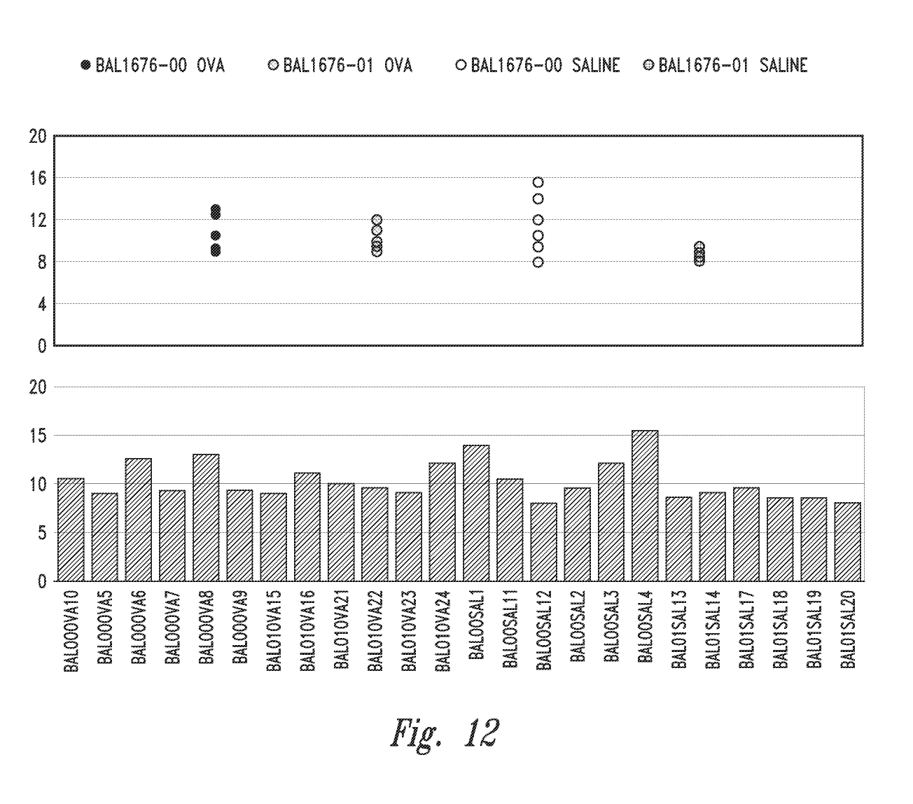

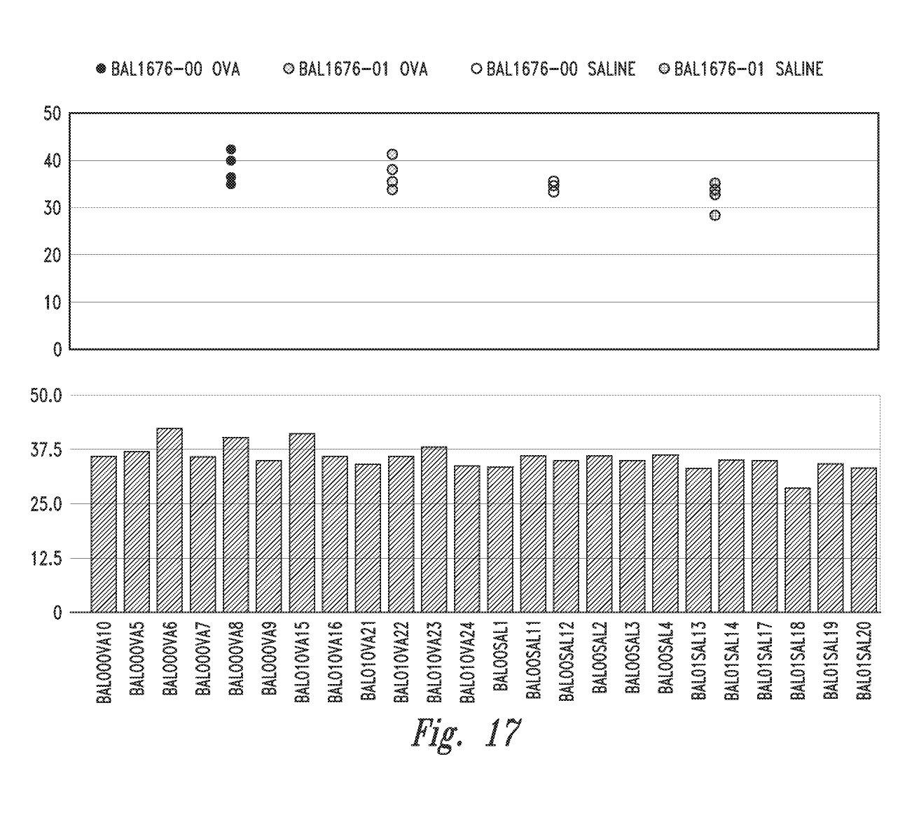

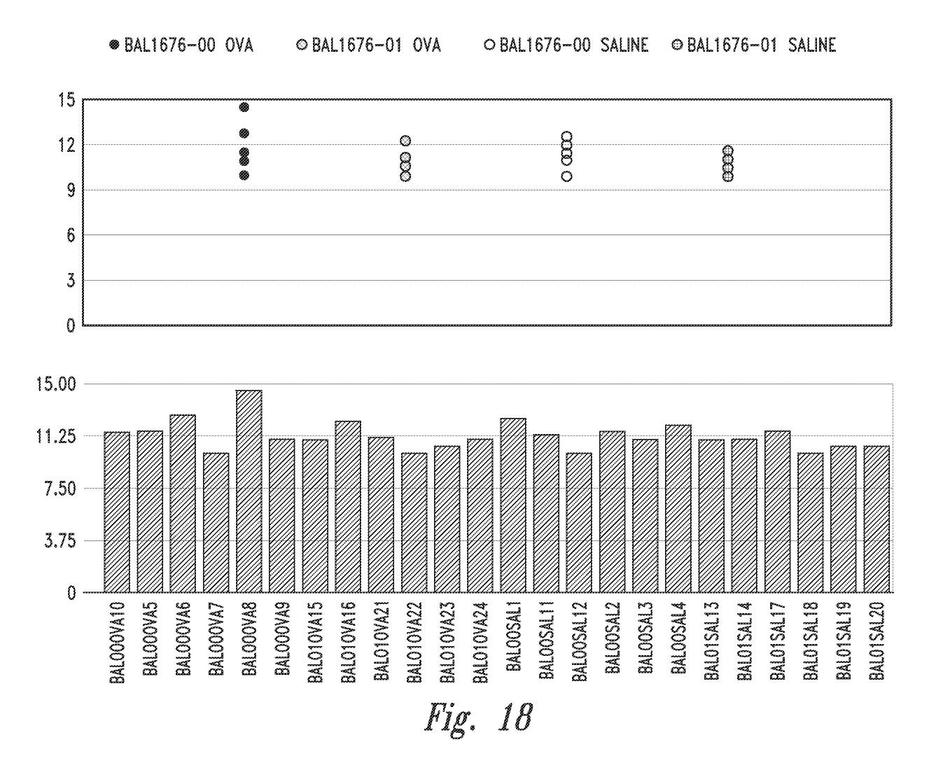

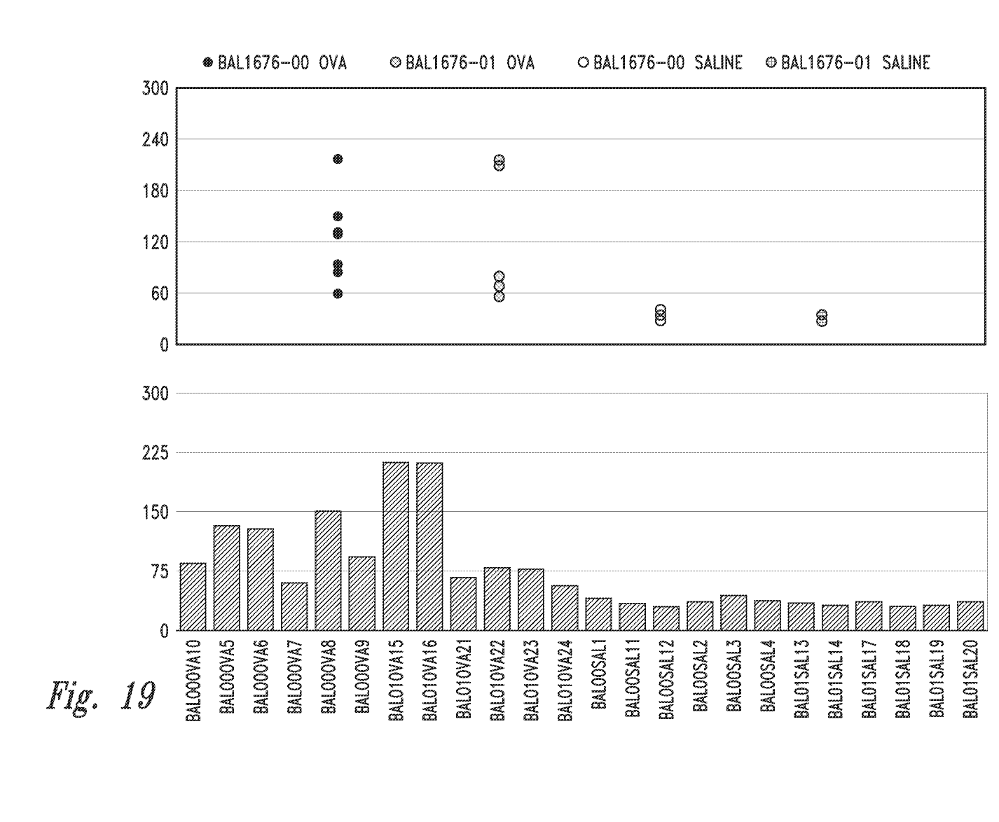

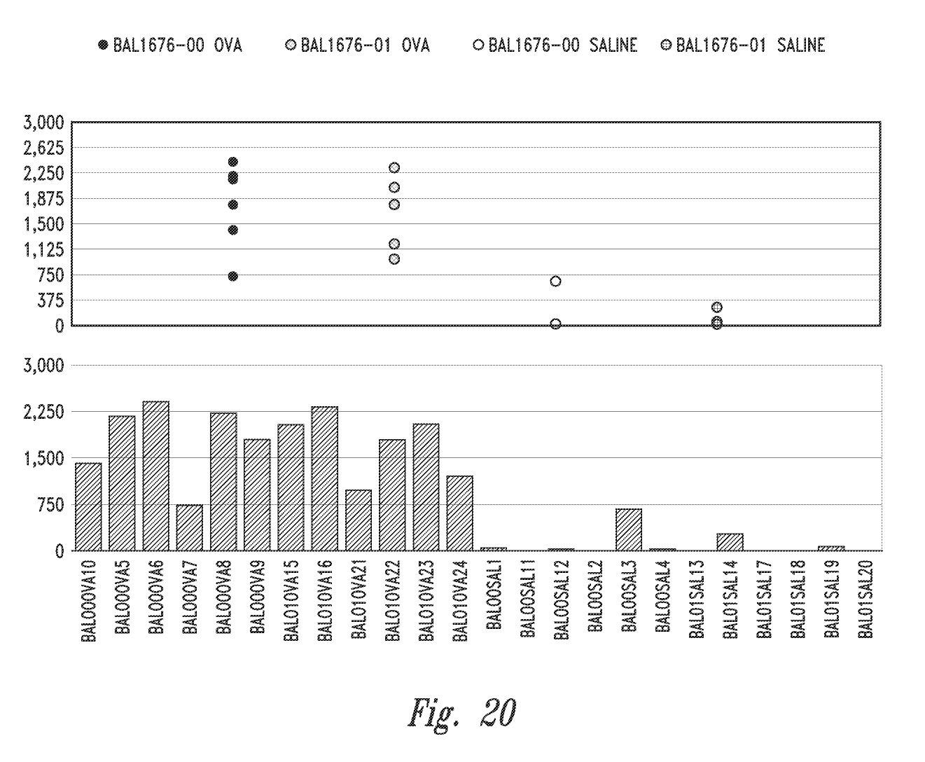

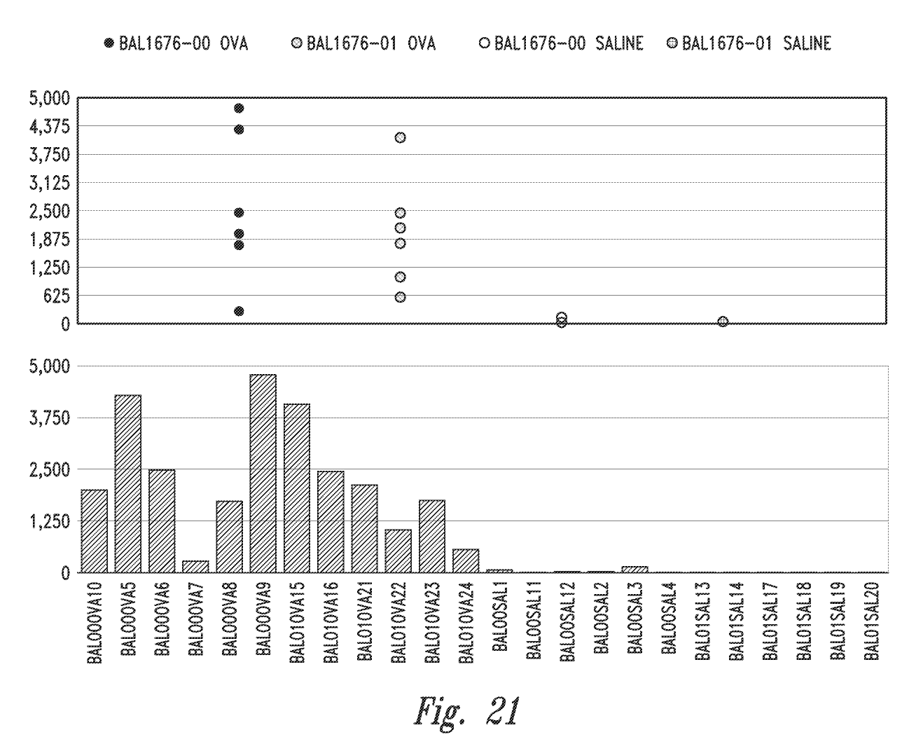

FIGS. 12-21 show the corresponding results of bronchoalveolar lavage fluid (BAL) sample evaluations.

FIG. 12 shows analysis of KC in BAL data according to particular exemplary aspects. In this instance the response level, coupled with sampling variability, was inconclusive with respect to a difference between the RDC1676-01 and RDC1676-00-treated groups; that is, KC showed relatively little difference between the 2 groups, but the units of luminosity were very small.

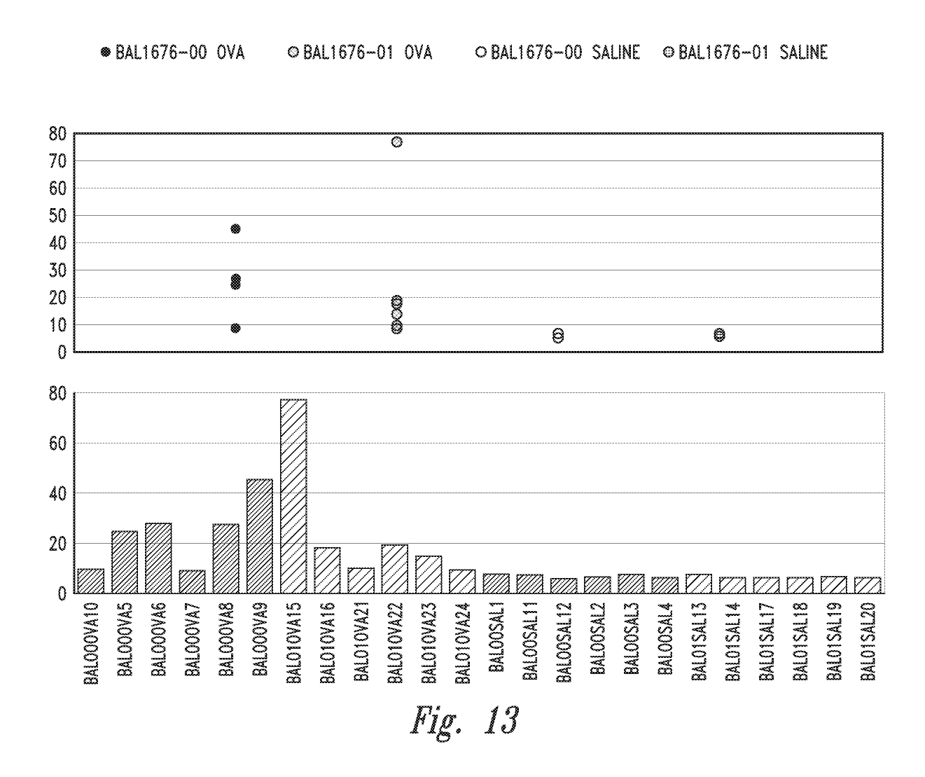

Likewise, FIG. 13 shows analysis of RANTES in BAL data according to particular exemplary aspects, and showing marked variability in the RDC1676-01 group with one reading being markedly higher than the others, skewing the results.

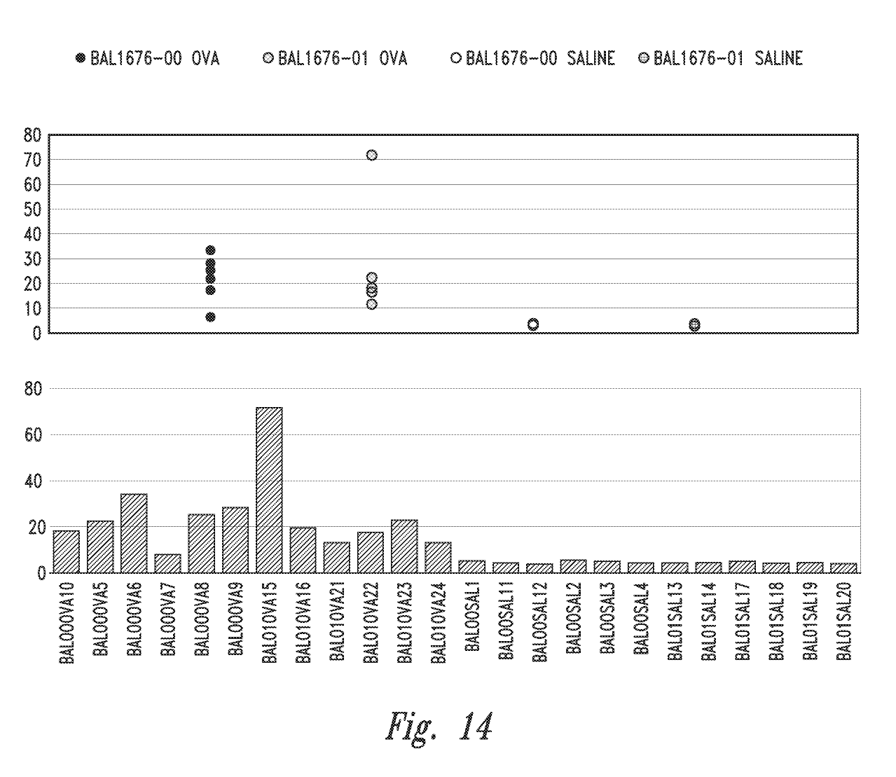

Likewise, FIG. 14 shows analysis of TNF alpha in BAL data according to particular exemplary aspects, and showing relatively little significance in the way of difference between the RDC1676-01 and RDC1676-00-treated groups.

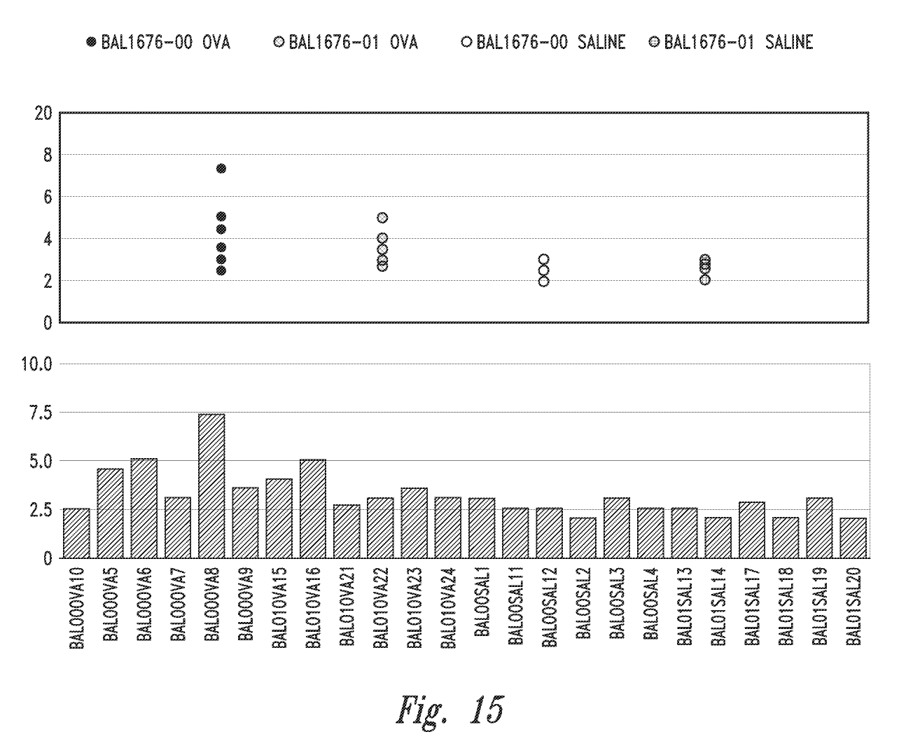

FIG. 15 shows analysis of MCP-1 in BAL data according to particular exemplary aspects, and showing relatively little significance in the way of difference between the RDC1676-01 and RDC1676-00-treated groups.

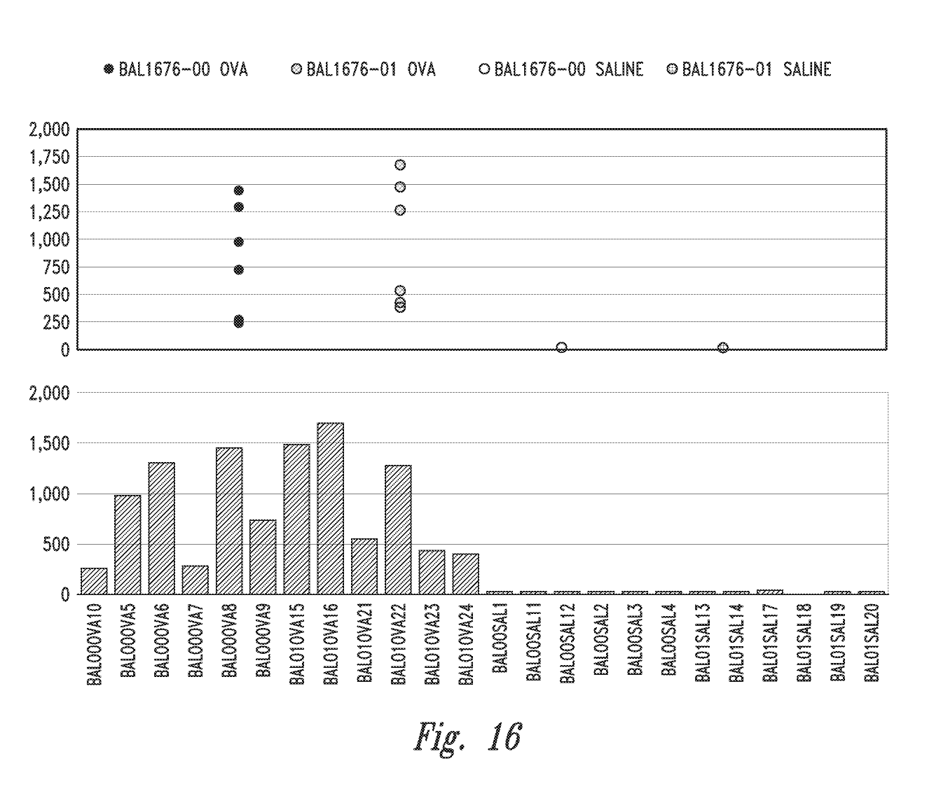

FIGS. 16 through 21 show analysis of MIP1-A, IL-1 alpha, Vcam, IL-1 beta, MCP-3, and Eotaxin, respectively, in BAL data according to particular exemplary aspects, and showing relatively little significance in the way of difference between the RDC1676-01 and RDC1676-00-treated groups.

In summary, this standard assay of inflammatory reaction to a known sensitization produced, at least in the blood samples, a marked clinical and serologic affect. Additionally, while significant numbers of control animals were physiologically stressed and nearly dying in the process, none of the RDC1676-01 treated group showed such clinical stress effects. This was reflected then in the circulating levels of cytokines, with approximately 30% differences between the RDC1676-01-treated and the RDC1676-01-treated groups in the OVA challenged groups. By contrast, there were small and fairly insignificant changes in cytokine, cellular and serologic profiles between the RDC1676-01-treated and the RDC1676-01-treated groups in the non-OVA challenged groups, which likely merely represent minimal baseline changes of the fluid itself.

### EXAMPLE 4

### Treatment of Primary Bronchial Epithelial Cells (BEG) with the Inventive Electrokinetically Generated Fluids Resulted in Reduced Expression and/or Activity of Two Key Proteins of the Airway Inflammatory Pathways, MMP9 and TSLP

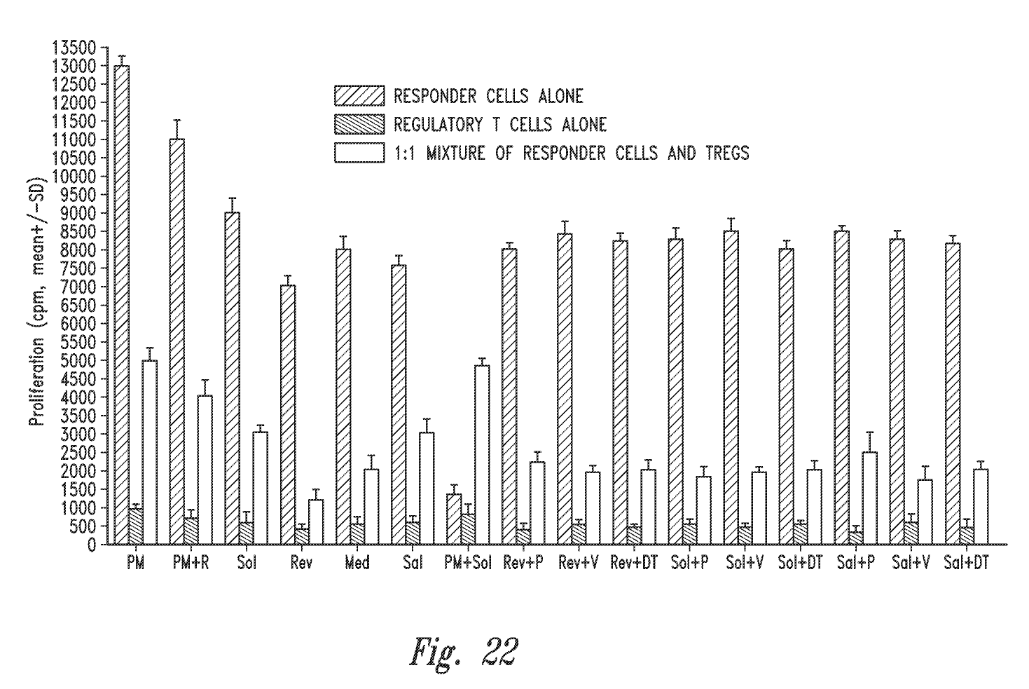

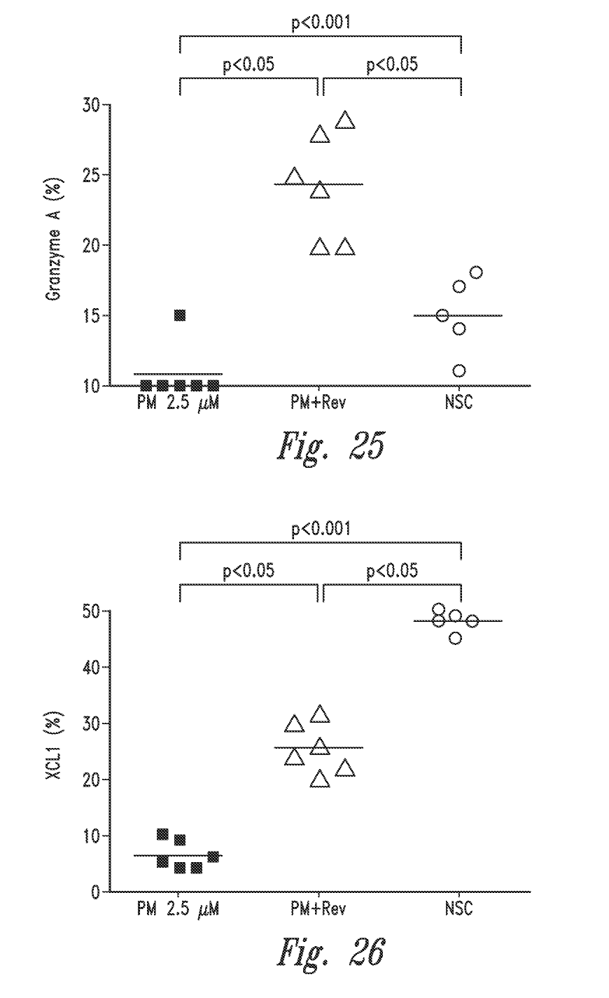

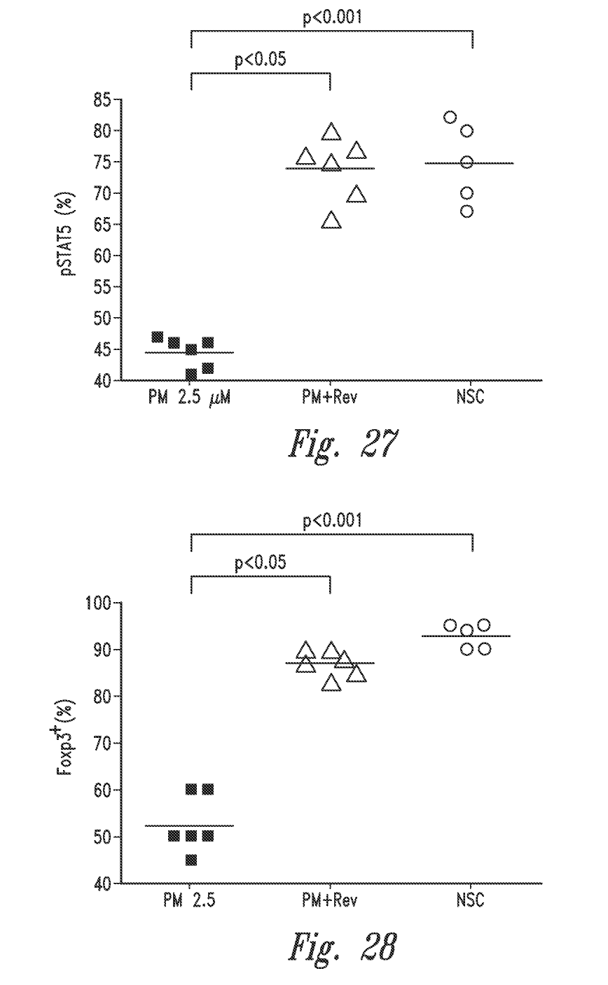

Overview. This Example is based on Example 9 of Applicants' published U.S. patent application Ser. No. 12/435,356, incorporated herein by reference for its teachings regarding modulation of inflammatory responses by electrokinetic fluids. Applicants have previously shown that Bradykinin binding to the B2 receptor was concentration dependent, and binding affinity was increased in the electrokinetically generated fluid (e.g., Rev; gas-enriched electrokinetically generated fluid) of the instant disclosure compared to normal saline. Additionally, as shown in Example 7 in the context of T-regulatory cells stimulated with diesel exhaust particulate matter (PM, standard commercial source), the data showed a decreased proliferation of T-regulatory cells in the presence of PM and Rev relative to PM in control fluid (no Rev, no Solis) (FIG. 22), indicating that the inventive electrokinetically generated fluid Rev improved regulatory T-cell function; e.g., as shown by relatively decreased proliferation in the assay. Moreover, exposure to the inventive fluids resulted in a maintained or only slightly decreased production of IL-10 relative to the Saline and Media controls (no PM). Likewise, in the context of the allergic asthma (AA) profiles of peripheral blood mononuclear cells (PBMC) stimulated with particulate matter (PM), the data showed that exposure to the fluids of the instant disclosure (“PM+Rev”) resulted in significantly lower tryptase levels similar to those of the Saline and Media controls. Additionally, the Diphtheria toxin (DT390, a truncated diphtheria toxin molecule; 1:50 dilution of std. commercial concentration) effects shown in Example 7 and FIGS. 22-29, indicate that beta blockade, GPCR blockade and Ca channel blockade affects the activity of the electrokinetically generated fluids on Treg and PBMC function. Furthermore, Applicants' previous data shows that, according to additional aspects, upon exposure to the inventive fluids, tight junction related proteins were upregulated in lung tissue. The data show upregulation of the junction adhesion molecules JAM 2 and 3, GJA1, 3, 4 and 5 (junctional adherins), OCLN (occludin), claudins (e.g., CLDN 3, 5, 7, 8, 9, 10), TJP1 (tight junction protein 1), respectively. Furthermore, the inventive electrokinetically generated fluids (e.g., RNS-60) affect modulation of whole cell conductance (e.g., under hyperpolarizing conditions) in Bronchial Epithelial Cells (BEC; e. g., Calu-3), and according to additional aspects, modulation of whole cell conductance reflects modulation of ion channels.

In this Example, Applicants have extended these discoveries by conducting additional experiments to measure the effects of production of two key proteins of the airway inflammatory pathways. Specifically, MMP9 and TSLP were assayed in primary bronchial epithelial cells (BEC).

### Materials and Methods:

Commercially available primary human bronchial epithelial cells (BEC) (HBEpC-c from Promocell, Germany) were used for these studies. Approximately 50,000 cells were plated in each well of a 12 well plate until they reached ˜80% confluence. The cells were then treated for 6 hours with normal saline, control fluid Solas or the test fluid Revera 60 at a 1:10 dilution (100 ul in 1 ml of airway epithelial growth medium) along with the diesel exhaust particulate matter (DEP or PM) before being lifted for FACS analysis. Both MMP9 and TSLP receptor antibodies were obtained from BD Biosciences and used as per manufacturer's specifications.

### Results:

Applicants show that the test material Revera 60 reduces DEP induced TSLP receptor expression in bronchial epithelial cells (BEC) by approximately 90%. Solas resulted in a 55% reduction in TSLP receptor expression, while Normal saline failed to produce similar level of reduction in TSLP receptor expression (approximately 20% reduction). The effect of the inventive solution in reducing TSLP receptor expression is a significant discovery in view of recent findings showing that TSLP plays a pivotal role in the pathobiology of allergic asthma and local antibody mediated blockade of TSLP receptor function alleviated allergic disease (Liu, Y J, Thymic stromal lymphopoietin: Master switch for allergic inflammation, J Exp Med 203:269-273, 2006; Al-Shami et al., A role for TSLP in the development of inflammation in an asthma model, J Exp Med 202:829-839, 2005; and Shi et al., Local blockade of TSLP receptor alleviated allergic disease by regulating airway dendritic cells, Clin Immunol. 2008, Aug. 29. (Epub ahead of print)).

Likewise, FIG. 39 shows the effect of Revera 60, Solas and normal saline on the DEP-mediated increase in MMP 9. Specifically, Revera 60 inhibited the DEP-induced cell surface bound MMP9 levels in bronchial epithelial cells by approximately 80%, and Solas had an inhibitory effect of approximately 70%, whereas normal saline (NS) had a marginal effect of about 20% reduction. MMP-9 is one of the major proteinases involved in airway inflammation and bronchial remodeling in asthma. Recently, it has been demonstrated that the levels of MMP-9 are significantly increased in patients with stable asthma and even higher in acute asthmatic patients compared with healthy control subjects. MMP-9 plays a crucial role in the infiltration of airway inflammatory cells and the induction of airway hyperresponsiveness indicating that MMP-9 may have an important role in inducing and maintaining asthma (Vignola et al., Sputum metalloproteinase-9/tissue inhibitor of metalloproteinase-1 ratio correlates with airflow obstruction in asthma and chronic bronchitis, Am J Respir Crit Care Med 158:1945-1950, 1998; Hoshino et al., Inhaled corticosteroids decrease subepithelial collagen deposition by modulation of the balance between matrix metalloproteinase-9 and tissue inhibitor of metalloproteinase-1 expression in asthma, J Allergy Clin Immunol 104:356-363, 1999; Simpson et al., Differential proteolytic enzyme activity in eosinophilic and neutrophilic asthma, Am J Respir Crit Care Med 172:559-565,2005; Lee et al., A murine model of toluene diisocyanate-induced asthma can be treated with matrix metalloproteinase inhibitor, J Allergy Clin Immunol 108:1021-1026, 2001; and Lee et al., Matrix metalloproteinase inhibitor regulates inflammatory cell migration by reducing ICAM-1 and VCAM-1 expression in a murine model of toluene diisocyanate-induced asthma, J Allergy Clin Immunol 2003;111:1278-1284).

According to additional aspects, therefore, the inventive electrokinetically generated fluids have substantial therapeutic utility for modulating (e.g., reducing) TSLP receptor expression and/or for inhibiting expression and/or activity of MMP-9, including, for example, for treatment of inflammation and asthma.

### EXAMPLE 5

### The Inventive Electrokinetically Generated Fluids were Shown to have a Synergistic Anti-Inflammatory Effect with Budesonide in an Art-Recognized Animal Model for Allergic Asthma

This Example is based on Example 10 of Applicants' published U.S. patent application Ser. No. 12/435,356, incorporated herein by reference for its teachings regarding modulation of inflammatory responses by electrokinetic fluids in the context of an asthma model. This working Example describes experiments performed to assess the airway anti-inflammatory properties of the inventive electrokinetically generated fluids (e.g., RDC-1676-03) in a Brown Norway rat ovalbumin sensitization model. The Brown Norway rat is an art-recognized model for determining the effects of a test material on airway function and this strain has been widely used, for example, as a model of allergic asthma. Airway pathology and biochemical changes induced by ovalbumin sensitization in this model resemble those observed in man (Elwood et al., J Allergy Clin Immuno 88:951-60, 1991; Sirois & Bissonnette, Clin Exp Immunol 126:9-15, 2001). The inhaled route was selected to maximize lung exposure to the test material or the control solution. The ovalbumin-sensitized animals were treated with budesonide alone or in combination with the test material RDC 1676-03 for 7 days prior to ovalbumin challenge. 6 and 24 hours following the challenge, total blood count and levels of several pro and anti-inflammatory cytokines as well as various respiratory parameters were measured to estimate any beneficial effect of administering the test material on various inflammatory parameters.

### Materials and Methods:

Brown Norway rats of strain Bn/Crl were obtained from Charles River Kingston, weighing approximately 275±50 g at the onset of the experiment. All animal studies were conducted with the approval by PCS-MTL Institutional Animal Care and Use Committee. During the study, the use and care of animals were conducted according to guidelines of the USA National Research Council as well as Canadian Council of Animal Care.

Sensitization. On day 1 of the experiment, animals (14 animals in each treatment group) were sensitized by administration of a 1 ml intraperitoneal injection of a freshly prepared solution of 2 mg ovalbumin/100 mg Aluminum Hydroxide per 1 ml of 0.9% Sodium Chloride, followed by repeat injection on day 3.

Treatment. Fifteen days following the initial sensitization, animals were subjected to nebulized exposure to control (Normal saline) or test solutions (electrokinetically generated fluids RDC1676-00, RDC1676-02 and RDC-1676-03), either administered alone or in combination with Budesonide, once daily for 15 minutes for 7 consecutive days. Animals were dosed in a whole body chamber of approximately 20 L, and test atmosphere was generated into the chamber air inlet using aeroneb ultrasonic nebulizers supplied with air from a Buxco bias flow pump. The airflow rate was set at 10 liters/min.

Ovalbumin challenge. On day 21, 2 hours following treatment with the test solutions, all animals were challenged with 1% ovalbumin nebulized solution for 15 minutes (in a whole body chamber at airflow 2 L/min).

Sample collection. At time points of 6 and 24 hours after the ovalbumin challenge, blood samples were collected for total and differential blood cell counts as well as for measuring levels of various pro and anti-inflammatory cytokines. In addition, immediately after and at 6 and 24 hours following ovalbumin challenge the enhanced pause Penh and tidal volume were measured for a period of 10 minutes using the Buxco Electronics BioSystem XA system.

### Results:

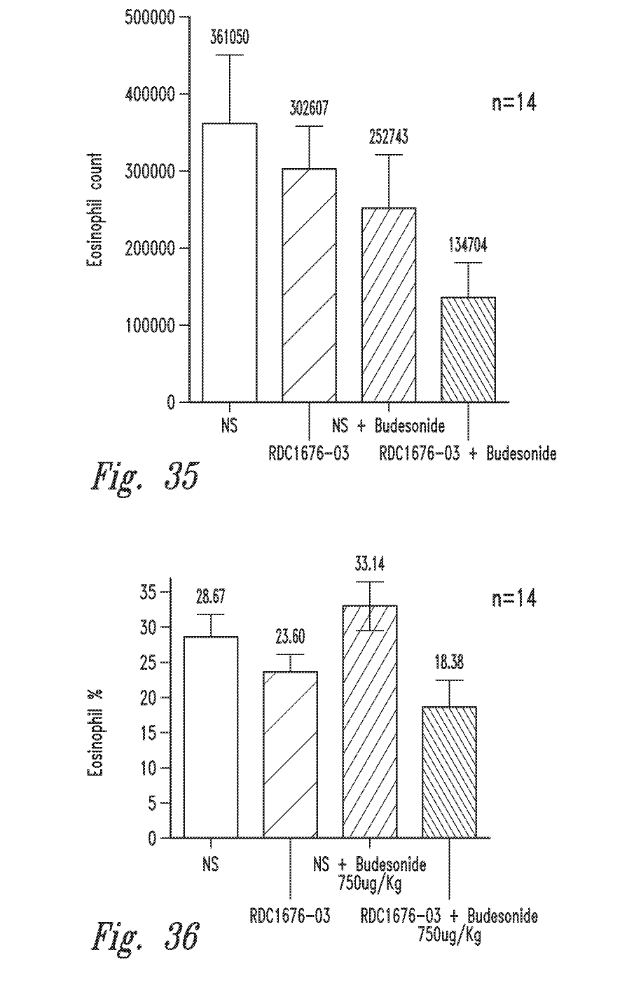

Eosinophil Count: As expected, and shown in FIG. 35, treatment with Budesonide (“NS+Budesonide 750 μg/Kg”; densely crosshatched bar graph) reduced the total eosinophil count in the challenged animals relative to treatment with the normal saline “NS” alone control (open bar graph). Additionally, while treatment with the inventive fluid “RDC1676-03” alone (lightly crosshatched bar graph) did not significantly reduce the eosinophil count, it nonetheless displayed a substantial synergy with Budesonide in reducing the eosinophil count (“RDC1676-03+Budesonide 750 μg/Kg”, solid dark bar graph). Similarly, in FIG. 36, the Eosinophil % also reflected a similar trend. While RDC1676-03 (lightly crosshatched graph bar) or Budesonide 750 ug/kg (densely crosshatched bar graph) alone did not have a significant effect on Eosinophil % count in the challenged animals, the two in combination reduced the Eosinophil % significantly (solid dark bar graph).

Therefore, FIGS. 35 and 36 show, according to particular aspects of the present invention that the inventive electrokinetically generated fluids (e.g., RDC1676-03) were demonstrated to have a substantial synergistic utility in combination with Budesonide to significantly reduce eosinophil count (“Eosinophil %” and total count) in an art-recognized rat model for human allergic asthma.

### Respiratory Parameters:

Applicants demonstrate the observed effect of the test fluids on Penh and tidal volume as measured immediately, 6 and 24 hours after the ovalbumin challenge. Penh is a derived value obtained from peak inspiratory flow, peak expiratory flow and time of expiration and lowering of penh value reflects a favorable outcome for lung function.

Penh=(Peak expiratory flow/Peak inspiratory flow)*(Expiratory time/time to expire 65% of expiratory volume−1).

Treatment with Budesonide (at both 500 and 750 ug/kg) alone or in combination with any of the test fluids failed to significantly affect the Penh values immediately after the challenge. However, 6 hours after the challenge, animals treated with RDC1676-03 alone or in combination with Budesonide 500 or 750 ug/kg demonstrated a significant drop in Penh values. Although the extent of this drop was diminished by 24 hours post challenge, the trend of a synergistic effect of Budesonide and RDC fluid was still observed at this time point.

Tidal volume is the volume of air drawn into the lungs during inspiration from the end-expiratory position, which leaves the lungs passively during expiration in the course of quiet breathing. Animals treated with Budesonide alone showed no change in tidal volumes immediately after the challenge. However, RDC 1676-03 alone had a significant stimulatory effect on tidal volume even at this early time point. And again, RDC1676-03 in combination with Budesonide (both 500 and 750 ug/kg) had an even more pronounced effect on Tidal volume measurements at this time point. Six hours after the challenge, RDC1676-03 alone was sufficient to cause a significant increase in tidal volume and addition of Budesonide to the treatment regimen either alone or in combination had no added effect on tidal volume. Any effect observed at these earlier time points were, however, lost by the 24 hours time point.

Taken together, these data demonstrate that RDC1676-03 alone or in combination with Budesonide provided significant relief to airway inflammation as evidenced by increase in tidal volume and decrease in Penh values at 6 hours post challenge.

### Cytokine Analysis:

To analyze the mechanism of the effects seen on the above discussed physiological parameters, a number of pro as well as anti-inflammatory cytokines were measured in blood samples collected at 6 and 24 hours after the challenge, immediately following the physiological measurements.

FIGS. 37A and 37B clearly demonstrate that Rev 60 (or RDC1676-03) alone lowered the blood level of eotaxin significantly at both 6 and 24 hours post challenge. Budesonide 750 ug/kg also reduced the blood eotaxin levels at both of these time points, while Budesonide 250 ug/kg only had a notable effect at the later time point. However, the test solution Rev 60 alone showed effects that are significantly more potent (in reducing blood eotaxin levels) than both concentrations of Budesonide, at both time points. Eotaxin is a small C—C chemokine known to accumulate in and attract eosinophils to asthmatic lungs and other tissues in allergic reactions (e.g., gut in Crohn's disease). Eotaxin binds to a G protein coupled receptor CCR3. CCR3 is expressed by a number of cell types such as Th2 lymphocytes, basophils and mast cells but expression of this receptor by Th2 lymphocyte is of particular interest as these cells regulate eosinophil recruitment. Several studies have demonstrated increased production of eotaxin and CCR3 in asthmatic lung as well as establishing a link between these molecules and airway hyperresponsiveness (reviewed in “Eotaxin and the attraction of eosinophils to the asthmatic lung,” Dolores M Conroy and Timothy J Williams Respiratory Research 2001, 2:150-156). It is of particular interest to note that these studies completely agree with the results in FIGS. 35 and 36 on eosinophil counts.

Taken together these results strongly indicate that treatment with RDC1676-03 alone or in combination with Budesonide can significantly reduce eosinophil total count and % in blood 24 hours after the ovalbumin challenge. This correlates with a significant drop in eotaxin levels in blood observed as early as 6 hours post challenge.

Blood levels of two major key anti-inflammatory cytokines, IL10 and Interferon gamma are also significantly enhanced at 6 hours after challenge as a result of treatment with Rev 60 alone or in combination with Budesonide. FIGS. 37C and 37D show such effects on Interferon gamma and IL10, respectively. It is evident from these figures that Rev 60 alone or Rev 60 in combination with Budesonide 250 ug/kg significantly increased the blood level of IL10 in the challenged animals up to 6 hrs post challenge. Similarly, Rev 60 alone or in combination with Budesonide 250 or 750 ug/kg significantly increased the blood level of IFN gamma at 6 hours post challenge. Increase in these anti-inflammatory cytokines may well explain, at least in part, the beneficial effects seen on physiological respiratory parameters seen 6 hours post challenge. The effect on these cytokines was no longer observed at 24 hour post challenge (data not shown).

Rantes or CCL5 is a cytokine expressed by circulating T cells and is chemotactic for T cells, eosinophils and basophils and has an active role in recruiting leukocytes into inflammatory sites. Rantes also activates eosinophils to release, for example, eosinophilic cationic protein. It changes the density of eosinophils and makes them hypodense, which is thought to represent a state of generalized cell activation. It also is a potent activator of oxidative metabolism specific for eosinophils.

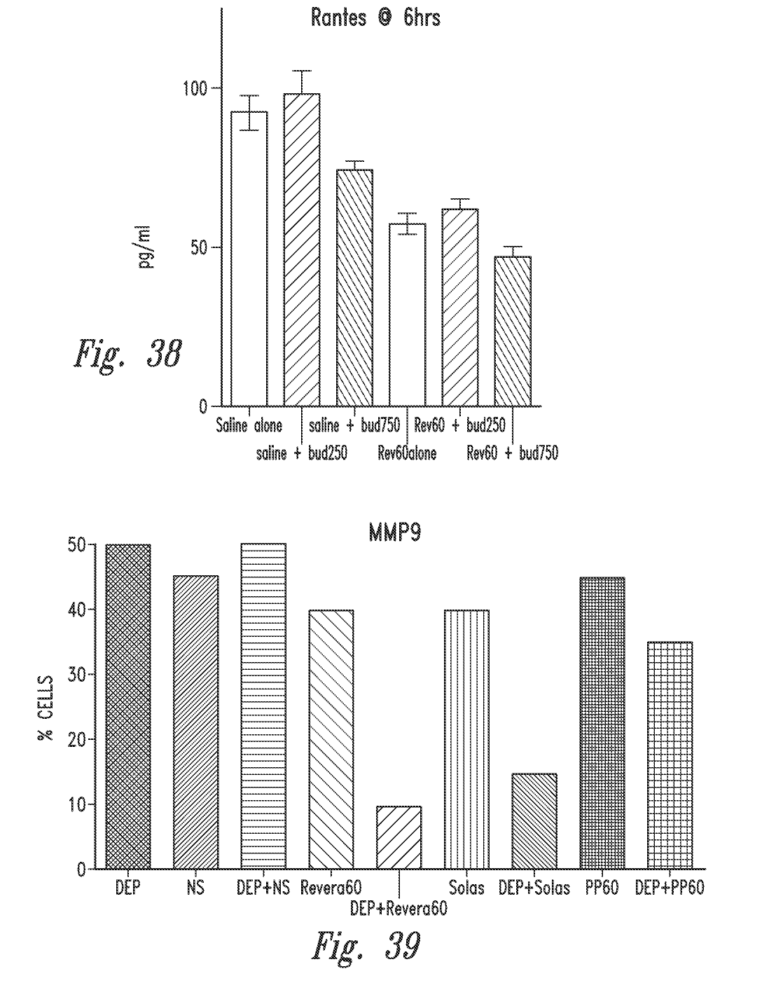

As shown in FIG. 38, systemic levels of Rantes was reduced significantly at 6 hours, but not at 24 hours post challenge in animals treated with Rev 60 alone or in combination of Budesonide 250 or 750 ug/kg. Once again, there is a clear synergistic effect of Budesonide 750 ug/kg and Rev 60 that is noted in this set of data. A similar downward trend was observed for a number of other pro-inflammatory cytokines, such as KC or IL8, MCP3, IL1b, GCSF, TGFb as well as NGF, observed either at 6 or at 24 hours post challenge, in animals treated with Rev60 alone or in combination with Budesonide.

### EXAMPLE 6

### The Inventive Therapeutic Fluids have Substantial Utility for Modulating Nitric Oxide Levels

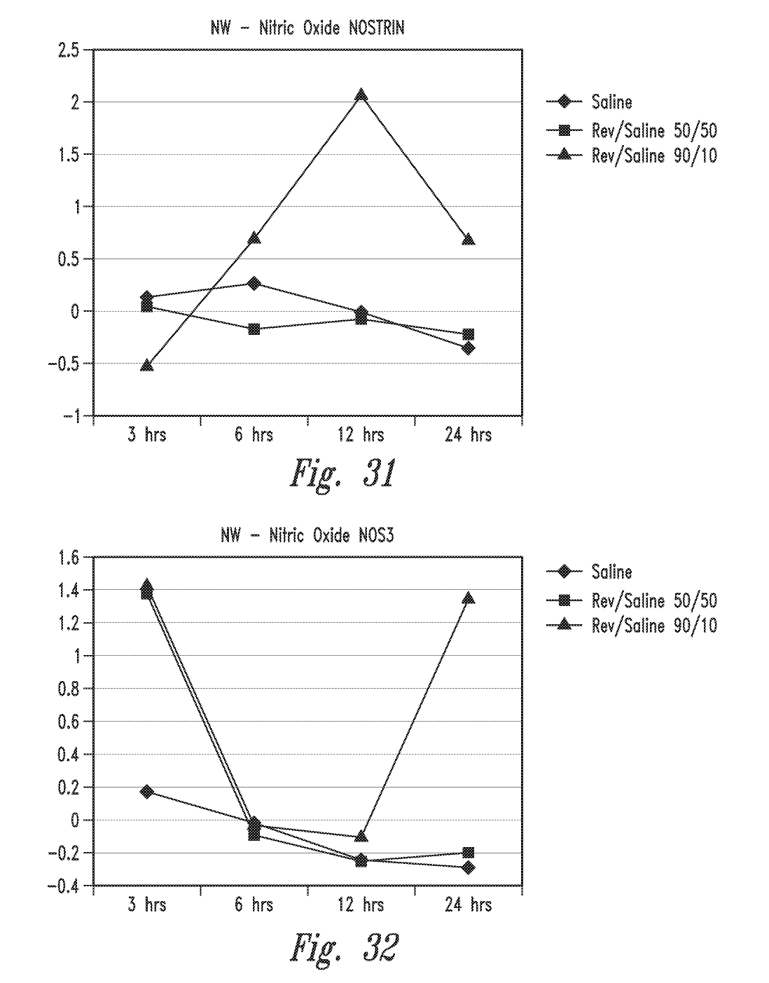

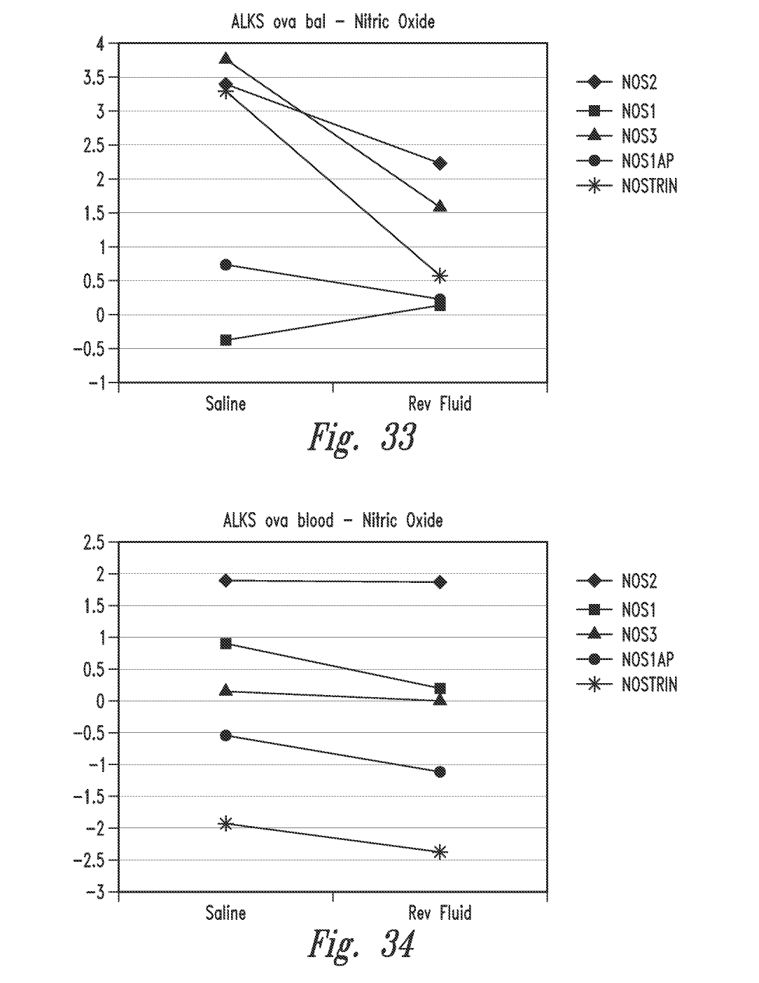

This Example is based on Example 12 of Applicants' published U.S. patent application Ser. No. 12/435,356, incorporated herein by reference for its teachings regarding modulation of nitric oxide levels by electrokinetic fluids. According to particular aspects, the inventive diffuser processed therapeutic fluids have substantial utility for modulating nitric oxide levels, and/or related enzymes. FIGS. 30-34 show data obtained from human foreskin keratinocytes exposed to RDC1676-01 (sterile saline processed through the instant proprietary device with additional oxygen added; gas-enriched electrokinetically generated fluid (Rev) of the instant disclosure) showing up-regulation of NOS1 and 3, and Nostrin, NOS3. By contrast, data obtained from rat lung tissue (tissue of above Example entitled “Cytokine Expression”) shows down regulation of NOS2 and 3, Nostrin and NOS1AP with Rev (FIGS. 33 and 34).

### EXAMPLE 7

### A Regulatory T-Cell Assay was Used to Show Effects of the Inventive Electrokinetically Generated Fluids in Modulation of T-Cell Proliferation and Elaboration of Cytokines (Il-10) and other Proteins (e.g., GITR, Granzyme A, XCL1, pStat5, and Foxp3)) in Regulatory T-Cell Assays, and of for Example, Tryptase in PBMC

This Example is based on Example 8 of Applicants' published U.S. patent application Ser. No. 12/435,356, incorporated herein by reference for its teachings regarding modulation of inflammation by electrokinetic fluids. The ability of particular embodiments disclosed herein to regulate T cells was studied by irradiating antigen presenting cells, and introducing antigen and T cells. Typically, these stimulated T cells proliferate. However, upon the introduction of regulatory T cells, the usual T cell proliferation is suppressed.

### Methods:

Briefly, FITC-conjugated anti-CD25 (ACT-1) antibody used in sorting was purchased from DakoCytomation (Chicago, Ill.). The other antibodies used were as follows: CD3 (HIT3a for soluble conditions), GITR (PE conjugated), CD4 (Cy-5 and FITC-conjugated), CD25 (APC-conjugated), CD28 (CD28.2 clone), CD127-APC, Granzyme A (PE-conjugated), FoxP3 (BioLegend), Mouse IgG1 (isotype control), and XCL1 antibodies. All antibodies were used according to manufacturer's instructions. CD4+ T cells were isolated from peripheral whole blood with CD4+ Rosette Kit (Stemcell Technologies). CD4+ T cells were incubated with anti-CD127-APC, anti-CD25-PE and anti-CD4-FITC antibodies. Cells were sorted by flow cytometry using a FACS Aria into CD4+CD25hiCD1271o/nTreg and CD4+CD25-responder T cells.

Suppression assays were performed in round-bottom 96 well microtiter plates. 3.75×103 CD4+CD25neg responder T cells, 3.75×103 autologous T reg, 3.75×104 allogeneic irradiated CD3-depleted PBMC were added as indicated. All wells were supplemented with anti-CD3 (clone HIT3a at 5.0 μg/ml). T cells were cultured for 7 days at 37° C. in RPMI 1640 medium supplemented with 10% fetal bovine serum. Sixteen hours before the end of the incubation, 1.0 mCi of ³H-thymidine was added to each well. Plates were harvested using a Tomtec cell harvester and ³H-thymidine incorporation determined using a Perkin Elmer scintillation counter. Antigen-presenting cells (APC) consisted of peripheral blood mononuclear cells (PBMC) depleted of T cells using StemSep human CD3+ T cell depletion (StemCell Technologies) followed by 40 Gy of irradiation.

Regulatory T cells were stimulated with anti-CD3 and anti-CD28 conditions and then stained with Live/Dead Red viability dye (Invitrogen), and surface markers CD4, CD25, and CD127. Cells were fixed in the Lyze/Fix PhosFlow™ buffer and permeabilized in denaturing Permbuffer III®. Cells were then stained with antibodies against each particular selected molecule.

Statistical analysis was performed using the GraphPad Prism software. Comparisons between two groups were made by using the two-tailed, unpaired Student's t-test. Comparisons between three groups were made by using 1-way ANOVA. P values less than 0.05 were considered significant (two-tailed). Correlation between two groups were determined to be statistically significant via the Spearman coefficient if the r value was greater than 0.7 or less than −0.7 (two-tailed).

### Results:

As indicated in FIG. 22, regulatory T cell proliferation was studied by stimulating cells with diesel exhaust particulate matter (PM, from EPA). The x-axis of FIG. 22 shows activated autologous CD4+ effector T cells (responder cells) as a solid black bar, and regulatory T cells alone in the gray bar (shown for confirmation of anergy) which were mixed at a 1:1 ratio as shown in the white bar. The y axis shows proliferation as measured by uptake of ³H-thymidine. As shown from left to right along the x-axis, “PM” indicates diesel exhaust derived Particulate Matter, “PM+Rev” indicates PM plus a gas-enriched electrokinetically generated fluid (Rev) of the instant disclosure, “Solis” indicates an electrokinetically generated fluid of the instant disclosure and device that is not gas-enriched beyond ambient atmosphere, only (no PM added), “Rev” indicates Rev alone (no PM added) as defined above, “Media” indicates the cell growth media alone control (minus PM; no Rev, no Solis), and “Saline Con” indicates the saline control (minus PM; no Rev, no Solis), “V” indicates verapamil, and “P” indicates propanolol, and “DT” is DT390 at 1:50.

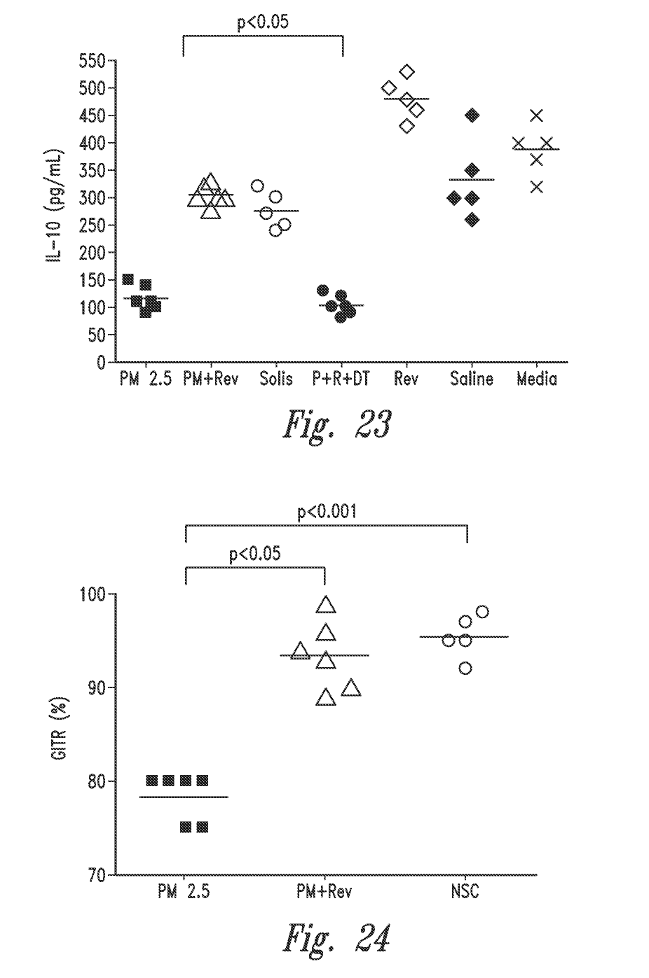

As shown in FIG. 23, cells stimulated with PM (no Rev, no Solis) resulted in a decrease in secreted IL-10, while cells exposed to PM in the presence of the fluids of the instant disclosure (“PM+Rev”) resulted in a maintained or only slightly decreased production of IL-10 relative to the Saline and Media controls (no PM). Furthermore, Diphtheria toxin (DT390, a truncated diphtheria toxin molecule; 1:50 dilution of std. commercial concentration) was titrated into inventive fluid samples, and blocked the Rev-mediated effect of increase in IL-10 in FIG. 23. Note that treatment with Rev alone resulted in higher IL-10 levels relative to Saline and Media controls.

Likewise, similar results, shown in FIGS. 24-28, were obtained with GITR, Granzyme A, XCL1, pStat5, and Foxp3, respectively. In Figures, “NSC” is the same as “Solis” (no PM).

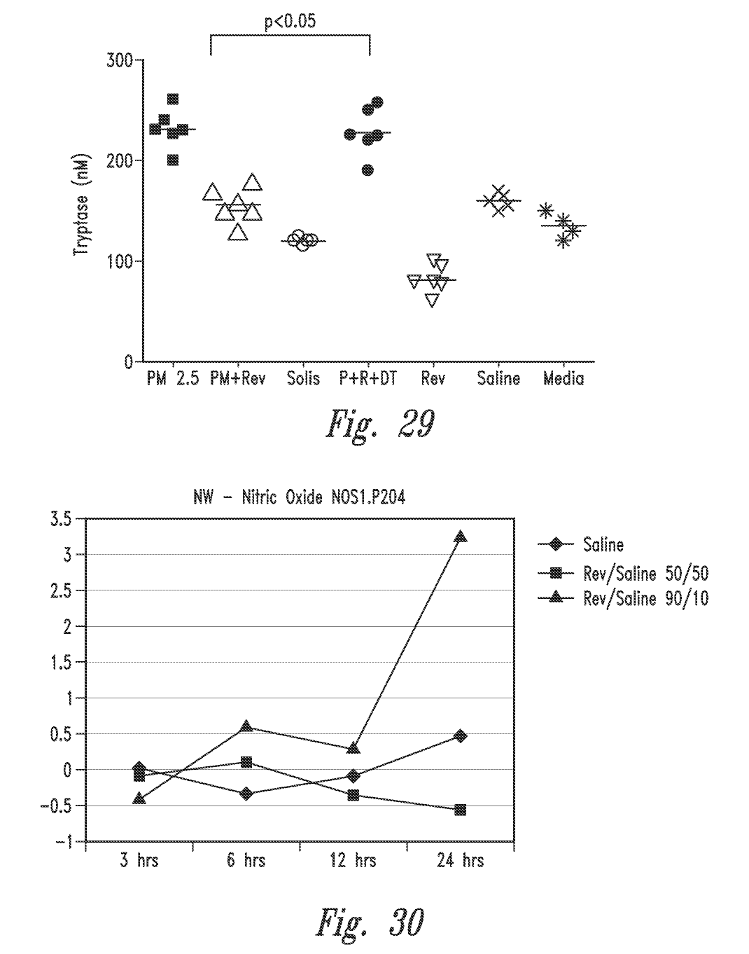

FIG. 29 shows AA PBMC data, obtained from an allergic asthma (AA) profile of peripheral blood mononuclear cells (PBMC) evaluating tryptase. The AA PBMC data was consistent with the above T-regulatory cell data, as cells stimulated with particulate matter (PM) showed high levels of tryptase, while cells treated with PM in the presence of the fluids of the instant disclosure (“PM+Rev”) resulted in significantly lower tryptase levels similar to those of the Saline and Media controls. Consistent with the data from T-regulatory cells, exposure to DT390 blocked the Rev-mediated effect on tryptase levels, resulting in an elevated level of tryptase in the cells as was seen for PM alone (minus Rev, no Rev, no Solis). Note that treatment with Rev alone resulted in lower tryptase levels relative to Saline and Media controls.

In summary, the data of FIG. 22, showing a decreased proliferation in the presence of PM and Rev relative to PM in control fluid (no Rev, no Solis), indicates that the inventive electrokinetically generated fluid Rev improved regulatory T-cell function as shown by relatively decreased proliferation in the assay. Moreover, the evidence of this Example and FIGS. 22-29, indicate that beta blockade, GPCR blockade and Ca channel blockade affects the activity of Revera on Treg function.

### EXAMPLE 8

### RNS-60 was Shown by Fluorescence-Activated Cell Sorting (FACS) Analysis to have a Pronounced Effect on Expression of Cell Surface Receptors: CD193 (CCR3); CD154 (CD4OL); CD11B; and CD3

Overview. Applicants used Fluorescence-Activated Cell Sorting (FACS) analysis to compare the levels of expression of cell surface receptors, CD193 (CCR3); CD154 (CD4OL); CD11B; and CD3, on white blood cells incubated with either the inventive electrokinetic fluid (RNS-60) or normal saline control fluid.

### Methods:

Ficoll-hypaque separated PBMC (apheresis—All Cells) preincubated approximately 1 hour in 30% solutions of RNS60 or Normal Saline (NS);

PBMC activated with 2 μg/ml of PHA-L for 24 or 40 hours;

Cells collected and washed into blocking/staining buffer, stained and fixed; and

Cells were analyzed by flow cytometry.

### Results:

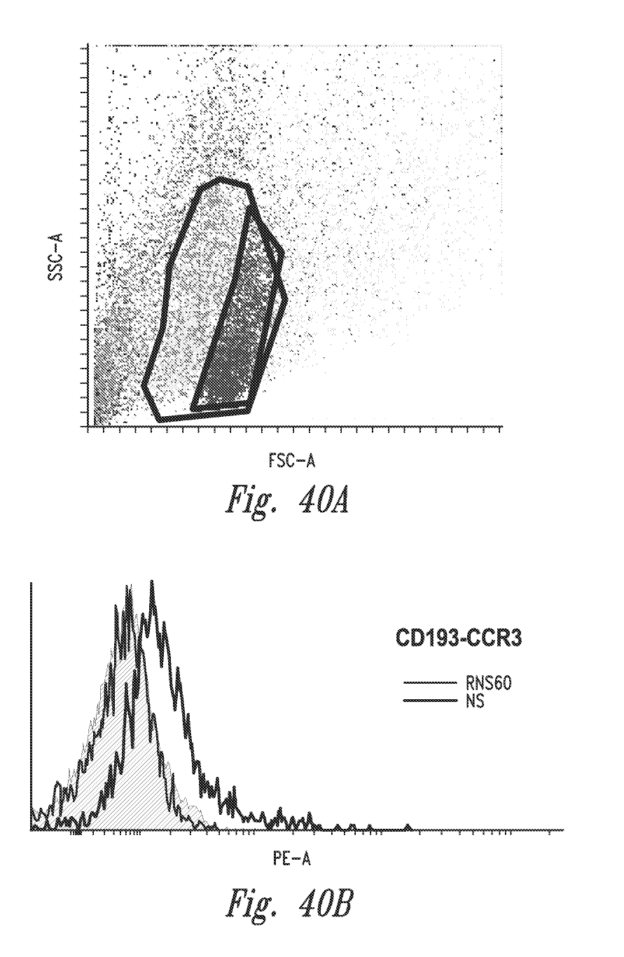

With respect to CD 193 (CCR3), as shown in FIG. 40B, the receptor is substantially down-regulated in the presence of RNS-60 when compared to the level of the receptor expression in the normal saline contol. This down regulation affects the phosphorylation of MAPK p38 (data not shown) which in turn down-regulates eotaxin (e.g., see Example 3 and FIG. 10) which in turn down regulates IL 5 (data not shown) and as well alters eosinophil counts (e.g., see Example 3), which is one of the factors that, that example, alters the bronchoconstrictive response.

As discussed above in Example 3 in the context of the ovalbumin challenge model and shown in FIG. 10, RNS-60 decreased the serum eotaxin levels in the OVA challenged groups when compared to the effect of normal saline. Therefore, according to particular aspects, RNS-60 has the potential to decrease both the ligand eotaxin and its receptor CCR3.

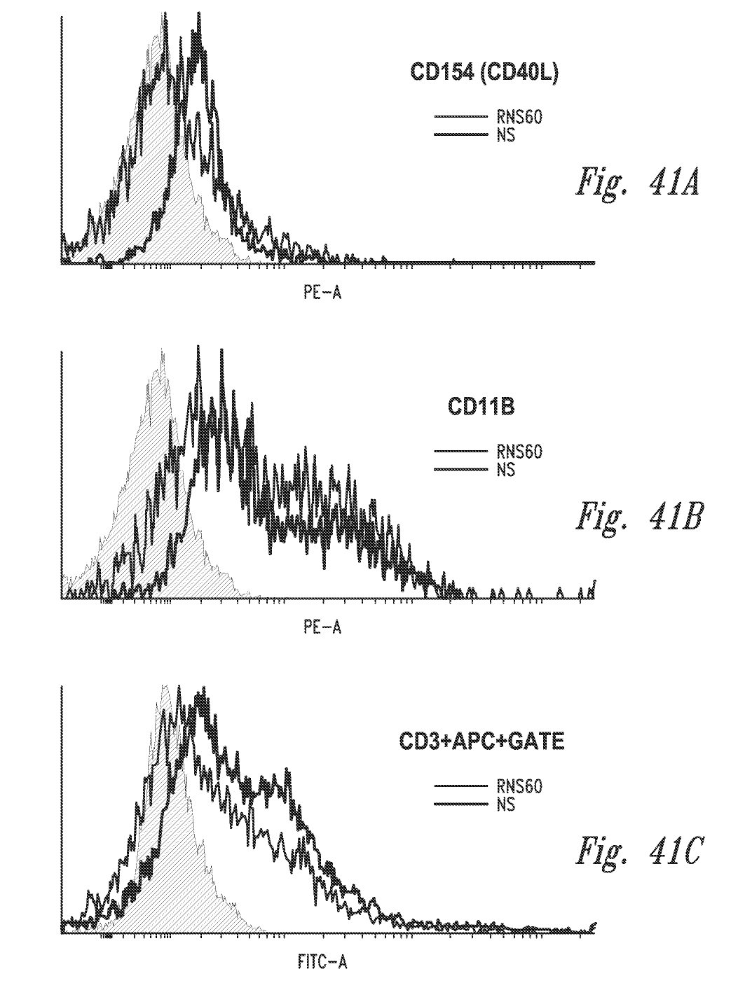

With respect to CD154 (CD4OL), as shown in FIG. 41A, the receptor is down-regulated in the presence of RNS-60 when compared to the level of the receptor expression in normal saline.

With respect to CD11B, as shown in FIG. 41B, the receptor is down-regulated in the presence of RNS-60 when compared to the level of the receptor expression in normal saline.

With respect to CD3, as shown in FIG. 41C, the receptor is down-regulated in the presence of RNS-60 when compared to the level of the receptor expression in normal saline.

### EXAMPLE 9

### RNS60, but Not Normal Saline (NS), Attenuated the Activation of NFκB in MBP-Primed T Cells

### Overview:

It is increasing clear that inhibition of insulin receptor signaling pathways is a central mechanism through which inflammatory and stress responses mediate insulin resistance (see, e.g., review by Wellen & Hotamisligil, The Journal of Clinical Investigation, 115:1111-1119, 2005).

Overlap of metabolic and immune pathways. Several serine/threonine kinases are activated by inflammatory or stressful stimuli and contribute to inhibition of insulin signaling, including JNK, inhibitor of NF-κB kinase (IKK), and PKC-θ (Zick, Y. 2003. Role of Ser/Thr kinases in the uncoupling of insulin signaling. Int. J. Obes. Relat. Metab. Disord. 27(Suppl. 3):S56-S60). Again, the activation of these kinases in obesity highlights the overlap of metabolic and immune pathways; these are the same kinases, particularly IKK and JNK, that are activated in the innate immune response by Toll-like receptor (TLR) signaling in response to LPS, peptidoglycan, double-stranded RNA, and other microbial products (Medzhitov, R. 2001. Toll-like receptors and innate immunity. Nat. Rev. Immunol. 1:135-145). Hence it is likely that components of TLR signaling pathways will also exhibit strong metabolic activities.

PKC and IKK are acitivated by cellular lipid metabolites. Two other inflammatory kinases that play a large role in counteracting insulin action, particularly in response to lipid metabolites, are IKK and PKC-θ. Lipid infusion has been demonstrated to lead to a rise in levels of intracellular fatty acid metabolites, such as diacylglycerol (DAG) and fatty acyl CoAs. This rise is correlated with activation of PKC-θ and increased Ser307 phosphorylation of IRS-1 (Yu, C., et al. 2002. Mechanism by which fatty acids inhibit insulin activation of insulin receptor substrate-1 (IRS-1)-associated phosphatidylinositol 3-kinase activity in muscle. J. Biol. Chem. 277:50230-50236). PKC-θ may impair insulin action by activation of another serine/threonine kinase, IKKβ, or JNK (Perseghin, G., Petersen, K., and Shulman, G. I. 2003. Cellular mechanism of insulin resistance: potential links with inflammation. Int. J. Obes. Relat. Metab. Disord. 27(Suppl. 3):S6-S11). Other PKC isoforms have also been reported to be activated by lipids and may also participate in inhibition of insulin signaling (Schmitz-Peiffer, C. 2002. Protein kinase C and lipid-induced insulin resistance in skeletal muscle. Ann. N Y. Acad. Sci. 967:146-157).

IKKβ can impact insulin signaling by activating NF-κB. IKKβ can impact on insulin signaling through at least 2 pathways. First, it can directly phosphorylate IRS-1 on serine residues (Yin, M. J., Yamamoto, Y., and Gaynor, R. B. 1998. The anti-inflammatory agents aspirin and salicylate inhibit the activity of IκB kinase-β. Nature. 396:77-80, Gao, Z., et al. 2002. Serine phosphorylation of insulin receptor substrate 1 by inhibitor kappa B kinase complex. J Biol. Chem. 277:48115-48121).

Second, it can phosphorylate inhibitor of NF-κB (IκB), thus activating NF-κB, a transcription factor that, among other targets, stimulates production of multiple inflammatory mediators, including TNF-α and IL-6 (Shoelson, S. E., Lee, J., and Yuan, M. 2003. Inflammation and the IKKβ/IκB/NF-κB axis in obesity- and diet-induced insulin resistance. Int. J. Obes. Relat. Metab. Disord. 27(Suppl. 3):S49-S52). Mice heterozygous for IKKβ are partially protected against insulin resistance due to lipid infusion, high-fat diet, or genetic obesity (Yuan, M., et al. 2001. Reversal of obesity- and diet induced insulin resistance with salicylates or targeted disruption of IKKβ Science. 293:1673-1677; Kim, J. K., et al. 2001. Prevention of fat-induced insulin resistance by salicylate. J. Clin. Invest. 108:437-446; doi:10.1172/JCI200111559).

Moreover, inhibition of IKKβ in human diabetics by high-dose aspirin treatment also improves insulin signaling, although at this dose, it is not clear whether other kinases are also affected (Hundal, R. S., et al. 2002. Mechanism by which high-dose aspirin improves glucose metabolism in type 2 diabetes. J. Clin. Invest. 109:1321-1326. doi:10.1172/JCI200214955). Recent studies have also begun to tease out the importance of IKK in individual tissues or cell types to the development of insulin resistance. Activation of IKK in liver and myeloid cells appears to contribute to obesity-induced insulin resistance, though this pathway may not be as important in muscle (Cai, D., et al. 2005. Local and systemic insulin resistance resulting from hepatic activation of IKKβ and NF-κB. Nat. Med. 11:183-190; Arkan, M. C., et al. 2005. IKKβ links inflammation to obesity-induced insulin resistance. Nat. Med. 11:191-198; and Rohl, M., et al. 2004. Conditional disruption of IκB kinase 2 fails to prevent obesity-induced insulin resistance. J. Clin. Invest. 113:474-481; doi:10.1172/JCI200418712).

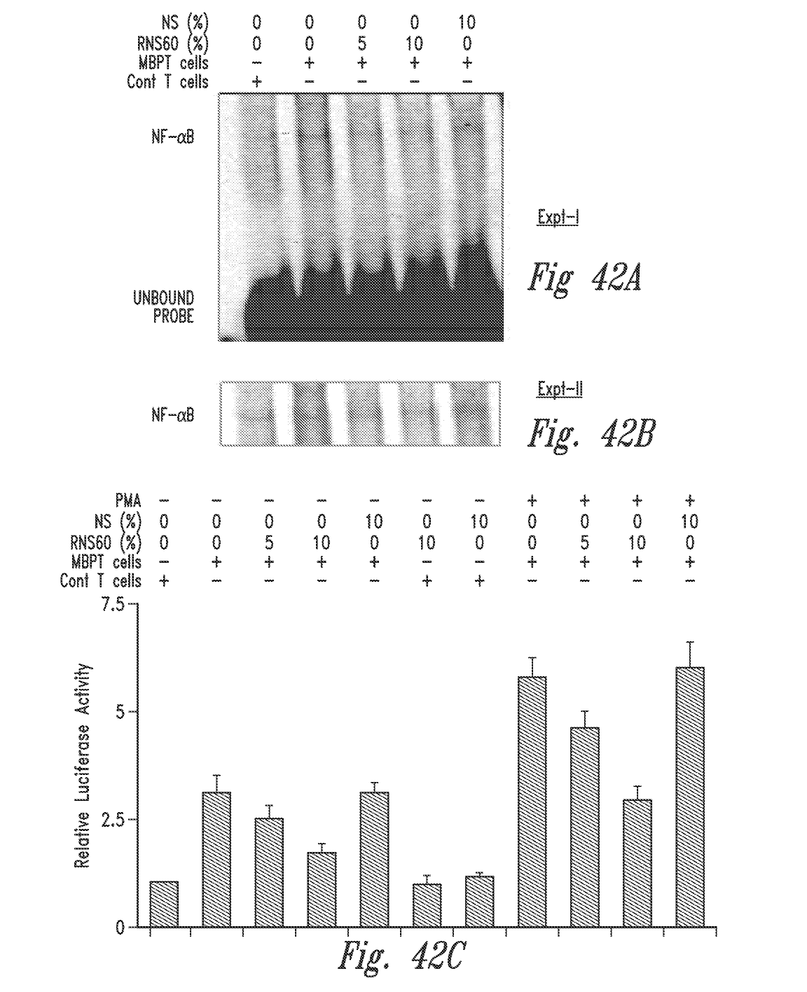

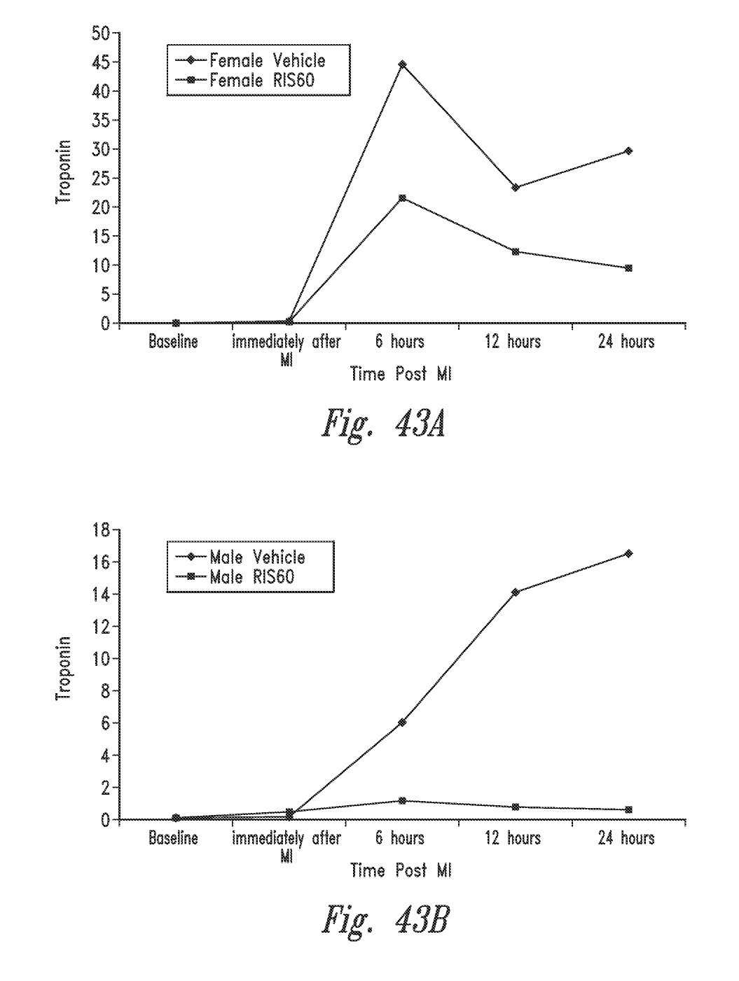

Methods. For the experiments shown in FIGS. 42A and 42B, T cells isolated from MBP-immunized mice were re-primed with MBP and after 24 h, cells received different concentrations of RNS60 and NS. After 2 h of treatment, DNA-binding activity of NF-κB was monitored in nuclear extracts by electrophoretic mobility shift assay (EMSA).

For experiments shown in FIG. 42C, T cells isolated from MBP-immunized mice were transfected with PBIIX-Luc, an NF-κB dependent reporter construct, followed by repriming with MBP. After 24 h of MBP priming, cells were treated with different concentrations of RNS60 and NS for 2 h followed by assay of luciferase activity in total cell extracts by a luciferase assay kit (Promega). In other cases, MBP-primed T cells were also stimulated with 30 nM PMA for 1 h. In these cases, PMA was added after 1 h of pretreatment with RNS60 and NS. Results are mean+SD of three different experiments.

Results. FIGS. 42A-C show that RNS60, but not normal saline (NS), attenuated the activation of NF-κB in MBP-primed T cells. Specifically, FIGS. 42A and 42B show that RNS60 (see middle three lanes of FIGS. 42A and 42B), but not NS (see right-most lane of FIGS. 42A and 42B), attenuated the activation of NF-κB in MBP-primed T cells in a dose-responsive manner.

Likewise, the bar graph of FIG. 42C shows that that RNS60 (see second, third and fourth bars of FIGS. 42A and 42B), but not NS (see fifth bar of FIGS. 42A and 42B), attenuated the activation of NF-κB in MBP-primed T cells, and hence also attenuated luciferase activity from the transfected NF-κB-dependent reporter construct (PBIIX-Luc) in total cell extracts, in a dose-responsive manner.

According to particular aspects, therefore, the disclosed electrokinetically-generated fluids have substantial utility for treating inflammation and inflammation-mediated conditions and diseases, including but not limited to, diabetes and related metabolic disorders, insulin resistance, neurodegenerative diseases (e.g., M.S., Parkinson's, Alzheimer's, etc), asthma, cystic fibrosis, vascular/coronary disease, retinal and/or macular degeneration, digestive disorders (e.g., inflammatory bowel disease, ulcerative colitis, Crohn's, etc.).

### EXAMPLE 10

### RNS60, but Not the Vehicle Control, Limited the Production of Troponin that Accumulated within 24 Hours Post Myocardial Infarction in Female and Male Pigs

### Overview:

Troponin levels in the blood are very sensitive and specific indicators of damage to the heart muscle. Although low levels normally are found in blood, a patient who suffered from a myocardial infarction would have an area of damaged heart muscle and thus would have elevated cardiac troponin levels in the blood. Thus elevated troponin blood levels would indicate that the heart muscle suffered from some sort of injury.

Methods. To confirm that RNS60 has a positive effect on survival and physiological parameters after myocardial infarction (MI), a study was conducted using the balloon-induced myocardial ischemia reperfusion (I/R) model in domestic pigs with a body weight of 45-50 kg. In this model, an angioplasty catheter is inserted surgically through the common carotid artery, and myocardial ischemia is caused by inflation of the balloon in the mid-left anterior descending (LAD) coronary artery. Ischemia was induced for a period of 40 minutes, after which the balloon was deflated resulting in reperfusion of the artery. Treatment was administered via a single intracoronary injection (3 mL) 5 minutes after reperfusion of the LAD, and subsequently by intravenous injection (100 mL/dose) every 4 h for a period of 7 days. Treatment groups comprised groups of males or females receiving either normal saline or RNS60 (4 groups total, TABLE 6).

TABLE 6
Study Groups

| Group Number | n | Sex | Test Material |
|---|---|---|---|
| 1F | 4 | Female | Normal Saline |
| 2M | 4 | Male | Normal Saline |
| 3F | 4 | Female | RNS60 |
| 4M | 4 | Male | RNS60 |

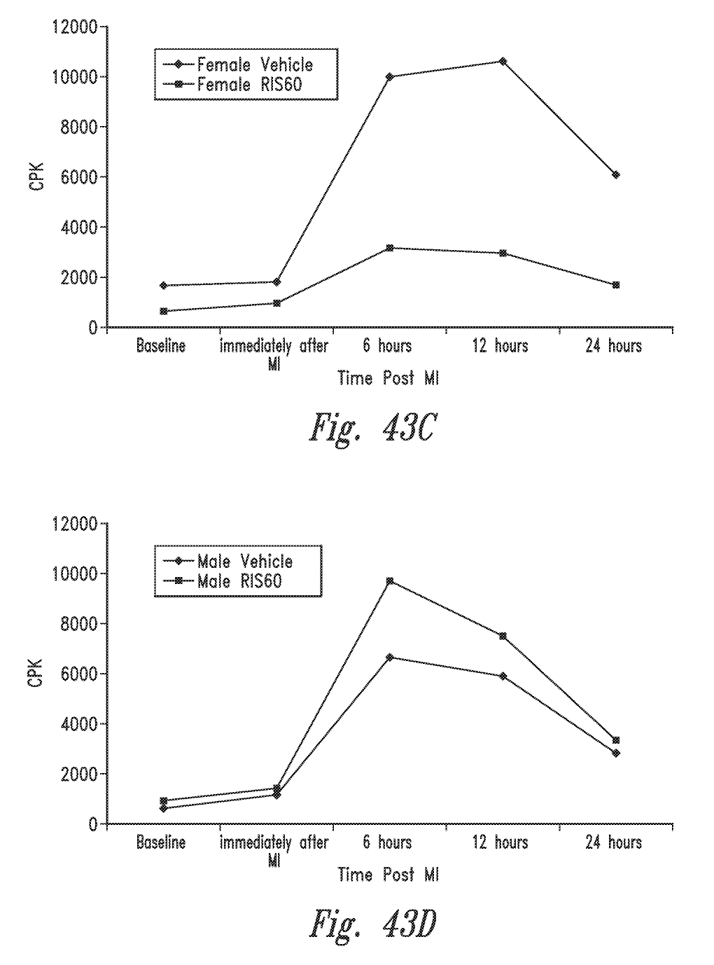

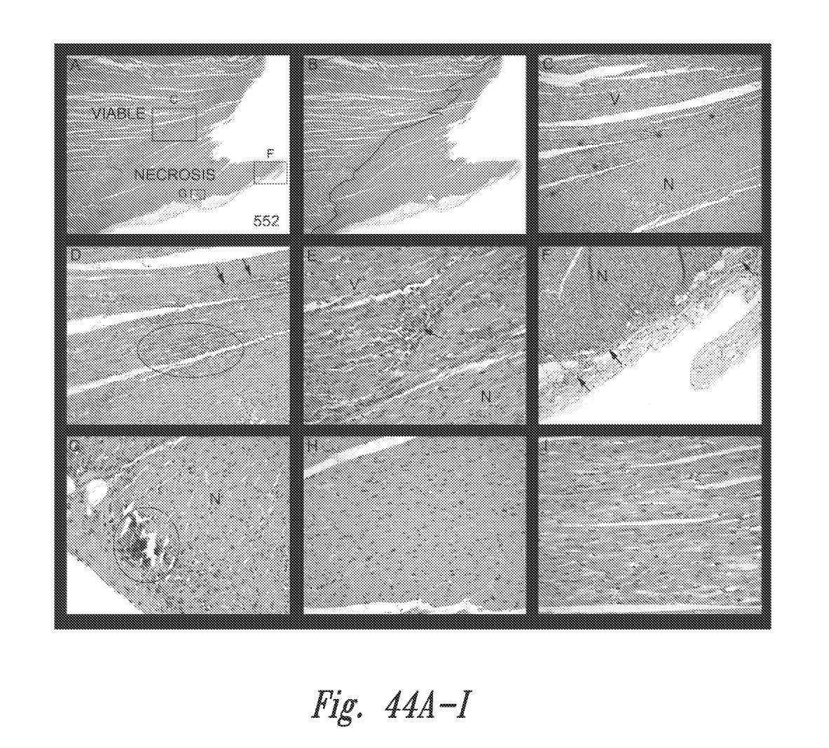

Specifically, for the experiments shown in FIGS. 43A and 43B, eight female (panel A) and eight male pigs (panel B) were treated with either RNS60 or vehicle alone and subjected to myocardial infarction. Prior to inducing myocardial infarction, each pig was injected with either RNS60 or vehicle alone. Myocardial infarction (MI) was induced in each animal by inflation of an angioplasty balloon (e.g., for 40 min) An angiogram was performed after inflation of the balloon and before deflation of the balloon in order to verify total occlusion of the coronary vessel and correct balloon positioning. After deflation of the balloon a subsequent angiogram was performed to verify restoration of blood flow in the previously occluded artery. Twenty minutes after balloon inflation, pigs were treated with 1 ml/min intracoronary infusion of either RNS60 or normal saline. A blood sample was taken from each animal prior to inducing MI to establish the baseline levels of troponin. Additional blood samples were taken at time points of immediately, six hours, twelve hours, and twenty-four hours after inducing the MI and assayed for troponin levels. Each point on the graph represents the average troponin blood level of the eight animals. Specifically, electrocardiogram (ECG), body weight, and heart rate were assessed and blood was collected on day −1, day 0 (between reperfusion and IC dosing as well as 6 and 12 hours later), day 1, and day 7 for biochemical cardiac marker analysis. At the end of the study, the hearts were perfused with formalin, removed, cut into 1 cm sections at the necrotic area (left ventricle) and evaluated macroscopically to grossly determine infarction size. For further histologic analysis, the anterior wall from each section was divided into three samples. Each sample was paraffin embedded and 5 μm sections were stained with H&E.

Results. The following results were obtained.

Survival. All female pigs survived the length of the study. One animal each from the normal saline control group and from the RNS60 treatment group were excluded from the results due to an apparent failure to induce ischemia based on ECG analysis. Among the males, two out of four normal saline-treated animals died on day 3 of the post-op housing period. In contrast, all RNS60-treated males survived the study.

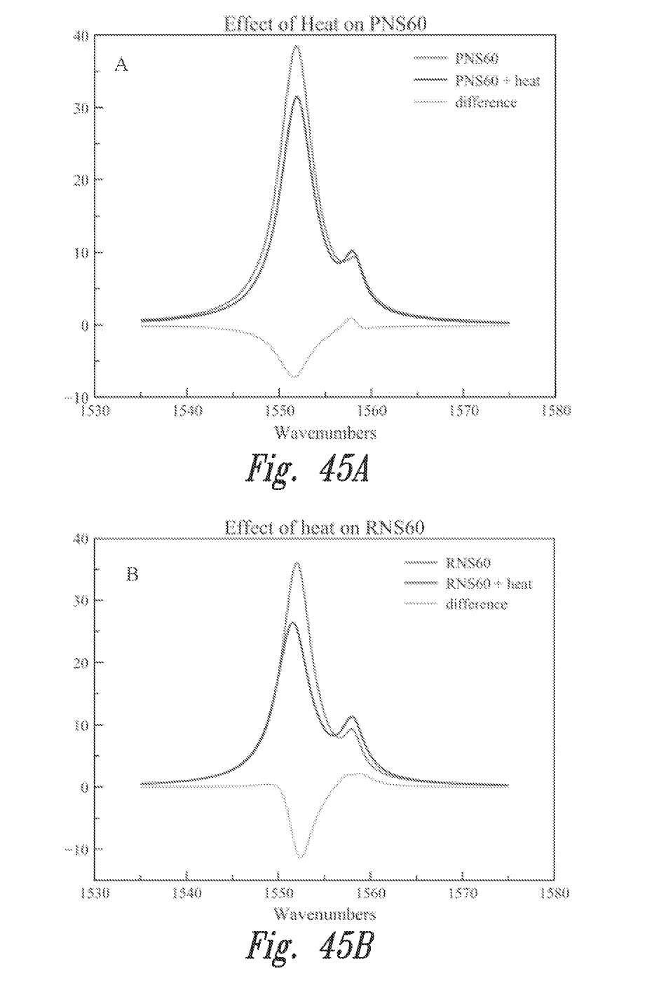

Infarct size. Based on gross histology, the infarct size in surviving animals overall affected about 2% to about 10% of the ventricle wall. Two of the three females in the control group displayed macroscopically visible infarcts. In contrast, two of the three females treated with RNS60 showed no signs of muscle damage. Only one male injected with normal saline and one male treated with RNS60 developed infarcts visible by gross histologic analysis. Microscopic changes related to infarction were found in the hearts of all control-treated and all RNS60-treated females, as well as in three out of the four control-treated males (TABLE 7). These changes included necrosis, fibrosis, mineralization, hemorrhage, and mononuclear infiltration (examples are shown in FIG. 44A-I). In contrast, none of the males treated with RNS60 showed any signs of myocardial damage on this level (TABLE 7).

TABLE 7
Summary of the microscopic histology results

| | | Infarct by |
|---|---|---|
| Treatment | Animal | microscopic hitology |
| Vehicle | 2910 | + |
| | 2919 | + |
| | 3078 | + |
| RNS60 | 2978 | + |
| | 2976 | + |
| | 2909 | + |
| Vehicle | 3038 | + |
| | 3031 | − |
| | 3080* | + |
| | 3033* | + |
| RNS60 | 3028 | − |
| | 3029 | − |
| | 3026 | − |
| | 3030 | − |

+ presence of infarcted tissue,
− = absence of infarcted tissue,
*died on day 3 after I/R

ECG changes. During the ischemic period, the ECG showed ST elevation in all animals enrolled into the study. In the immediate post-ischemic period (during IC drug administration), all female pigs of the control group showed T-wave abnormalities including T elevation, biphasic T waves, and flipped T waves. In contrast, two of the three RNS60-treated females were free of T-wave changes, suggesting an immediate treatment benefit. In the male groups, three out of four animals in both the control group and the RNS60 group showed T wave changes. At one week post-surgery, two of control-treated males had died and the remaining control animals continued to show changes in T waves. In contrast, two of the RNS60-treated males now showed normal ECG tracings.

Cardiac troponin. Treatment with RNS60 reduced the I/R-induced increase of circulating troponin levels in both male and female pigs (FIGS. 43A and B); the effect was more robust in males than in females. The two male animals that died on post-MI day 3 displayed the highest troponin levels among all males. FIGS. 43A and B show that RNS60, but not vehicle alone, significantly limited the production of troponin by approximately two- to twenty-fold over the vehicle alone treated animals. Specifically, FIGS. 43A (female) and B (male) shows that RNS60 (see line with squares of FIGS. 43A and B), reduced, by two- to three-fold for the female animals FIG. 43A and by five- to twenty-fold for males FIG. 43A, the blood levels of troponin in response to inducing the MI when compared to the vehicle alone (see line with diamonds of FIGS. 43A and B).

According to particular aspects, therefore, administration of RNS60 lowered mortality among male animals and exerted a normative trend on ECG changes following MI. The apparent benefits of RN60 treatment for myocardial tissue survival and preservation of physiological heart function were confirmed by reduced circulating levels of cardiac troponin and the absence of histologic signs of myocardial damage. The findings of this study display a consistent trend indicating anti-inflammatory and tissue-protective effects of RNS60 administration.

FIGS. 44A-I show, according to particular aspects, an example of the necrosis tissue found in a control-treated male animal (#3033). A. Low magnification showing necrotic tissue on the right and viable tissue on the left. The boxed areas are shown with higher magnification in C, F, and G, as indicated. B. The same photomicrograph shown in A with the interface between the viable and necrotic area marked by a black line. C. Medium magnification of the largest boxed area in A. Viable area (V) and necrotic area (N) are separated by tissue with basophilic appearance (*). D. Higher magnification of the central area in C. Most of the granular material is mineralized. A focus of mineralized cardiomyocytes is circled; intermixed are mononuclear inflammatory cells (arrows). E. High magnification of the interface between the viable (V) and necrotic (N) regions with multifocal mononuclear infiltration (arrow). F. Higher magnification of the epicardium overlying the necrotic area (boxed in A) shows mild multifocal mononuclear infiltration (arrows). G. Higher magnification of the tissue boxed in the bottom of A. Multifocal mineralization can be seen as granular basophilic material within necrotic cardiomyocytes. An area with heavy mineralization is circled. H. High magnification of necrotic myocardium. The nuclei of cardiomyocytes are pyknotic (small and darkly basophilic). The cytoplasm appears coagulated and has a distinctive eosinophilic hue. I. High magnification of viable myocardium. Note the difference in the appearance of nuclei and cytoplasm compared to H.

### EXAMPLE 11

### RNS60, but Not Vehicle Control, Attenuated the Level of Creatine Phosphokinase (CPK) that Accumlated within 24 Hours Post Myocardial Infarction in Female Pigs

### Overview:

Creatine phosphokinase (CPK) levels in the blood are very sensitvive and specific indicators of damage to the heart muscle. Although low levels normally are found in blood, if a patient who had suffered from a myocardial infarction would have an area of damaged heart muscle and thus would have elevated cardiac CPK levels in the blood.

Methods. The methods for this Example and as shown in FIGS. 43C and 43D were discussed in Example 10. A blood sample was taken from each animal prior to inducing MI to establish the baseline levels of CPK. Additional blood samples were taken at time points of immediately, six hours, twelve hours, and twenty-four hours after inducing the MI and assayed for CPK levels. Each point on the graph represents the average CPK blood level of the eight animals.

Results. FIGS. 43C (female pigs) show that RNS60 (see line with squares of FIGS. 43C), but not vehicle alone (see line with diamonds of FIGS. 43D), significantly limited the production of CPK by approximately three- to four-fold over the vehicle alone treated animals.

According to particular aspects, therefore, the results show that the animals treated with RNS60 produced lower levels of CPK, which indicates that the heart muscles had a reduced level of damage when treated with RNS60 compared to the vehicle alone.

### EXAMPLE 12

### The Disclosed Electrokinetically Altered Aqueous Fluids (e.g., RNS60, RIS60) Provide Adjuncts and/or Substitutes for Conventional Saline Solutions in the context of Surgery, Including Cardiovascular Surgery

Overview. According to additional aspects, the disclosed electrokinetically altered aqueous fluids (e.g., RNS60, RIS60) may augment or replace conventional solutions (e.g., saline-based solutions and fluids) used in the context of surgery, including but not limited to cardiovascular surgery to improve outcome and reduce deleterious effects attendant to various surgeries.

There are many cardiovascular surgical scenarios in which saline solutions are used and wherein it would be desirable to reduce or eliminate deleterious effects attendant to the surgical scenarious.

For example, in surgeries involving caridoplumonary bypass (CPB), where generalized inflammatory response causes deleterious effects, priming solution (bypass pump priming solution) comprising or consisting of the disclosed electrokinetically altered aqueous fluids (e.g., RNS60, RIS60) may be employed to reduce or eliminate deleterious effects attendant to CPB. According to further aspects, the disclosed electrokinetically altered aqueous fluids (e.g., RNS60, RIS60) may be employed to reduce deleterious neurocognitive effects of CPB.

Likewise, the disclosed electrokinetically altered aqueous fluids (e.g., RNS60, RIS60) may be employed in the context of vein preservation solution (typically papaverine and saline) to enahance outcome.

Additionally, the disclosed electrokinetically altered aqueous fluids (e.g., RNS60, RIS60) may be employed as cadioplegia (e.g., glutamate and/or aspartate-containing cardiplegia, that may also contain potassium) to flush down coronaries after grafts are completed.

According to further aspects, therefore, the disclosed electrokinetically altered aqueous fluids (e.g., RNS60, RIS60) may be employed anywhere in surgery (e.g., cardiovascular surgery) to augment or replace solutions (e.g., saline-based solutions and fluids) conventionally used in the surgery (e.g., cardiovascular surgery) to improve outcome and reduce deleterious effects attendant to various surgeries.

Representative cardiovascular related surgeries include aortic coarctation repair, Blalock-Taussig shunt creation, closure of patent ductus arteriosus, treating complications of ischemic heart disease (for example, coronary artery bypass grafting), correcting congenital heart disease, treating valvular heart disease caused by various causes including endocarditis, treat coronary artery disease, treating valvular heart disease, treating tumors of the heart, thoracic aortic aneurysm repair, valve surgery, aortic surgery, arrhythmia (atrial fibrillation) surgery, heart failure and transplant surgery, minimally invasive heart surgery, video-assisted and robotic-assisted cardiac surgery, heart valve procedures, transmyocardial laser revascularization, thoracic aortic aorta procedures, angioplasty (e.g., percutaneous transluminal angioplasty (PTA)), angiography, carotid endarterectomy, aortic aneurysm repair, balloon valvuloplasty, cerebral aneurysm repair, cardioversion, aortic valve replacement, carotid endarterectomy, arteriovenous fistula, cardiac catheterization, aortic valve replacement, pericardiocentesis, mitral valve repair, mitral valve replacement, endotracheal intubation, peripheral vascular bypass surgery, coronary stenting, enhanced external counterpulsation, endovascular stent surgery, portal vein bypass, heart-lung transplantation, implantable cardioverterdefibrillator, peripheral endarterectomy, venous thrombosis prevention, and myocardial resection.

Particular aspects provide methods for performing a surgery, comprising performing a surgery on a subject in need thereof, wherein a reagent fluid is used in at least one aspect of the surgery, and wherein the reagent fluid comprises a surgically effective amount of an electrokinetically altered aqueous fluid comprising an ionic aqueous solution of charge-stabilized oxygen-containing nanostructures substantially having an average diameter of less than about 100 nanometers.

In particular aspects, the surgery comprises at least one selected from the group consisting of: surgery related to cardiac arrhythmia; surgery related to vascular disease; surgery related to myocardial infarction; surgery related to congestive heart failure; surgery related to myocarditis; surgery related to atherosclerosis, and restenosis; surgery comprising use of caridoplumonary bypass (CPB); surgery comprising use of vessel (e.g., vein, artery) preservation solution; and surgery comprising use of cadioplegia.

### EXAMPLE 13

### Patch Clamp Analysis Conducted on Calu-3 Cells Perfused with Inventive Electrokinetically Generated Fluids (RNS-60 and Solas) Revealed that (i) Exposure to RNS-60 and Solas Resulted in Increases in Whole Cell Conductance, (ii) that Exposure of Cells to the RNS-60 Produced an Increase in a Non-Linear Conductance, Evident at 15 min Incubation Times, and (iii) that Exposure of Cells to the RNS-60 Produced an Effect of RNS-60 Saline on Calcium Permeable Channels

Overview. This Example is based on Example 17 of Applicants' published U.S. patent application Ser. No. 12/435,356, incorporated herein by reference for its teachings regarding modulation membrane conductance by electrokinetic fluids. In this Example, patch clamp studies were performed to further confirm the utilities, as described herein, of the inventive electrokinetically generated saline fluids (RNS-60 and Solas), including the utility to modulate whole-cell currents. Two sets of experiments were conducted.

The summary of the data of the first set of experiments showed that the whole cell conductance (current-to-voltage relationship) obtained with Solas saline is highly linear for both incubation times (15 min, 2 hours), and for all voltage protocols. It was, however, evident that longer incubation (2 hours) with Solas increased the whole cell conductance. Exposure of cells to the RNS-60 produced an increase in a non-linear conductance, which was only evident at 15 min incubation time. The effect of the RNS-60 on this non-linear current disappeared, and instead was linear at the two-hour incubation time. The contribution of the non-linear whole cell conductance, as previously observed, was voltage sensitive, although present at all voltage protocols.

The summary of data of the second set of experiments indicates that there is an effect of the RNS-60 saline on a non-linear current, which was made evident in high calcium in the external solution. The contribution of the non-linear whole cell conductance, although voltage sensitive, was present in both voltage protocols, and indicates an effect of RNS-60 saline on calcium permeable channels.

### First Set of Experiments (Increase of Conductance; and Activation of a Non-Linear Voltage Regulated Conductance

### Methods for First Set of Experiments:

See cellular patch clamp working Example 23 from WO 2009/055729 for general patch clamp methods. In the following first set of experiments, patch clamp studies were performed to further confirm the utility of the inventive electrokinetically generated saline fluids (RNS-60 and Solas) to modulate whole-cell currents, using Calu-3 cells under basal conditions, with protocols stepping from either zero mV holding potential, −120 mV, or −60 mV.

The whole-cell conductance in each case was obtained from the current-to-voltage relationships obtained from cells incubated for either 15 min or two hours. In this study, groups were obtained at a given time, for either Solas or RNS-60 saline solutions.

### Results:

The results showed a series of patch clamping experiments that assessed the effects of the electrokinetically generated fluid (e.g., RNS-60 and Solas) on epithelial cell membrane polarity and ion channel activity at two time-points (15 min and 2 hours) and at different voltage protocols (A, stepping from zero mV; B, stepping from −60 mV; and C, stepping from −120 mV). The results indicated that the RNS-60 has a larger effect on whole-cell conductance than Solas. In the experiment similar results were seen in the three voltage protocols and at both the 15 minute and two-hour incubation time points.

Further results showed the subtraction of the Solas current data from the RNS-60 current data at three voltage protocols (“Delta currents”) (A, stepping from zero mV; B, stepping from −60 mV; and C, stepping from −120 mV) and the two time-points (15 mins and 2 hours). These data indicated that at the 15 minute time-point with RNS-60, there is a non-linear voltage-dependent component that is absent at the 2 hour time point.

As in previous experiments, data with “Normal” saline gave a very consistent and time-independent conductance used as a reference. The present results were obtained by matching groups with either Solas or RNS-60 saline, and indicated that exposure of Calu-3 cells to the RNS-60 saline under basal conditions (without cAMP, or any other stimulation), produces time-dependent effect(s), consistent with the activation of a voltage-regulated conductance at shorter incubation times. This phenomenon was not as apparent at the two-hour incubation point. As described elsewhere herein, the linear component is more evident when the conductance is increased by stimulation with the cAMP “cocktail”. Nonetheless, the two-hour incubation time showed higher linear conductance for both the RNS-60 and the Solas saline, and in this case, the RNS-60 saline doubled the whole cell conductance as compared to Solas alone. This evidence indicates that at least two contributions to the whole cell conductance are affected by the RNS-60 saline, namely the activation of a non-linear voltage regulated conductance, and a linear conductance, which is more evident at longer incubation times.

### Second Set of Experiments (Effect on Calcium Permeable Channels)

### Methods for Second Set of Experiments:

In the following second set of experiments, yet additional patch clamp studies were performed to further confirm the utility of the inventive electrokinetically generated saline fluids (RNS-60 and Solas) to modulate whole-cell currents, using Calu-3 cells under basal conditions, with protocols stepping from either zero mV or −120 mV holding potentials.

The whole-cell conductance in each case was obtained from the current-to-voltage relationships obtained from cells incubated for 15 min with either saline. To determine whether there is a contribution of calcium permeable channels to the whole cell conductance, and whether this part of the whole cell conductance is affected by incubation with RNS-60 saline, cells were patched in normal saline after the incubation period (entails a high NaCl external solution, while the internal solution contains high KCl). The external saline was then replaced with a solution where NaCl was replaced by CsCl to determine whether there is a change in conductance by replacing the main external cation. Under these conditions, the same cell was then exposed to increasing concentrations of calcium, such that a calcium entry step is made more evident.

### Results:

In general, the results of a series of patch clamping experiments that assessed the effects of the electrokinetically generated fluid (e.g., Solas and RNS-60) on epithelial cell membrane polarity and ion channel activity using different external salt solutions and at different voltage protocols (stepping from zero mV or stepping from −120 mV) showed that whole cell conductance was increased. In these experiments one time-point of 15 minutes was used. For Solas the results indicated that: 1) using CsCl instead of NaCl as the external solution, increased whole cell conductance with a linear behavior when compared to the control; and 2) CaCl₂ at both 20 mM CaCl₂ and 40 mM CaCl₂ increased whole cell conductance in a non-linear manner. For RNS-60, the results indicate that: 1) using CsCl instead of NaCl as the external solution had little effect on whole cell conductance when compared to the control; and 2) CaCl₂ at 40 mM increased whole cell conductance in a non-linear manner.

Additional results showed the subtraction of the CsCl data from the 20 mM CaCl₂ and 40 mM CaCl₂ data at two voltage protocols (stepping from zero mV and stepping from −120 mV) for Solas and RNS-60. The results indicate that both Solas and RNS-60 solutions activated a calcium-induced non-linear whole cell conductance. The effect was greater with RNS-60 (indicating a dosage responsiveness), and with RNS-60 was only increased at higher calcium concentrations. Moreover, the non-linear calcium dependent conductance at higher calcium concentration was also increased by the voltage protocol.

The data of this second set of experiments further indicates an effect of RNS-60 saline and Solas saline for whole cell conductance data obtained in Calu-3 cells. The data indicated that 15-min incubation with either saline produces a distinct effect on the whole cell conductance, which is most evident with RNS-60, and when external calcium is increased, and further indicates that the RNS-60 saline increases a calcium-dependent non-linear component of the whole cell conductance.

The accumulated evidence suggests activation by Revalesio saline of ion channels, which make different contributions to the basal cell conductance.

Taken together with Applicants' other data (e.g., the data of Applicants other working Examples) particular aspects of the present invention provide compositions and methods for modulating intracellular signal transduction, including modulation of at least one of membrane structure, membrane potential or membrane conductivity, membrane proteins or receptors, ion channels, lipid components, or intracellular components with are exchangeable by the cell (e.g., signaling pathways, such as calcium dependant cellular signaling systems, comprising use of the inventive electrokinetically generated solutions to impart electrochemical and/or conformational changes in membranous structures (e.g., membrane and/or membrane proteins, receptors or other membrane components) including but not limited to GPCRs and/or g-proteins. According to additional aspects, these effects modulate gene expression, and may persist, dependant, for example, on the half lives of the individual messaging components, etc.

### EXAMPLE 14

### Atomic Force Microscopy (AFM) Measurements of the Inventive Electrokinetic Fluid (RNS-60) Indicated the Presence and/or Formation of Hydrophobic Surface Nanobubbles that were Substantially Smaller that those Present in Control ‘Pressure Pot’ (PNS-60) Fluid

Overview. This Example is based on Example 18 of Applicants' published U.S. patent application Ser. No. 12/435,356, incorporated herein by reference for its teachings regarding nanobubbles. Applicants used Atomic Force Microscopy (AFM) measurements to characterize hydrophobic nanobubbles in the inventive electrokinetic fluid (RNS-60).

### Materials and Methods:

AFM studies. AFM studies were preformed at an art-recognized Nanotech User Facility (NTUF). For AFM studies, a very small and sensitive needle is dipped into a droplet of water placed onto a hydrophobic surface. The needle then scans over the water/surface interface at rates such as 1 mm² in ˜15 minutes. The needle records any imperfections in the surface geometry, and is sensitive enough to record the presence of small bubbles.

The Silicon substrate upon which the water droplets were placed was prepared using Trichloro(1H,1H,2H,2H-perfluorooctyl)silane), and the resulting hydrophobic surface causes water to bead up with contact angles of approximately 95 degrees. This coating is used in many AFM studies, in part, because it is particularly durable.

Solution Preparation. Two test solutions were studied: RNS-60 and PNS-60. RNS-60 is an inventive electrokinetic fluid comprising 60 ppm oxygen, whereas PNS-60 is a non-electrokinetic control fluid comprising 60 ppm oxygen prepared by conventional exposure to a pressurized oxygen head (i.e., pressure pot oxygenated fluid). Each test solution was initially buffered by addition of a small amount of neutral phosphate buffer (pH 7) solution, and approximately 60-70 uL of each buffered test solution (approximately 22° C.) was placed onto a previously prepared silica plate.

### Results:

Under AFM, the RNS-60 droplet displayed a distribution of about 20 hydrophobid nanobubbles in a 1 mm² area, having dimensions of ˜20 nm wide and ˜1.5 nm tall or smaller. By contrast, under AFM, the PNS-60 droplet displayed approx 5 hydrophobic nanobubbles in a 1 mm² area, having dimensions of ˜60 nm wide and ˜5 nm tall. The PNS-60 droplet, therefore, had much fewer and much larger hydrophobic nanobubbles compared to the RNS60 droplet.

According to particular aspects, therefore, there is a substantial difference in the size and distribution of hydrophobic surface nanobubbles between the RNS-60 and PNS-60 test solutions, where the nanobubbles are either initially present in, and/or formed within the test fluids during AFM measurement.

As discussed elsewhere herein, according to particular aspects of the present invention, the inventive electrokinetically altered fluids comprise an ionic aqueous solution of charge-stabilized oxygen-containing nanostructures substantially having an average diameter of less than about 100 nanometers and stably configured in the ionic aqueous fluid in an amount sufficient to provide, upon contact of a living cell by the fluid, modulation of at least one of cellular membrane potential and cellular membrane conductivity.

Applicants point out, however, that the hydrophobic bubbles (forming on a hydrophobic surface), such as those observed in AFM experiments are likely fundamentally different from inventive biologically-active charge-stabilized nanostructure disclosed herein. According to particular aspects therefore, while the AFM experiments in this working Example support, based on the size and distribution hydrophobic bubble formation, that the inventive electrokinetic fluids (e.g., RNS-60) are fundamentally distinct from non-electrokinetic control fluids, the hydrophobic bubbles are likely distinct from and/or derived from the inventive charge-stablilized oxygen-containing nanostrutures described in detail elsewhere herein. In any event, relative to the inventive electrokinetic fluids, control pressure pot oxygenated fluids do not comprise charge-stabilized oxygen-containing nanostructures capable of modulation of at least one of cellular membrane potential and cellular membrane conductivity.

### EXAMPLE 15

### Raman Spectroscopy was Used to Distinguish Electrokinetic Fluids (e.g., RNS-60) from Control Pressure Pot' (PNS-60) Fluid

FIGS. 45A and B show, according to particular aspects, the effect of increased temperature (heart) on RNS60 (45B) relative to control PNS60 (45A), as measured by Raman backscatter, showing respective difference curves, and two oxygen peaks.

According to particular aspects, Raman spectroscopy has substantial utility for characterizing electrokinetically-altered fluids (e.g., RNS60). According to further aspects, such Raman spectroscopy data/measurements is correlated with biological activity of the electrokinetically-altered fluids (e.g., RNS60).

### EXAMPLE 16

### Fluorescence Polarization Anisotropy was Used to Distinguish Electrokinetic Fluids (e.g., RNS-60) from Control Fluids

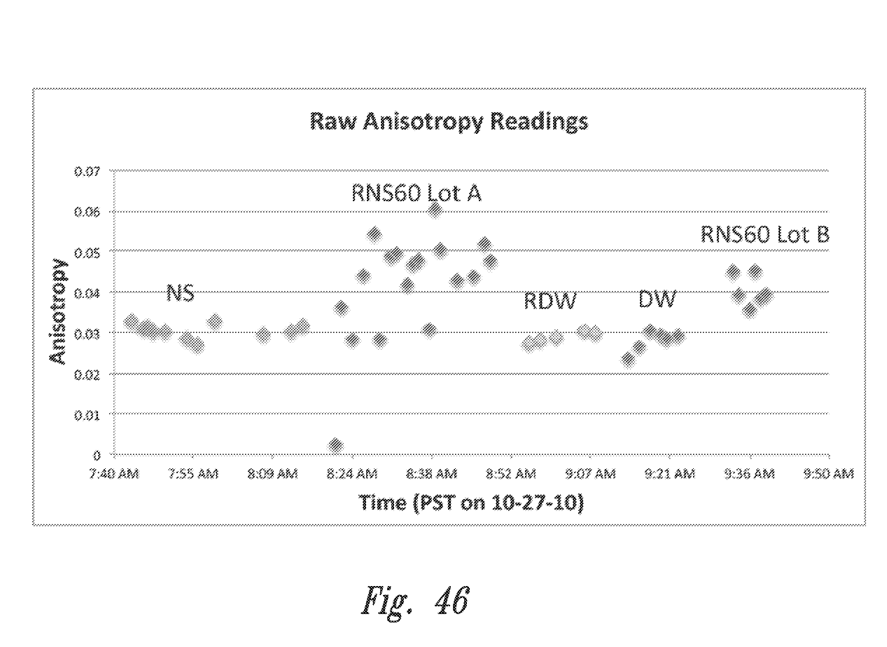

FIG. 46 shows, according to particular aspects, small but significant differences in fluorescence polarization anisotropy data between and among “RNS60” (“Lot A” and “Lot B”), “NS” (normal saline control), “RDW” (electrokinetically processed deionized water) and “DW” (deionized water).

According to particular aspects, fluorescence polarization anisotropy has substantial utility for characterizing electrokinetically-altered fluids (e.g., RNS60). According to further aspects, such fluorescence polarization anisotropy data/measurements is correlated with biological activity of the electrokinetically-altered fluids (e.g., RNS60).

### EXAMPLE 17

### Electrokinetic Fluids (e.g., RNS60) were Shown to Interact with Biological Membranes as Evidenced by Modulation of Ion Channel Activity, or Electrical Spiking

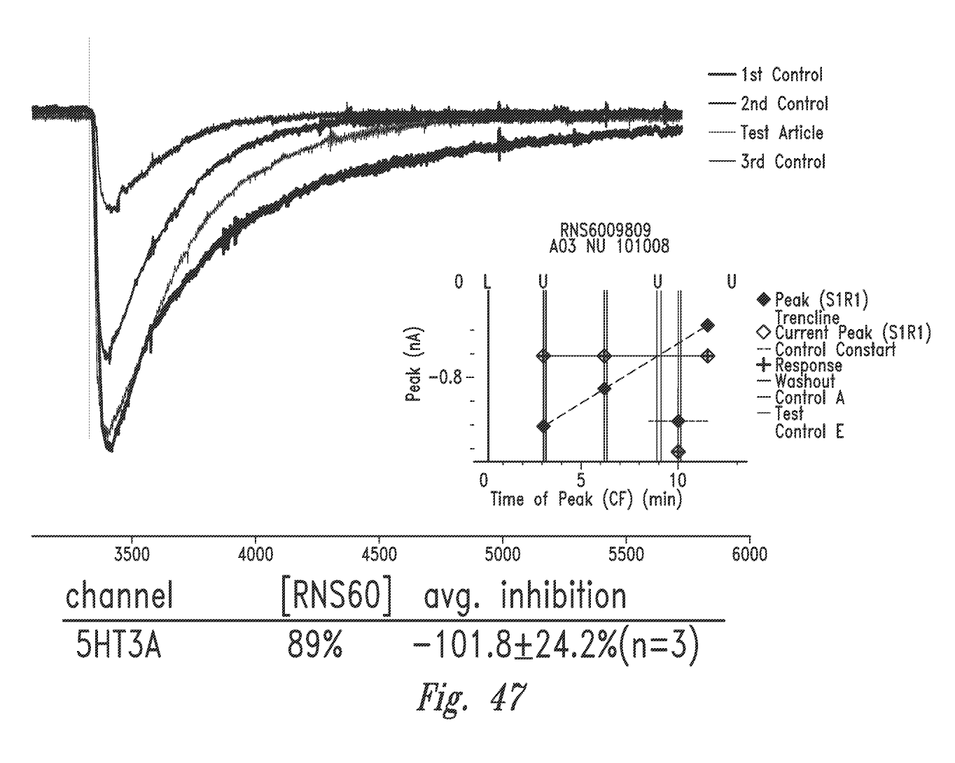

FIG. 47 shows, according to particular aspects, that extracellularly perfused RNS60 (89%) potentiates serotonin-evoked 5HT3A (ion channel) activity (avg. inhibition of −101.8±24.2% (n=3), relative to control).

According to particular aspects, measurement of effects on serotonin-evoked 5HT3A (ion channel) activity has substantial utility for characterizing electrokinetically-altered fluids (e.g., RNS60). According to further aspects, such measurement of effects on serotonin-evoked 5HT3A (ion channel) activity is correlated with biological activity of the electrokinetically-altered fluids (e.g., RNS60).

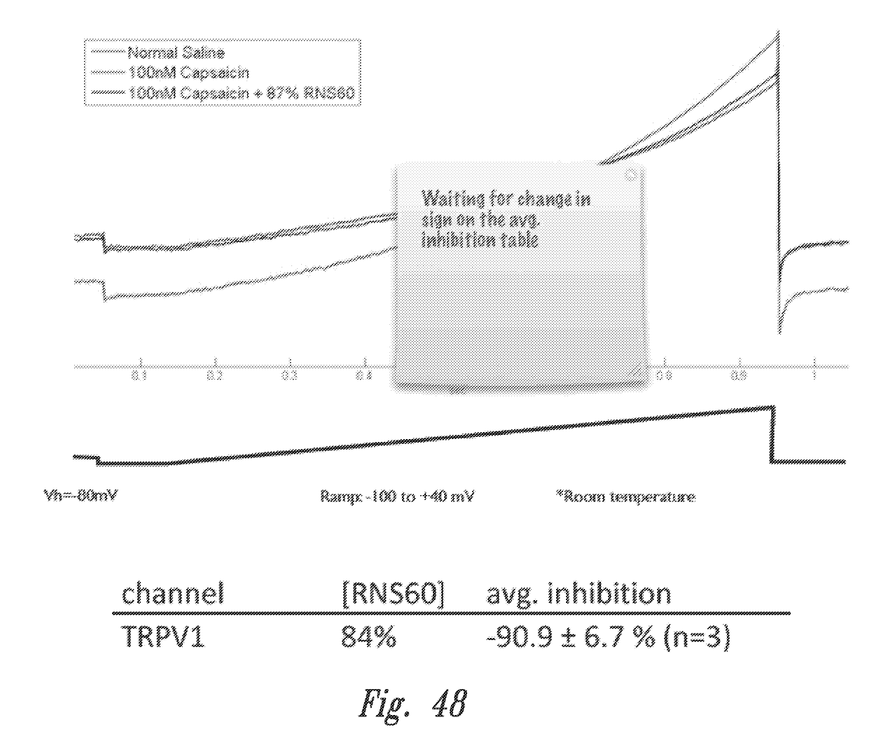

FIG. 48 shows, according to particular aspects, that RNS60 (84%) inhibits capsaicin evoked TRPV1 (ion channel) currents (avg. inhibition of −90.9±6.7% (n=3). Comparison is between Normal Saline, 100 nm Capsaicin, and 100 nm Capsaicin +87% RNS60. Data shown is that for recombinant CHO cells overexpressing TRPV1.

According to particular aspects, measurement of effects on capsaicin evoked TRPV1 (ion channel) currents has substantial utility for characterizing electrokinetically-altered fluids (e.g., RNS60). According to further aspects, such measurement of capsaicin evoked TRPV1 (ion channel) currents is correlated with biological activity of the electrokinetically-altered fluids (e.g., RNS60).

According to particular aspects, modulation of ion channel activity reflects the RNS60 interaction with biological membranes. According to additional aspects, ion channel measurements (e.g., potentiation, inhibition, alteration of gating kinetics, voltage sensitivity, etc.), including e.g., modulation of agonist-evoked activity, have substantial utility in facile and high-throughput methods for measuring the biological activity of electronkinetically-altered fluids (e.g., RNS60).

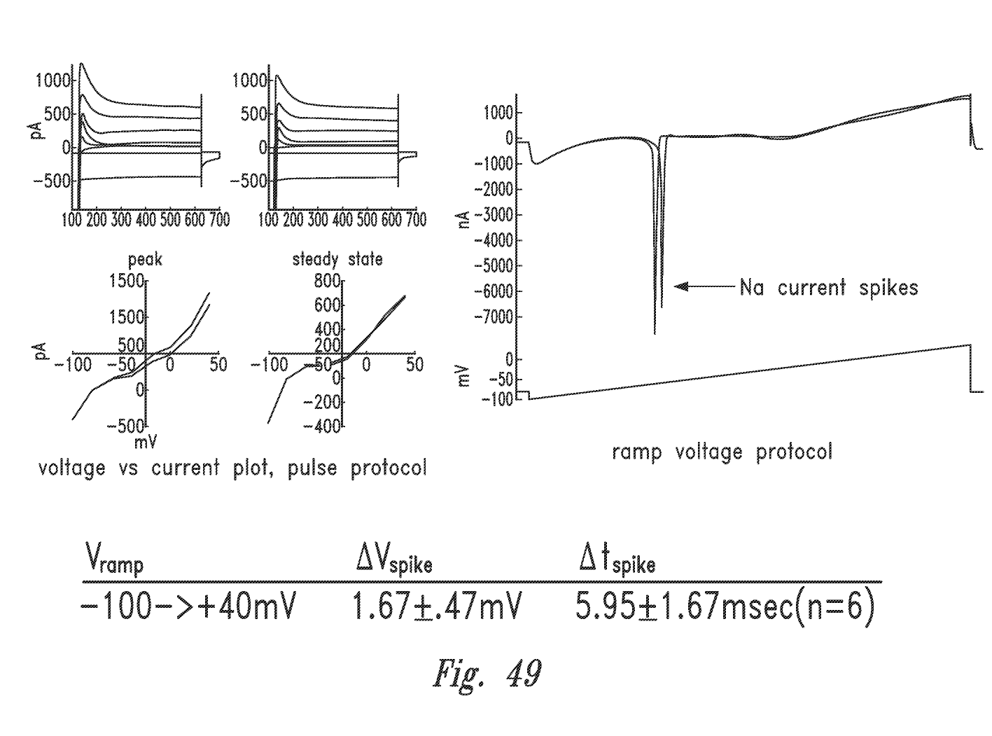

FIG. 49 shows, according to particular aspects, that perfusion with RNS60 alters electrical spiking (Na+ current spikes) in cardiomyocytes; Vᵣₐₘₚ, −100 mV→+40 mV; ΔVₛₚᵢₖₑ, 1.67±0.47 mV; Δtₛₚᵢₖₑ, 5.95 1.67 msec (n=6).

According to particular aspects, measurement of electrical spiking (e.g., (Na+ current spikes)) in cardiomyocytes has substantial utility for characterizing electrokinetically-altered fluids (e.g., RNS60). According to further aspects, such measurement of electrical spiking in cardiomyocytes is correlated with biological activity of the electrokinetically-altered fluids (e.g., RNS60). Without being bound by mechanism, delay of spiking may be due to interaction with Nav1.5.

Incorporation by Reference. All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

It should be understood that the drawings and detailed description herein are to be regarded in an illustrative rather than a restrictive manner, and are not intended to limit the invention to the particular forms and examples disclosed. On the contrary, the invention includes any further modifications, changes, rearrangements, substitutions, alternatives, design choices, and embodiments apparent to those of ordinary skill in the art, without departing from the spirit and scope of this invention, as defined by the following claims. Thus, it is intended that the following claims be interpreted to embrace all such further modifications, changes, rearrangements, substitutions, alternatives, design choices, and embodiments.

The foregoing described embodiments depict different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Accordingly, the invention is not limited except as by the appended claims.

1. A method for treating a cardiovascular disease or condition, comprising administering to a subject, or portion thereof, in need thereof a therapeutically effective amount of an electrokinetically altered aqueous fluid comprising an ionic aqueous solution of charge-stabilized oxygen-containing nanostructures predominantly having an average diameter of less than about 100 nanometers and stably configured in the ionic aqueous fluid in an amount sufficient to provide for treating a cardiovascular disease or condition or at least one symptom thereof.

2. The method of claim 1, wherein the charge-stabilized oxygen-containing nanostructures are stably configured in the ionic aqueous fluid in an amount sufficient to provide, upon contact of a living cell by the fluid, modulation of at least one of cellular membrane potential and cellular membrane conductivity.

3. The electrokinetic fluid of claim 1, wherein the charge-stabilized oxygen-containing nanostructures are the major charge-stabilized gas-containing nanostructure species in the fluid.

4. The electrokinetic fluid of claim 1, wherein the percentage of dissolved oxygen molecules present in the fluid as the charge-stabilized oxygen-containing nanostructures is a percentage selected from the group consisting of greater than: 0.01%, 0.1%, 1%, 5%; 10%; 15%; 20%; 25%; 30%; 35%; 40%; 45%; 50%; 55%; 60%; 65%; 70%; 75%; 80%; 85%; 90%; and 95%.

5. The electrokinetic fluid of claim 1, wherein the total dissolved oxygen is substantially present in the charge-stabilized oxygen-containing nanostructures.

6. The electrokinetic fluid of claim 1, wherein the charge-stabilized oxygen-containing nanostructures predominantly have an average diameter of less than a size selected from the group consisting of: 90 nm; 80 nm; 70 nm; 60 nm; 50 nm; 40 nm; 30 nm; 20 nm; 10 nm; and less than 5 nm.

7. The electrokinetic fluid of claim 1, wherein the ionic aqueous solution comprises a saline solution.

8. The electrokinetic fluid of claim 1, wherein the fluid is superoxygenated.

9. The electrokinetic fluid of claim 1, wherein the fluid comprises a form of solvated electrons.

10. The method of claim 1, wherein alteration of the electrokinetically altered aqueous fluid comprises exposure of the fluid to hydrodynamically-induced, localized electrokinetic effects.

11. The method of claim 10, wherein, exposure to the localized electrokinetic effects comprises exposure to at least one of voltage pulses and current pulses.

12. The method of claim 10, wherein the exposure of the fluid to hydrodynamically-induced, localized electrokinetic effects, comprises exposure of the fluid to electrokinetic effect-inducing structural features of a device used to generate the fluid.

13. The method of claim 1, wherein the cardiovascular disease or condition comprises at least one condition or disease selected from the group consisting of cardiac arrhythmia, vascular disease, myocardial infarction, congestive heart failure, myocarditis, atherosclerosis, and restenosis.

14. The method of claim 13, wherein the cardiovascular condition or disease comprises at least one of myocardial infarction, congestive heart failure, myocarditis, and atherosclerosis.

15. The method of claim 14, wherein the cardiovascular condition or disease comprises at least one of myocardial infarction and atherosclerosis.

16. The method of claim 1, wherein the at least one symptom of cardiovascular disease is related to at least one condition selected from the group consisting of: cardiac arrhythmia, vascular disease, myocardial infarction, congestive heart failure, myocarditis, atherosclerosis, and restenosis.

17. The method of claim 1, wherein the electrokinetically altered aqueous fluid modulates localized or cellular levels of nitric oxide.

18. The method of claim 1 wherein the electrokinetically altered aqueous fluid promotes a localized decrease at the site of administration of at least one cytokine selected from the group consisting of: IL-1beta, IL-8, TNF-alpha, and TNF-beta.

19. The method of claim 1, further comprising a synergistic or non-synergistic inhibition or reduction in inflammation by simultaneously or adjunctively treating the subject with another anti-inflammatory agent.

20. The method of claim 19, wherein said other anti-inflammatory agent comprises a steroid or glucocorticoid steroid.

21. The method of claim 20, wherein the glucocorticoid steroid comprises Budesonide or an active derivative thereof

22. The method of claim 1, further comprising combination therapy, wherein at least one additional therapeutic agent is administered to the patient.

23. The method of claim 22, wherein the at least one additional therapeutic agent is selected from the group consisting of: quinidine, procainamide, disopyramide, lidocaine, phenytoin, mexiletine, flecainide, propafenone, moricizine, propranolol, esmolol, timolol, metoprolol, atenolol, bisoprolo, amiodarone, sotalol, ibutilide, dofetilide, dronedarone, E-4031, verapamil, diltiazem, adenosine, digoxin, magnesium sulfate, warfarin, heparins, anti-platelet drugs (e.g., aspirin and clopidogrel), beta blockers (e.g., metoprolol and carvedilol), angiotensin-converting enzyme (ACE) inhibitors (e.g., captopril, zofenopril, enalapril, ramipril, quinapril, perindopril, lisinopril, benazepril, fosinopril, casokinins and lactokinins), statins (e.g., atorvastatin, cerivastatin, fluvastatin, lovastatin, pitavastatin, mevastatin, pravastatin, rosuvastatin, and simvastatin), aldosterone antagonist agents (e.g., eplerenone and spironolactone), digitalis, diuretics, digoxin, inotropes (e.g., Milrinone), vasodilators and omega-3 fatty acids and combinations thereof

24. The method of claim 22, wherein the at least one additional therapeutic agent is a TSLP and/or TSLPR antagonist.

25. The method of claim 24, wherein the TSLP and/or TSLPR antagonist is selected from the group consisting of neutralizing antibodies specific for TSLP and the TSLP receptor, soluble TSLP receptor molecules, and TSLP receptor fusion proteins, including TSLPR-immunoglobulin Fc molecules or polypeptides that encode components of more than one receptor chain.

26. The method of claim 2, wherein modulation of at least one of cellular membrane potential and cellular membrane conductivity comprises modulating at least one of cellular membrane structure or function comprising modulation of a conformation, ligand binding activity, or a catalytic activity of a membrane associated protein.

27. The method of claim 26, wherein the membrane associated protein comprises at least one selected from the group consisting of receptors, transmembrane receptors, ion channel proteins, intracellular attachment proteins, cellular adhesion proteins, integrins, etc.

28. The method of claim 27, wherein the transmembrane receptor comprises a G-Protein Coupled Receptor (GPCR).

29. The method of claim 28, wherein the G-Protein Coupled Receptor (GPCR) interacts with a G protein a subunit.

30. The method of claim 29, wherein the G protein a subunit comprises at least one selected from the group consisting of Gαₛ, Gαᵢ, Gα_{q}, and Gα₁₂.

31. The method of claim 30, wherein the at least one G protein a subunit is Ga_{q}.

32. The method of claim 2, wherein modulating cellular membrane conductivity, comprises modulating whole-cell conductance.

33. The method of claim 32, wherein modulating whole-cell conductance, comprises modulating at least one voltage-dependent contribution of the whole-cell conductance.

34. The method of claim 2, wherein modulation of at least one of cellular membrane potential and cellular membrane conductivity comprises modulating intracellular signal transduction comprising modulation of a calcium dependant cellular messaging pathway or system.

35. The method of claim 2, wherein modulation of at least one of cellular membrane potential and cellular membrane conductivity comprises modulating intracellular signal transduction comprising modulation of phospholipase C activity.

36. The method of claim 2, wherein modulation of at least one of cellular membrane potential and cellular membrane conductivity comprises modulating intracellular signal transduction comprising modulation of adenylate cyclase (AC) activity.

37. The method of claim 2, wherein modulation of at least one of cellular membrane potential and cellular membrane conductivity comprises modulating intracellular signal transduction comprising modulation of intracellular signal transduction associated with at least one condition or symptom selected from the group consisting of: chronic inflammation in the cardiovascular system, and acute inflammation in the cardiovascular system.

38. The method of claim 1, comprising administration to a cell network or layer, and further comprising modulation of an intercellular junction therein.

39. The method of claim 38, wherein the intracellular junction comprises at least one selected from the group consisting of tight junctions, gap junctions, zona adherins and desmasomes.

40. The method of claim 38, wherein the cell network or layers comprises at least one selected from the group consisting of endothelial cell and endothelial-astrocyte tight junctions in CNS vessels, blood-cerebrospinal fluid tight junctions or barrier, pulmonary epithelium-type junctions, bronchial epithelium-type junctions, and intestinal epithelium-type junctions.

41. The method of claim 1, wherein the electrokinetically altered aqueous fluid is oxygenated, and wherein the oxygen in the fluid is present in an amount of at least 8 ppm, at least 15, ppm, at least 25 ppm, at least 30 ppm, at least 40 ppm, at least 50 ppm, or at least 60 ppm oxygen at atmospheric pressure.

42. The method of claim 1, wherein the amount of oxygen present in charge-stabilized oxygen-containing nanostructures of the electrokinetically-altered fluid is at least 8 ppm, at least 15, ppm, at least 20 ppm, at least 25 ppm, at least 30 ppm, at least 40 ppm, at least 50 ppm, or at least 60 ppm oxygen at atmospheric pressure.

43. The method of claim 1, wherein the electrokinetically altered aqueous fluid comprises at least one of a form of solvated electrons, and electrokinetically modified or charged oxygen species.

44. The method of claim 43, wherein the form of solvated electrons or electrokinetically modified or charged oxygen species are present in an amount of at least 0.01 ppm, at least 0.1 ppm, at least 0.5 ppm, at least 1 ppm, at least 3 ppm, at least 5 ppm, at least 7 ppm, at least 10 ppm, at least 15 ppm, or at least 20 ppm.

45. The method of claim 43, wherein the electrokinetically altered oxygenated aqueous fluid comprises solvated electrons stabilized, at least in part, by molecular oxygen.

46. The method of claim 1, wherein the ability to alter cellular membrane structure or function sufficient to provide for modulation of intracellular signal transduction persists for at least two, at least three, at least four, at least five, at least 6, at least 12 months, or longer periods, in a closed gas-tight container.

47. The method of claim 26, wherein the membrane associated protein comprises CCR3.

48. The method of claim 1, wherein treating comprises modulation of intracellular NF-κB expression and/or activity, preferably decreasing NF-κB expression and/or activity.

49. The method of claim 1, wherein the subject is a mammal or human.

50. A method of performing a surgery, comprising:
performing a surgery on a subject in need thereof, wherein a reagent fluid is used in at least one aspect of the surgery, and wherein the reagent fluid comprises a surgically effective amount of an electrokinetically altered aqueous fluid comprising an ionic aqueous solution of charge-stabilized oxygen-containing nanostructures substantially having an average diameter of less than about 100 nanometers.

51. The method of claim 50, wherein the surgery is a cardiovascular surgery.

52. The method of claim 51, wherein the surgery comprises at least one selected from the group consisting of: surgery related to cardiac arrhythmia; surgery related to vascular disease; surgery related to myocardial infarction; surgery related to congestive heart failure; surgery related to myocarditis; surgery related to atherosclerosis, and restenosis; surgery comprising use of caridoplumonary bypass (CPB); surgery comprising use of vessel (e.g., vein, artery) preservation solution; and surgery comprising use of cadioplegia.

53. A method for facile high-throughput measurement of biological activity of electronkinetically-altered fluids (e.g., RNS60), comprising:
contacting a cell with an electronkinetically-altered fluid as defined herein;
performing, using a suitable assay, an ion-channel measurement; and
determining, based on the ion-channel measurement relative to that of cells contacted with control fluid, a biological activity level or value of the electronkinetically-altered fluid.

54. The method of claim 53, wherein the ion-channel measurement is at least one selected from the group consisting of potentiation, inhibition, alteration of gating kinetics, voltage sensitivity, and modulation of agonist-evoked activity.

55. The method of claim 53, wherein the ion channel is at least one of 5HT3A and TRPV1.

56. The method of claim 55, comprising measurement of at least one of serotonin-evoked 5HT3A and capsaicin evoked TRPV1.

57. A method for facile high-throughput measurement of biological activity of electronkinetically-altered fluids (e.g., RNS60), comprising:
performing at least one of Raman spectroscopy and fluorescence polarization anisotropy measurement on an ionic aqueous solution of charge-stabilized oxygen-containing nanostructures predominantly having an average diameter of less than about 100 nanometers; and
correlating the at least one Raman spectroscopy and fluorescence polarization anisotropy measurement with an amount of biological activity of the electrokinetically-altered fluid, wherein a method for facile high-throughput measurement of biological activity of the electronkinetically-altered fluid is afforded.

58. The method of claim 57, comprising measurement of at least one of Raman backscatter and fluorescence polarization anisotropy.
